(19) United States
(12) Patent Application Publication
(10) Pub. No.: US 2024/0423702 A1
(43) Pub. Date: Dec. 26, 2024

(54) SYSTEMS AND METHODS FOR RF-ASSISTED LIPOSUCTION

(71) Applicant: CYNOSURE, LLC, Westford, MA (US)

(72) Inventors: James Boll, Auburndale, MA (US); Samuel Bruce, Malden, MA (US); Samuel McGinn, Billerica, MA (US); Paul Mannion, Eliot, ME (US); Oscar Ayala, Houston, TX (US); Bo Chen, Los Altos, CA (US); Mirko Mirkov, Chelmsford, MA (US); Noah Hamlen, Somerville, MA (US); David Sonnenshein, Boxford, MA (US); Daniel Masse, Windham, NH (US)

(73) Assignee: CYNOSURE, LLC, Westford, MA (US)

(21) Appl. No.: 18/755,616

(22) Filed: Jun. 26, 2024

Related U.S. Application Data
(60) Provisional application No. 63/523,304, filed on Jun. 26, 2023.

(51) Int. Cl. A61B 18/14; A61B 18/00

(52) U.S. Cl. CPC A61B 18/148; A61B 2018/00464; A61B 2018/00625; A61B 2018/00642; A61B 2018/00702; A61B 2018/00732; A61B 2018/00875

(57) ABSTRACT

In part, in one aspect, the disclosure relates to a cosmetic tissue treatment probe. The probe may include a cannula having a length. The cannula includes a conductive material, an inner surface, an outer surface, a cannula wall disposed between the inner surface and the outer surface, a proximal end, a distal end, a tip, and one or more apertures defined by the cannula wall. The cannula wall has a cannula wall thickness and a cannula length. In some embodiments, at least a portion of the tip is defined by the distal end. The conductive material is configured for electrical communication with an RF generator, the conductive material configured to generate a heat effect in response to a RF signal received from the RF generator, wherein the heat effect is generated relative to a medium or tissue in fluid communication with the one or more apertures.

### PRIORITY

This application claims priority to and benefit of U.S. Provisional Patent Application No. 63/523,304, filed on Jun. 26, 2023, the entirety of which is incorporated by reference herein.

### FIELD

The present disclosure relates generally to systems and methods for treating a patient's skin (e.g., dermis and hypodermis), adipose layers and other target tissue with radiofrequency (RF) energy.

### BACKGROUND

For individuals who seek to surgically modify the contours of their body, liposuction is a common procedure, normally practiced by plastic surgeons. In 2019, plastic surgeons performed 1.4 million liposuction procedures worldwide, second only to breast augmentations. The procedure was first done using suction and a hollow cannula in 1975 and underwent a paradigm shift in 1987 with the introduction of large volumes of very dilute aesthesia (tumescence) prior to the suction step to aid in vasoconstriction to reduce blood loss and post operation bruising.

The procedure is physically demanding for the surgeon, requiring multiple full arm and shoulder reciprocating motions of the cannula fanning out from one of several incision points. Depending on the patient's tissue structure and history of prior surgeries in the area of treatment, this area can be fibrous and resist the liposuction probe from advancing in the tissue. Procedures can take hours and practitioner fatigue is common.

Over the years, advances have taken place to assist the physician in faster, less physically demanding liposuction. Power assisted liposuction (PAL) is commonly used to address this. PAL uses a motorized reciprocating (˜40 strokes per second) tube inside the cannula that helps break up the fat near the suction port. While this improves the rate of tissue removal versus standard liposuction, the present disclosure improves upon and addresses some of the challenges regarding the state-of-the-art liposuction procedures and devices.

### SUMMARY

Ultrasound assisted liposuction (UAL) may be used to disrupt fat. The UAL procedure is followed by a separate procedure, specifically, by standard suction-assisted liposuction (SAL) procedure, which adds to the procedure time. In some embodiments, UAL is advantageously practiced with simultaneous suction as disclosed herein. Laser-assisted liposuction (LAL) may be implemented in some embodiments similar to UAL using only laser to disrupt fat and adding a second step of SAL.

The instant disclosure relates in part to the use of radio-frequency assisted liposuction (RFAL) that may be configured to assist in the speed or case of fat removal in a single system. The RFAL disclosure improves on multi system procedures like, for example, the UAL and SAL combination procedure and the LAL and SAL combination procedure, because the disclosed RFAL procedure can be accomplished with a single system.

In part, the disclosure relates to a RFAL system that includes a housing; one or more probe connectors; a first pump disposed in the housing; a radio-frequency (RF) generator disposed in the housing; the RF generator configured to output treatment energy signals at one or more modes or two or more modes; a power supply disposed in the housing, the power supply in electrical communication with the first pump and the RF generator; and a control system disposed in the housing, the control system in electrical communication with the RF generator and/or the first pump, wherein the control system adjusts the operation of the RF generator in response to one or more user inputs, wherein the control system is configured to operate the RF generator in one or more modes.

In some embodiments, the one or more modes is a pulsed RF energy mode otherwise referred to as a pulsed RF power mode. In some embodiments, the one or more modes is a continuous RF power mode. And in still other embodiments, the systems utilizes continuous RF power mode for some aspects of treatment and pulsed RF energy mode for other aspects of treatment. In some embodiments, the first pump is a suction pump (e.g., a vacuum pump) configured to create suction in a probe connected to the one or more probe connectors, wherein an opening of the probe is in fluid communication with the suction pump (e.g., the vacuum pump). In some embodiments, a first pump is an infiltration pump (e.g., a peristaltic pump) configured to deliver one or more fluids to a probe, wherein an opening of the probe is in fluid communication with the peristaltic pump. Optionally, a second pump may be a second peristaltic pump configured to deliver one or more fluids to a probe when an opening of the probe is in fluid communication with the peristaltic pump. In various embodiments, the system may further include a user input device in electrical communication with the control system, wherein the user input device receives one or more user inputs and delivers one or more control signals in response thereto to the control system. In some embodiments, the user input device comprises one or more foot pedal switches. In some embodiments, the device comprises a first probe. In some embodiments, the first probe may be selected from the group of a liposuction probe, a RF tissue treatment handpiece, a monopolar RF delivery probe, a bipolar RF delivery probe, a cannula-based probe, a suction assisted liposuction probe, a RF-assisted liposuction probe and an RF-based suction-assisted bubble generation liposuction probe. In some embodiments, the first probe is a cannula-based probe comprising a substantially cylindrical housing, the housing defining three or more ports.

In some embodiments, the three or more ports in the probe include a first port, a second port, and a third port, wherein the first port is an opening at a first end of the substantially cylindrical housing, wherein the second port is an opening at a second end of the substantially cylindrical housing, wherein the third port is an opening defined by a sidewall of the substantially cylindrical housing. In some embodiments, the first probe is a radio frequency assisted liposuction (RFAL). In various embodiments, the system may further include a first probe, wherein the first probe is an RFAL probe, the RFAL probe comprising one or more sensors.

In some embodiments, at least one of the ports is a thermally insulated tissue surface (skin) port. In various embodiments, the system may further include a ring structure, the ring structure disposed along the length of the cylindrical housing. In some embodiments, the RF generator is configured to output one or more modes and/or frequencies configured to cause a portion of a probe or a probe tip to generate bubbles in response thereto. In various embodiments, one or more of the sensors is a sound or acoustic wave detection sensor. In some embodiments, the system may further include a display, the display in electrical communication with the control system, the display configured to output a graphical user interface, wherein the graphical user interface shows one or more of the following: infiltrate rate, liposuction RF level, liposuction RF pulsing characteristics, treatment temperature, heating RF temperature, heating RF level, heating RF pulse characteristics, vacuum level, wherein one or more of the following change during a treatment session.

Many embodiments of the disclosure are designed to streamline the systems and methods and avoid reliance on multiple different probes and associated technologies and support systems. Floor space and wall service is at a premium in medical practices that endeavor to offer several procedures requiring different medical device platforms. Various embodiments of the disclosure are configured to improve workflow, usability, and use of floor space and wall services (e.g., to lessen demand for electrical wall service) through the design of a given system, the associated disposables, and the treatment methods and as otherwise disclosed herein.

### Cosmetic Probes and Related Features and Exemplary Embodiments

In part, in one aspect, the disclosure relates to a cosmetic tissue treatment probe. The probe may include a cannula having a length. The cannula includes a conductive material, an inner surface, an outer surface, a cannula wall disposed between the inner surface and the outer surface, a proximal end, a distal end, a tip, and one or more apertures defined by the cannula wall. The cannula wall has a cannula wall thickness and a cannula length. In some embodiments, at least a portion of the tip is defined by the distal end, the proximal end attachable to a handle. The conductive material is configured for electrical communication with an RF generator, the conductive material configured to generate a heat effect in response to a RF signal received from the RF generator, wherein the heat effect is generated relative to a medium or tissue in fluid communication with the one or more apertures.

In various embodiments, the RF signal has one or more probe parameters, the one or more probe parameters selected to adjust or achieve the heat effect or a heat effect parameter. In some embodiments, the one or more probe parameters of the RF signal that are selected include at least two of pulsed RF, continuous wave RF, peak power of the RF signal, average power of RF signal, a repetition rate of RF signal, on-time of the RF signal and a RF frequency. In some embodiments, the heat effect parameter is selected from one or more of the following: volume of heating, duration of heating, gradient of heating, amount of heat localization, heating efficiency for localized change of state or steam formation, heating efficiency for localized tissue effects, RF efficiency for localized change of state or plasma formation, and heating efficiency for localized change of state or plasma formation.

In various embodiments, a heating efficiency for localized tissue effects includes one or more of more of localized tissue coagulation, localized tissue ablation or vaporization, localized mechanical disruption, localized thermo-mechanical tissue disruption, localized pressure disruption, or localized shockwave tissue disruption. In some embodiments, the one or more probe parameters are peak power, average power, an energy per pulse and a repetition rate, wherein the pulsed RF signal has a peak power having a range of about 200 W to about 3 kW, an average power having a range of about 5 W to about 100 W, an energy per pulse range of about 1 J to about 5 J per pulse and a repetition rate range of about 5 Hz to about 100 Hz. In various embodiments, the one or more probe parameters are peak power, average power, an energy per pulse, wherein the pulsed RF energy signal has a peak power having a range of about 200 W to about 3 kW, an average power having a range of 0.1 W to 10 W, an energy per pulse range of 1 J to 5 J per pulse and a repetition rate of about 0.5 Hz to about 10 Hz. In some embodiments, the one or more probe parameters are peak power, average power, an energy per pulse, wherein the pulsed RF signal has a peak power having a range of 200 W to 3 KW, an average power having a range of about 1 W to about 60 W, an energy per pulse range of 0.5 J to 3 J per pulse and a repetition rate of about 1 Hz to about 60 Hz.

In some embodiments, the one or more apertures are holes or channels that span the cannula wall thickness. In some embodiments, the one or more apertures are slots defined by the inner surface and the exterior surface of the cannula, wherein each slot defines a channel disposed through the cannula wall. In various embodiments, the one or more apertures includes a first aperture having a first perimeter and a second aperture having a second perimeter, wherein a shape of the first perimeter is substantially the same as a shape of the second perimeter, wherein the cannula has a longitudinal axis, wherein a first point within the first perimeter and a second point within the second perimeter are colinear along a line segment substantially perpendicular to the longitudinal axis.

In some embodiments, the probe may further include a second set of two apertures disposed through the cannula wall thickness of two cannula walls that face one another along the length of the cannula and are placed at a location ninety degrees off set from the other set of two apertures such that about the perimeter of the cannula there are four apertures each having centers that are approximately equidistant from the center of the next aperture about the perimeter of the cannula.

In some embodiments, the probe may further include an insulative layer disposed on at least a portion of a surface or edge of the cannula. In various embodiments, the cannula further includes a port for communication with a first pump or a second pump, wherein the first pump is a suction pump, the suction pump configured suction pump for removal of unwanted tissue through the cannula. In some embodiments, the second pump is an infiltration pump for delivery of tumescent fluid through the cannula.

In some embodiments, the application of the RF signal is selected based on one or more parameters of the RF signal to promote heat generation regions in an aqueous medium surrounding the cannula. In various embodiments, the probe may further include a control system, the control system in electrical communication with one or more electrical contacts of the probe, wherein the control system is programmed to terminate delivery of an RF signal during treatment when a measured reactance value is received or generated by the control system that is indicative of plasma formation. In some embodiments, application of the pulsed RF energy signal initiates, promotes or causes formation of one or more heat bubbles in an aqueous medium surrounding the cannula and in fluid communication with the one or more apertures.

In various embodiments, the one or more apertures includes two apertures each disposed through the cannula wall thickness and adjacent to one another along the length of the cannula. In some embodiments, the one or more apertures includes three apertures each disposed through the cannula wall thickness and placed at a location about 120 degrees off set from the two adjacent apertures such that about the perimeter of the cannula there are three apertures each having centers that are approximately equidistant from the center of the next aperture about the perimeter of the cannula. In some embodiments, the cannula tip is dome-shaped.

In some embodiments, at least one of the one or more apertures is an end hole defined by the dome-shaped tip. In various embodiments, the insulative layer is disposed on the exterior surface of the cannula but leaves conductive material of the cannula wall thickness and/or the exterior surface exposed at the one or more apertures. In some embodiments, the insulative layer is disposed on the surface of the cannula but leaves conductive material exposed about the one or more apertures on the exterior surface of the cannula. In some embodiments, the insulative layer is wrapped about the exterior surface of the cannula. In various embodiments, the insulative layer is heat shrunk about the exterior surface of the cannula. In some embodiments, the insulative layer is a coating disposed about the exterior surface of the cannula.

### Cosmetic System Features and Exemplary Embodiments

In part, in another aspect, the disclosure relates to a cosmetic tissue treatment system. The system includes a housing; a first pump disposed in the housing; a RF generator disposed in the housing; the RF generator configured to output treatment energy signals; a power supply disposed in the housing, the power supply in electrical communication with the first pump and the RF generator; a control system disposed in the housing, the control system in electrical communication with the RF generator and/or the first pump, the control system is configured to operate the RF generator in one or more of a pulsed RF energy mode and a continuous RF power mode; one or more probe connectors; and a tissue treatment probe having a length. The probe may be connected to one or more probe connectors, the probe includes a cannula includes a conductive material, the cannula having a wall thickness, the cannula includes a cannula wall, an inner surface, an outer surface, a proximal end, a length, a distal end, and a tip disposed is in a region of the distal end, the proximal end attachable to a handle, one or more apertures defined by the cannula wall, wherein application of an RF energy signal to an electrical contact of the probe generates a heat effect, wherein the heat effect is generated relative to a medium or tissue in fluid communication with the one or more apertures.

In some embodiments, the RF energy signal has one or more probe parameters, the one or more probe parameters selected to adjust or achieve the heat effect or a heat effect parameter. In some embodiments, the RF energy signal is a pulsed RF signal, wherein parameters of the pulsed RF signal results in heat confinement in an aqueous medium surrounding the cannula. In various embodiments, application of the pulsed RF energy signal causes formation of one or more heat bubbles in an aqueous medium surrounding the cannula. In some embodiments, the one or more probe parameters of the RF energy signal that are selected include at least one of pulsed RF, continuous wave RF, peak power of the RF energy signal, average power of RF energy signal, a repetition rate of RF signal, on-time of the RF signal and a RF frequency.

In some embodiments, the heat effect parameter is selected from one or more of the following: volume of heating, duration of heating, gradient of heating, amount of heat localization, heating efficiency for localized change of state or steam formation, heating efficiency for localized tissue effects, and RF efficiency for localized change of state or plasma formation, and heating efficiency for localized change of state or plasma formation. In various embodiments, a heating efficiency for localized tissue effects include one or more of more of localized tissue coagulation, localized tissue ablation or vaporization, localized mechanical disruption, localized thermo-mechanical tissue disruption, localized pressure disruption, or localized shockwave tissue disruption.

In some embodiments, the control system is configured to measure impedance on a real-time basis relative to a localized treatment region; and automatically adjust applied voltage of RF energy signal, using a control system, in response to a change in an impedance value measured with regard to the localized treatment region, wherein the applied voltage of the RF energy signal is adjusted in response to the change in impedance value such that the RF energy signal is delivered with a maximum peak power level. In some embodiments, the control system is configured to compensate for impedance variability during a cosmetic treatment by setting a pulse duration limit, and discontinuing treatment when the pulse duration limit is met. In various embodiments, the control system is programmed to terminate delivery of an RF energy signal during treatment when a measured reactance value is received or generated by the control system that is indicative of plasma formation.

In some embodiments, the control system is programmed to measure reactance, allowing plasma formation for a time period when a reactance value is measured that is indicative of plasma formation and terminating delivery of an RF energy signal upon the expiration of the time period. In some embodiments, a portion of the probe generates bubbles in response to delivery of RF energy to tissue or an aqueous medium in contact with one or more apertures of the cannula; wherein the control system provides audible or tactile feedback indicative of reaching a target per pulse energy delivery suitable for performing a cosmetic procedure. In various embodiments, the one or more probe parameters are peak power, average power, an energy per pulse and a repetition rate, wherein the pulsed RF signal has a peak power having a range of 200 W to 3 kW, an average power having a range of about 5 W to about 100 W, an energy per pulse range of about 1 J to about 5 J per pulse and a repetition rate range of about 5 Hz to about 100 Hz.

In some embodiments, the one or more probe parameters are peak power, average power, an energy per pulse, wherein the pulsed RF energy signal has a peak power having a range of about 200 W to about 3 kW, an average power having a range of about 0.1 W to about 10 W, an energy per pulse range of about 1 J to about 5 J per pulse and a repetition rate of about 0.5 Hz to about 10 Hz.

In some embodiments, the one or more probe parameters are peak power, average power, an energy per pulse, wherein the pulsed RF signal has a peak power having a range of about 200 W to about 3 KW, an average power having a range of about 1 W to about 60 W, an energy per pulse range of about 0.5 J to about 3 J per pulse and a repetition rate of about 1 Hz to about 60 Hz.

In various embodiments, application of the pulsed RF energy signal causes heat confinement in an aqueous medium surrounding the cannula. In some embodiments, the cannula further includes a port for communication with the first pump. In some embodiments, the first pump is an infiltration pump for delivery of tumescent fluid through the cannula. In various embodiments, the first pump is a suction pump for removal of unwanted tissue through the cannula. In some embodiments, application of at least one of the pulsed RF energy signal or the continuous RF power signal creates heat generation regions in an aqueous medium surrounding the cannula.

### Tissue Disruptor Features and Exemplary Embodiments

In part, in another aspect, the disclosure relates to a tissue disruptor. The tissue disruptor includes a hollow cylindrical structure having a wall thickness and sized to compliment a shape and one or more dimensions of one of the outer diameter or the inner diameter of a cannula employed to suction tissue; at least one disruptor aperture disposed through the wall thickness, the disruptor aperture sized to compliment dimensions of a cannula aperture disposed through a cannula wall thickness in a cannula employed to suction tissue and the disruptor aperture is positioned to align with a cannula aperture; and a resistor is disposed about an edge of the disruptor aperture, the resistor positioned to generate heat sufficient to thermally confine heating of an aqueous media surrounding the resistor.

In some embodiments, tissue disruptor further includes at least one gap in the continuity of the resistor disposed about the edge of the disruptor aperture. In various embodiments, the resistor is a thin film resistor heater. In some embodiments, the thin film resistor heater includes one or more materials, wherein the one or more materials includes at least one of Platinum, Tungsten, Nickel, Iron, Chromium, Nickel-chromium alloys, Iron-chromium alloys, or combinations of these. In some embodiments, the resistor selected to deliver between about 0.1 Joules and about 5 Joules of heating energy per aperture during electrical on-time that ranges between about 1 ms and about 10 ms.

In various embodiments, at least two ends of the resistor are capable of connection to a power supply. In some embodiments, the hollow cylindrical structure compliments the outer diameter of a cannula employed to suction tissue such that when the hollow cylindrical structure is assembled with a cannula the disruptor aperture aligns with a cannula aperture to form a single opening through both the cannula wall thickness and the disruptor wall thickness. In some embodiments, the resistor can generate heat sufficient to clear unwanted tissue from a cannula aperture in a cannula employed to remove unwanted adipose tissue.

In various embodiments, the resistor is configured to deliver RF energy configured to generate steam bubbles in a material in fluid communication with a cannula aperture in a cannula employed to remove unwanted adipose tissue, wherein a RF energy signal received by the resistor has one or more probe parameters, the one or more probe parameters selected to adjust or achieve the heat effect or a heat effect parameter. In some embodiments, the resistor delivers short electrical pulses near a cannula aperture in response to the probe parameters selected. In some embodiments, the resistor is electrically insulated from a cannula includes a conductive material. In various embodiments, the disruptor has a bore or cavity sized to slidably receive the cannula.

In part, in another aspect, the disclosure relates to a tissue disruptor and cannula assembly. The assembly includes a cannula employed to suction tissue, at least one cannula aperture defined by a cannula wall, the cannula wall having a cannula wall thickness; a disruptor includes a hollow cylindrical structure includes a disruptor wall having a disruptor wall thickness and inner dimensions sized to compliment the outer shape and dimensions of the cannula; at least one disruptor aperture defined by the disruptor wall, the disruptor aperture is sized to compliment all or a portion of the cannula aperture dimensions, the disruptor aperture having an edge defined by the disruptor wall; and a resistor disposed about the edge of the disruptor aperture, the resistor configured to generate heat sufficient to thermally heat a volume of an aqueous media surrounding the resistor. In various embodiments, the hollow cylindrical structure of the disruptor is sized and arranged to align the disruptor aperture and the cannula aperture, wherein the disruptor aperture aligned with the cannular aperture define a channel through both the cannula wall and the disruptor wall.

In many embodiments, the assembly further includes providing instructions to insert the cannula into the hollow cylindrical structure of the disruptor and to align the disruptor aperture with the cannula aperture. In some embodiments, the assembly further includes at least one gap in the continuity of the resistor disposed about the edge of the disruptor aperture. In various embodiments, the resistor is selected to deliver between about 0.1 Joules and about 5 Joules of heating energy per disruptor aperture during electrical on-time that ranges between about 1 ms and about 10 ms.

In some embodiments, at least two ends of the resistor are capable of connection to a power supply. In many embodiments, the resistor is selected to generate heat sufficient to clear unwanted tissue from a cannula aperture in a cannula employed to remove unwanted adipose tissue. In various embodiments, the resistor is selected to deliver heat sufficient to generate steam bubbles near a cannula aperture in a cannula employed to remove unwanted adipose tissue. In some embodiments, the resistor is selected to deliver short electrical pulses near a cannula aperture in a cannula employed to remove unwanted adipose tissue. In various embodiments, the resistor is electrically insulated from a cannula.

### Cosmetic Methods and Related Features and Exemplary Embodiments

In part, in one aspect, the disclosure relates to a cosmetic tissue treatment method. The method includes providing a tissue treatment probe includes a cannula includes a conductive material, the cannula having a cannula wall thickness, and having a proximal end, a length, and a distal end, a tip is in the region of the distal end, the proximal end is coupled to a handle, one or more apertures are disposed through the wall thickness; inserting the distal end of the probe into a region of tissue targeted for cosmetic treatment; and applying an RF energy signal to the cannula to generate a heat effect on a medium or tissue in fluid communication or adjacent the one or more apertures, wherein the RF energy signal that is applied has one or probe more parameters, the one or more probe parameters selected to adjust or achieve the heat effect or a heat effect parameter.

In many embodiments, the one or more probe parameters of the RF energy signal that are selected include at least one of pulsed RF, continuous wave RF, peak power of the RF energy signal, average power of RF energy signal, a repetition rate of RF signal, on-time of the RF signal and a RF frequency.

In some embodiments, the heat effect parameter is selected from one or more of the following: volume of heating, duration of heating, gradient of heating, amount of heat localization, heating efficiency for localized change of state or steam formation, heating efficiency for localized tissue effects, and RF efficiency for localized change of state or plasma formation, and heating efficiency for localized change of state or plasma formation.

In various embodiments, a heating efficiency for localized tissue effects include one or more of more of localized tissue coagulation, localized tissue ablation or vaporization, localized mechanical disruption, localized thermo-mechanical tissue disruption, localized pressure disruption, or localized shockwave tissue disruption. In many embodiments, the cannula is connected to a first pump. In some embodiments, the first pump is an infiltration pump for delivery of tumescent fluid through the cannula to adipose tissue targeted for cosmetic treatment, wherein the medium is an aqueous medium, the aqueous medium includes tumescence and unwanted adipose tissue.

In various embodiments, the method further includes a first pump, wherein the first pump is a suction pump for removal of at least a portion of an aqueous medium comprising tumescence and unwanted adipose tissue from the region of tissue targeted for cosmetic treatment through the cannula. In some embodiments, the tissue is at least one of dermal tissue, septa, and fibrous tissue.

In many embodiments, the application of the pulsed RF energy signal causes heat confinement in the aqueous medium surrounding the cannula to cause rapid vaporization of a portion of the aqueous medium present in the region of the aperture thereby forming one or more bubbles. In various embodiments, forming the bubbles is followed by collapse of one or more of the formed bubbles thereby contributing to breakdown of the adipose tissue in the aqueous medium.

In some embodiments, the cannula is connected to a first pump and the first pump is a suction pump for removal of at least a portion of the aqueous medium from the region of tissue targeted for cosmetic treatment through the cannula. In many embodiments, the pulsed RF energy signal applies pulsed RF energy that ranges from about 1 Joule per pulse to about 5 Joules per pulse and delivered at a pulse frequency that ranges from about 5 Hz to about 100 Hz. In various embodiments, the cannula is connected to a first pump and the first pump is a suction pump for removal of at least a portion of the aqueous medium from the region of tissue targeted for cosmetic treatment through the cannula, wherein the bubbles disrupt all or a portion of any blockage in the one or more apertures thereby reducing or avoiding interruption of aqueous medium removal through the cannula.

In some embodiments, the pulsed RF energy signal applies pulsed RF energy that ranges from about 1 Joule per pulse to about 5 Joules per pulse and delivered at a pulse frequency that ranges from about 0.5 Hz to about 10 Hz. In many embodiments, the method further includes measuring impedance on a real-time basis relative to a localized treatment region; and automatically adjust applied voltage of RF energy signal, using a control system, in response to a change in an impedance value measured with regard to the localized treatment region, wherein the applied voltage of the RF energy signal is adjusted in response to the change in impedance value such that the RF energy signal is delivered with a maximum peak power level.

In various embodiments, the method further includes compensating for impedance variability during a cosmetic treatment by setting a pulse duration limit, and discontinuing treatment when the pulse duration limit is met. In some embodiments, the method further includes measuring reactance and terminating the delivery of an RF energy signal during treatment when a reactance value is measured that is indicative of plasma formation. In various embodiments, the method further includes measuring reactance, allowing plasma formation for a time period when a reactance value is measured that is indicative of plasma formation and terminating the delivery of an RF energy signal upon the expiration of the time period.

In many embodiments, the method further includes initiating, promoting or causing formation of one or more heat bubbles in an aqueous medium surrounding the cannula and in fluid communication with the one or more apertures in response to application of the RF energy signal. In some embodiments, the method further includes generating bubbles in response to delivery of RF energy to tissue or an aqueous medium in contact with one or more apertures of the cannula; providing audible or tactile feedback indicative of reaching a target per pulse energy delivery suitable for performing a cosmetic procedure. In various embodiments, the RF energy signal is pulsed.

In part, in another aspect, the disclosure relates to a cosmetic tissue treatment method. The method includes providing a tissue treatment probe includes a cannula includes a conductive material, the cannula includes a cannula wall, a proximal end, a length, a distal end, and a probe tip, one or more apertures are defined by the cannula wall, wherein the cannula is connected to a suction pump; inserting the probe tip into a region of tissue targeted for adipose tissue removal and/or tissue heating; delivering tumescent fluid to the region of tissue targeted for adipose tissue removal, thereby forming an aqueous medium of tumescence and unwanted adipose tissue; and applying pulsed RF energy to the cannula to generate one or more heat effects on an aqueous medium or tissue adjacent the one or more apertures, wherein the one or more heat effects is at least one of: heat confinement, plasma formation, bubble formation, and localized heating.

In many embodiments, the pulsed RF energy signal applies pulsed RF energy that ranges from about 1 Joule per pulse to about 5 Joules per pulse and delivered at a pulse frequency that ranges from about 5 Hz to about 100 Hz thereby forming one or more bubbles followed by bubble collapse thereby contributing to breakdown of the adipose tissue in the aqueous medium prior to removal thereof through the cannula by application of suction using the suction pump.

In some embodiments, the pulsed RF energy signal applies pulsed RF energy that ranges from about 1 Joule per pulse to about 5 Joules per pulse and delivered at a pulse frequency that ranges from about 0.5 Hz to about 10 Hz thereby forming one or more bubbles, wherein the bubbles disrupt all or a portion of any blockage in the one or more apertures thereby reducing or avoiding interruption of aqueous medium removal through the cannula by application of suction using the suction pump. In various embodiments, the pulsed RF energy signal applies pulsed RF energy that ranges from about 0.5 Joule per pulse to about 3 Joules per pulse delivered at a pulse frequency that ranges from about 1 Hz to about 60 Hz, wherein the heat confined aqueous medium transfers heat to surrounding tissue includes one or more of dermal tissue, septa tissue, and fibrous tissue.

In some embodiments, the method further comprises an infiltration pump, wherein the cannula is connected to the infiltration pump and wherein the infiltration pump delivers tumescent fluid through the cannula to the region of tissue targeted for adipose tissue removal. In various embodiments, the one or more heat effects is heat confinement.

In many embodiments, the heat confinement is promoted in the aqueous medium surrounding the cannula, wherein the heat confined aqueous medium transfers heat to surrounding tissue comprising one or more of dermal tissue, septa tissue, and fibrous tissue. In some embodiments, the heat confinement is promoted in the aqueous medium surrounding the cannula and configured to initiate rapid vaporization of a portion of the aqueous medium present in the region of the aperture thereby forming one or more bubbles followed by bubble collapse thereby contributing to breakdown of the adipose tissue in the aqueous medium prior to removal thereof through the cannula by application of suction using the suction pump.

In various embodiments, the heat confinement is promoted in the aqueous medium surrounding the cannula and configured to initiate rapid vaporization of a portion of the aqueous medium present in the region of the aperture thereby forming one or more bubbles wherein the bubbles disrupt all or a portion of any blockage in the one or more apertures thereby reducing or avoiding interruption of aqueous medium removal through the cannula by application of suction using the suction pump. In some embodiments, the method further includes reducing clogging of material flowing through one or more apertures of the probe by selectively insulating the probe from an RF energy signal received by the probe and directing RF energy from the conductive material to the material.

Although, the disclosure relates to different aspects and embodiments, it is understood that the different aspects and embodiments disclosed herein can be integrated, combined, or used together as a combination system, or in part, as separate components, devices, and systems, as appropriate. Thus, each embodiment disclosed herein can be incorporated in each of the aspects to varying degrees as appropriate for a given implementation. Further, the various systems, probes, applicators, needle arrays, controllers, components and parts of the foregoing can be used with any suitable tissue surface, cosmetic applications, and medical applications and other methods and conjunction with other devices and systems without limitation.

These and other features of the applicant's teachings are set forth herein.

### BRIEF DESCRIPTION OF THE FIGURES

The patent or application file contains at least one drawing executed in color. Copies of this patent or patent application publication with color drawing(s) will be provided by the Office upon request and payment of the necessary fec.

Unless specified otherwise, the accompanying drawings illustrate aspects of the innovations described herein. Referring to the drawings, wherein like numerals refer to like parts throughout the several views and this specification, several embodiments of presently disclosed principles are illustrated by way of example, and not by way of limitation. The drawings are not intended to be to scale.

FIG. 1A is a schematic diagram of a prior art liposuction system that employs two separate liposuction systems, one Power Assisted Liposuction (PAL) and one ultrasound assisted liposuction system, together with a separate suction system and a separate RF assisted tightening system.

FIG. 1B is a schematic diagram of an integrated liposuction system that in a single unit offers infiltration, Power Assisted Liposuction (PAL), RF assisted liposuction, suction, and heating for invasive and/or minimally invasive treatment amongst other surgical and noninvasive applications according to an embodiment of the disclosure.

FIG. 2 is a schematic diagram showing an arrangement of user controls or inputs with a three pedal foot switch and representative mapping of controls to three user inputs/foot pedals according to an exemplary embodiment of the disclosure.

FIGS. 3a, 3b, and 3c are schematic diagrams that depict three different probes suitable for use with the systems and methods disclosed herein according to an exemplary embodiment of the disclosure.

FIGS. 4A-4E are schematic diagrams that depict three different probes suitable for use with the systems and methods disclosed herein according to an exemplary embodiment of the disclosure.

FIGS. 5A-5B are diagrams showing various RF-based probe embodiments or portions thereof according to an exemplary embodiment of the disclosure.

FIG. 5C shows an RF-based probe embodiment including a handle, a probe cannula with suction and a probe tip, a suction hose, and a wire according to an exemplary embodiment of the disclosure.

FIG. 5D is a diagram showing a portion of an RF-based probe according to an exemplary embodiment of the disclosure.

FIG. 5E is a schematic diagram that depicts liposuction and heating and tightening with a single probe for use in continuous RF power mode according to an exemplary embodiment of the disclosure.

FIG. 5F is a perspective view showing a probe tip of a liposuction cannula according to an exemplary embodiment of the disclosure.

FIG. 5G is a perspective view showing the probe tip of FIG. 5F applied with pulsed RF energy according to an exemplary embodiment of the disclosure.

FIG. 5H shows an RF-based probe embodiment including a handle, a probe cannula with suction and a probe tip, a suction hose, and a wire according to an exemplary embodiment of the disclosure.

FIG. 5I shows another view of the probe cannula with suction and a probe tip from FIG. 5H according to an exemplary embodiment of the disclosure.

FIG. 5J is a schematic diagram that depicts liposuction, heating and tightening, and clean up/finesse with a single pulsed RF probe and a footswitch according to an exemplary embodiment of the disclosure.

FIGS. 5K-5L are various perspective views showing a probe tip having six holes with three through hole according to an exemplary embodiment of the disclosure.

FIGS. 5M-5N are various perspective views showing a probe tip having an end hole that is defined by a sidewall or cylindrical housing of the probe tip to eliminate dead space according to an exemplary embodiment of the disclosure.

FIG. 5O is a perspective view of a tissue disruptor that can be added to a state-of-the-art cannula probe to avoid clogs forming in one or more aperture and/or in the cannula according to an exemplary embodiment of the disclosure.

FIG. 6A is a schematic diagram showing treatment of a target tissue area in various stages of a method of tissue treatment that includes tumescence infiltration, tightening, and volume reduction (i.e., liposuction) with a single probe and in continuous RF power mode via Steps 1, 2, and 3 according to an exemplary embodiment of the disclosure.

FIG. 6B is a schematic diagram showing treatment of a target tissue area in various stages of a method of tissue treatment that includes tumescence infiltration, volume reduction (i.e., liposuction), tightening via reticular dermis coagulation, and optionally a finesse step with a single probe and in a pulsed RF energy mode via Steps 1, 2, 3 and 4 according to an exemplary embodiment of the disclosure.

FIG. 6C is a schematic diagram including Steps a, b, c, and d that show a perspective view of a probe cannula in which an unenergized cannula is inserted into tissue between pulses, upon introduction of pulsed RF energy an aperture with high instantaneous current density creates localized heat around the aperture, bubble formation results from rapid vaporization of water, fat and/or tumescence, and the expansion and collapse of bubbles disrupts fat and the fat is sucked away according to an exemplary embodiment of the disclosure.

FIG. 6D includes schematic diagrams including Steps a, b, c, and d that show a perspective view of a probe 500 cannula creating a series of coagulation zones near the reticular dermis and/or at the dermis/fat junction according to an exemplary embodiment of the disclosure.

FIG. 7 is an exemplary graphical user interface (GUI) disposed on a console of a treatment system according to an exemplary embodiment of the disclosure.

FIG. 8A is a schematic diagram showing an applicator or probe that includes reusable handle and temperature monitoring portions according to an exemplary embodiment of the disclosure.

FIGS. 8B-8C are perspective views of two exemplary disposable probe tips according to an exemplary embodiment of the disclosure.

FIGS. 9A-9B are schematic diagrams showing side views of various probe designs or components thereof in one or more configurations according to an exemplary embodiment of the disclosure.

FIGS. 10A, 10B, and 10C are schematic diagrams showing side views of various probe designs or components thereof in one or more configurations according to an exemplary embodiment of the disclosure.

FIG. 11A is a plot showing power versus time and impedance versus time without clogging according to an exemplary embodiment of the disclosure.

FIG. 11B is a plot showing power versus time and impedance versus time with clogging according to an exemplary embodiment of the disclosure.

FIG. 12A is a plot showing peak power in Watts as the y-axis versus time in seconds as the x-axis and two separate approaches to pulsed RF energy mode where the higher peak power shorter duration pulses F1, F2, F3, and F4 are plotted in contrast to lower peak power longer duration pulses S1, S2, S3, and S4 according to an exemplary embodiment of the disclosure.

FIG. 12B is a plot of pulse to pulse impedance with impedance measured in Ohms on the y-axis versus time measured in seconds on x-axis according to an exemplary embodiment of the disclosure.

FIG. 12C is a plot of pulse to pulse voltage with voltage measured in arbitrary units on the y-axis versus time measured in seconds on the x-axis and these measurements correspond to the pulse to pulse impedance shown in FIG. 12B according to an exemplary embodiment of the disclosure.

FIG. 12D is a plot of pulse to pulse peak power over one second of treatment with peak power measured in Watts on the y-axis versus time measured in seconds on x-axis according to an exemplary embodiment of the disclosure.

FIG. 12E is a plot of pulse to pulse peak power over the course of a single undesirable pulse shown in FIG. 12D with peak power measured in Watts on the y-axis versus time measured in seconds on x-axis according to an exemplary embodiment of the disclosure.

FIG. 12F is a plot of pulse to pulse peak power over the course of a single desirable pulse shown in FIG. 12D with peak power measured in Watts on the y-axis versus time measured in seconds on x-axis according to an exemplary embodiment of the disclosure.

FIG. 13A is a plot of impedance measured in Ohms on the y-axis versus time measured in ms on the x-axis according to an exemplary embodiment of the disclosure.

FIG. 13B is a plot of reactance measured in Ohms on the y-axis versus time measured in ms on the x-axis according to an exemplary embodiment of the disclosure.

FIG. 14A is a plot showing an example of delivery of a preset energy density per unit volume by delivering pulses with variable on-time according to an exemplary embodiment of the disclosure.

FIG. 14B is shows the plot of FIG. 14A with the addition of impedance diagnostic pre-pulses as shown with a dashed line according to an exemplary embodiment of the disclosure.

FIG. 15 is a plot showing energy utilization efficiency, n, as a function of the normalized input energy density according to an exemplary embodiment of the disclosure.

FIGS. 16A and 16B are schematic diagram of annular electrodes (or aperture(s)) according to an exemplary embodiment of the disclosure.

FIG. 17 is a plot showing accumulated energy fractions versus on-time for uniformly heated variable diameter cylinders embedded in unheated aqueous media according to an exemplary embodiment of the disclosure.

FIG. 18 is a plot showing normalized input energy density versus the delivered RF energy per unit length of annular electrode for a range of uniformly heated variable diameter cylinders embedded in unheated aqueous media according to an exemplary embodiment of the disclosure.

FIG. 19A shows a normalized temperature rise distribution, in an example 3 mm diameter cannula and the surrounding tissue at the end of the heating pulse with variable on-time according to an exemplary embodiment of the disclosure.

FIG. 19B shows a normalized temperature rise distribution in an example, 3 mm diameter cannula and the surrounding tissue at the end of the heating pulse with variable on-time according to an exemplary embodiment of the disclosure.

FIG. 20 shows calculated peak normalized temperature rises in the center of the RF heated region at the end of the pulse for a range of heat generation thicknesses between 20 and 500 μm according to an exemplary embodiment of the disclosure.

FIG. 21 shows normalized input energy density as a function of the delivered RF energy per unit cannula length for a range of range of thicknesses of the RF heat generation region according to an exemplary embodiment of the disclosure.

FIG. 22 shows calculated peak normalized temperature rises in the RF heated toroidal like region at the end of the pulse for a range of heat generation toroidal diameters, d, between about 20 and about 500 μm according to an exemplary embodiment of the disclosure.

FIG. 23 shows normalized input energy density as a function of the delivered RF energy per unit length of annular electrode for a range of range of uniformly heated variable diameter cylinders embedded in unheated aqueous media according to an exemplary embodiment of the disclosure.

FIGS. 24A and 24B are cross-sectional views showing an exemplary RF dual liposuction and heating probe inserted into tissue and advanced to a treatment position according to an exemplary embodiment of the disclosure.

FIGS. 25A-25H are various views of probe portions that include a thermally insulated port and/or one or more support rings according to an exemplary embodiment of the disclosure.

### DETAILED DESCRIPTION

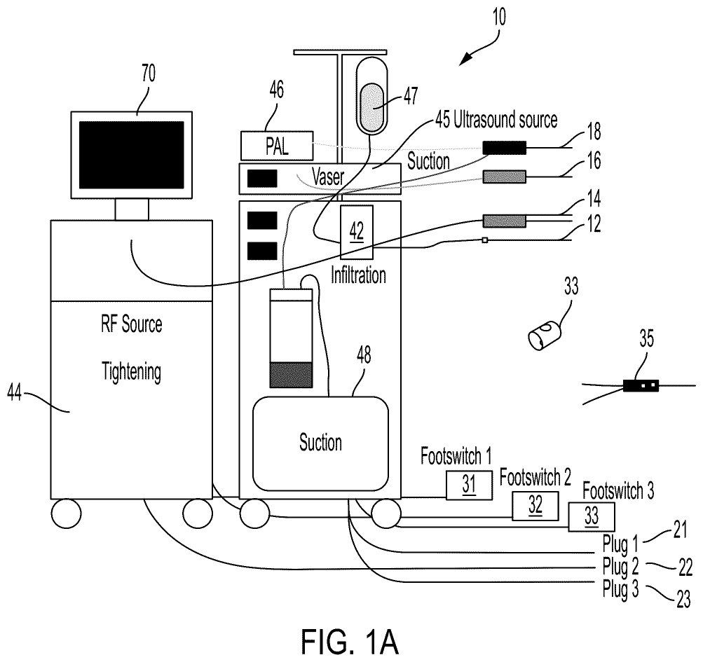

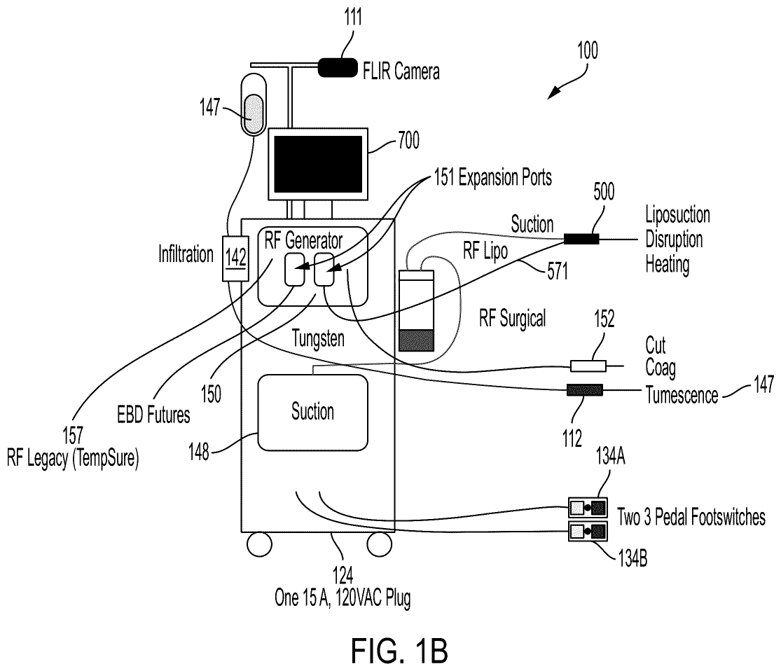

FIG. 1A represents a state-of-the-art plastic surgery suite suitable to offer both liposuction and tissue tightening. In some embodiments, the systems and methods disclosed herein may avoid the use of many different technologies requiring multiple systems and multiple means of control. As a result, various embodiments of the disclosure reduce disposable costs, surgical times, and the space and resources required to complete a treatment. More specifically, in some embodiments, the disclosed systems and methods improve upon existing state of the art liposuction procedures and systems 10, shown in FIG. 1A, in which the tissue is tumesced with one pump, an infiltration pump 42, and a first probe 12, tissue heating for the purpose of tightening is accomplished with a second probe 14, disruption of fat is accomplished with a third probe 16 and suction/resection is performed with a final fourth probe 18. In the existing systems and method of treatment each of these four probes may be supported by a different energy based or mechanically based platform, and each with a different wall service for electricity (i.e., 21, 22, 23) and a different footswitch 31, 32, 33 or other controller or method of operation. In some embodiments, the system 10 may be modified using a disruptor 33. The multiplicity of probes 12, 14, 16, and 18 may be replaced with one Radio frequency Assisted Liposuction probe 35 such as the various probes disclosed herein such as probe 500 and others. In some embodiments, one or more of the probes 12, 14, 16, and 18 may be modified with a disruptor 33. In various embodiments, the disruptor 33 may be any of the disruptors disclosed herein such disruptor 580.

Multiple separate systems are required to accomplish the multiple steps required in the procedure. More specifically, about three separate systems are required to accomplish the procedure according to the state-of-the-art. The prior art plastic surgery suite 10 depicted in FIG. 1A has a power assisted liposuction (PAL) system 46 in which a vibrating cannula breaks up fat cells prior to suction and this relatively fast rate of removal is suited to areas with a large amount of fat that require complete removal. The separate VASER system 45 is ultrasound assisted liposuction that is suited to body contouring and is reported to offer moderate skin tightening and finally a separate suction system is included to offer infiltration 42 whereby tumescence 47 solution is introduced and suction/aspiration 48 to remove the tissue being treated in the liposuction procedure. System 10 can be operated via Graphical User Interface (GUI) 70 to control the various described subsystems of the state-of-the-art system 10. Further, FIG. 1A depicts an RF source 44 that can be used for adipose tissue heating and for skin tightening. Various RF signals with one or more selected probe parameters may be used to achieve the various objectives and state changes described throughout the disclosure.

In part, the disclosure relates to various design enhancements and other advantageous systems, subsystem, and component embodiments. For example, as shown in FIG. 1B, a new surgical liposuction system 100 is disclosed that consolidates the necessary elements of a liposuction procedure that are accomplished with separate device systems, for example three separate devices, as depicted in FIG. 1A, into a single integrated system. Further, the single system 100 disclosed in association with FIG. 1B has faster fat removal relative to the state-of-the-art system 10 shown in FIG. 1A. Further the single system 100 in addition to faster fat removal has minimally invasive tissue tightening, an added capability in a single system relative to the state of the art.

The system 100 disclosed herein and shown in FIG. 1B, features Radio frequency Assisted Liposuction (RFAL), which employs RF to assist in the case and control of the rate of tissue removal as compared to PAL. Referring still to FIG. 1B, a probe 500 (e.g., a RF probe having a cannula with apertures defined in a cannula wall) and an RF generator 150 are designed to augment liposuction in a single system, but further still, the disclosed system can also offer a means to heat tissue with the same probe 500 thereby to achieve two different, desirable tissue effects with the same probe 500. Specifically, the single RF probe 500 can be used to heat, disrupt and remove fat tissue (e.g., adipose tissue). In various embodiments, the probe may also be referred to as a RFAL probe, or a liposuction probe, or a suction probe, and otherwise as disclosed herein.

In various embodiments, the probe includes a housing that defines various fluid transporting cavities, bores, volumes as well as apparatus, channels, holes, and slots. The probe includes a cannula that has a cannula wall in various embodiments. The probe may include one or more electrically conductive assemblies, regions, sections or other conductive surfaces or structures as disclosed and depicted herein. Various insulator and insulative materials may also be used in the probe such that only some conductive regions of the probe are in electrical communication with an RF source such as an RF generator. A given probe may also include various signal lines and cables or other conductive pathways and circuit elements to receive RF signals and generate a heat effect or otherwise heat a tissue and/or an aqueous medium or slurry of tissue. The heating performed by the probe is confined in various embodiments such that one or more parameters are selected such that the degree of heating of tissue and/or aqueous medium is limited to support targeted liposuction or other cosmetic treatments without heating other tissue such as muscle, veins, arteries, muscle, and other non-target tissues.

The RF probe 500 can traverse the tissue area being treated more smoothly than state of the art approaches to liposuction thereby lessening and/or avoiding arm fatigue associated with the procedure. The RF probe 500 provides an improved opportunity to control the application of energy as initially laid down in the tissue thereby enabling the practitioner who uses care to achieve the desired level of unwanted adipose tissue removal from a targeted area with precision thereby reducing the time required to complete the procedure as desired.

In some embodiments, faster fat removal is achieved by the disclosed system because the disclosed system can actively avoid clogging in the cannula caused by the removal of fat tissue, thereby avoiding downtime associated with clog removal remediation. Clogs and the required breaks in treatment to remove clogs from treatment probes occur routinely in state-of-the-art liposuction methods. In current practice, clogs are removed from liposuction probes in various way, including, by the practitioner stopping the liposuction procedure and then brushing the interior of the probe to remove the clog and/or by pulling off the suction and reversing the suction being applied to dislodge the clog. In part, the disclosure relates to a method of reducing clogging of a liposuction probe during various cosmetic procedures. The method of reducing clogging may include applying RF energy to a conductive region of the cannula of the probe such that the fat or other tissue that would otherwise clog a given aperture of the probe is reduced in size, volume, density, texture, consistency and other parameters as disclosed herein such that the individual broken down tissue elements pass through the aperture or are cause to release from the aperture before, during, or after the application of RF and a resultant heating effect such as localized or confined heating.

The RF probe 500 described herein can provide tissue tightening simultaneous with the fat removal step (e.g., during liposuction). The tissue tightening is via soft tissue coagulation and tightens relatively deep tissue (e.g., at a depth of a fiber septa network in a specific treatment region, which will vary based on the volume of unwanted adipose tissue content in a region to be treated) during the liposuction. Subsequent to aspiration of the unwanted fat via suction the RF probe 500 may provide tightening to tissue that is more superficial, e.g., at a depth of the tissue immediately below the dermis in a specific treatment region, which sometimes presents a challenge because the goal is to treat immediately below the dermis to even out the tissue appearance after treatment. The relatively deep tissue is at a depth that is deeper than the superficial tissue being treated according to the disclosure.

The system disclosed here includes a complete plastic surgery platform suitable for an operating room or surgical suite and such a platform combines the specific requirements of the RF generator 150 and its probes 500. Further, in addition to the RF generator 150, the platform may further support liposuction procedures by having an integrated suction aspiration vacuum pump 148 (or suction pump) and/or infiltration tumescence pump 142 in addition to the RF generator 150. The platform can further support PAL procedures; practitioners are commonly comfortable and capable doing PAL procedures and may utilize PAL alternatively or in addition to the RFAL procedure.

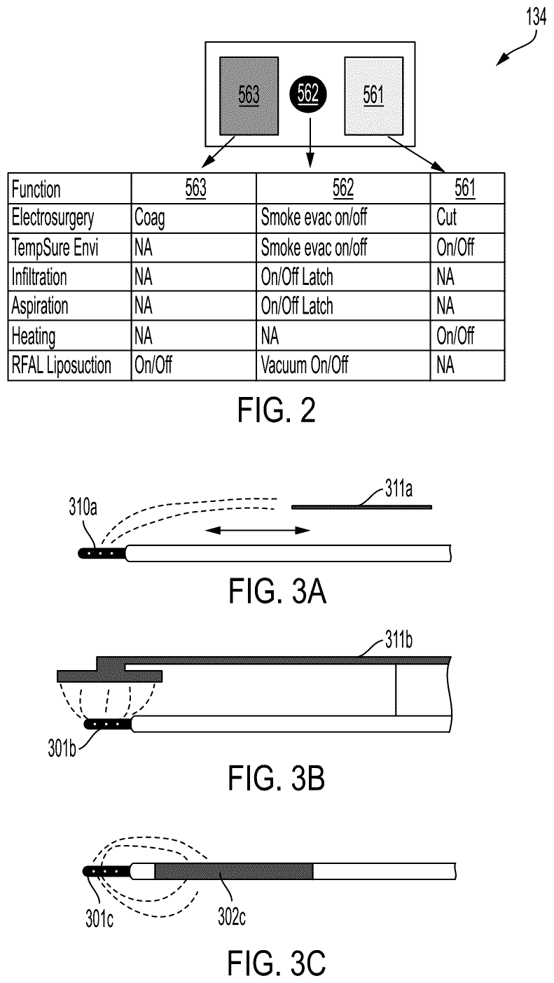

The patient procedure capability goals of this new RF platform may also include full surgical capabilities, non-invasive aesthetic capabilities and minimally invasive capabilities. FIG. 2, describes at least some of the breadth of procedures including and beyond RFAL that can be performed using the system disclosed herein. In one embodiment, referring to FIGS. 1B and 2, the breadth of possible procedures is controlled in the unit by using one or more 3 pedal footswitches 134 (e.g., two 3 pedal footswitches 134A, 134B) to accomplish a variety of treatment functions, and the breadth of procedures can include:
(1) Electrosurgery Function 152 (including Cut and Coagulation and optionally a blend of Cut and Coagulation. Such electrosurgery systems are commercially available, for example, via the TempSure system sold by Cynosure, LLC.
(2) tissue heating via probe 500 by delivering monopolar radio frequency energy to non-invasively heat the deep layers of the dermis without damaging the epidermis, encouraging new collagen formation. This technology increases the skin's temperature for a defined, therapeutic time to trigger a natural response in tissue (e.g., skin tissue) to create new collagen. Similar tissue heating systems are commercially available via the TempSure Envi handpiece sold by Cynosure, LLC, and this function is labeled TempSure Envi.
(3) Infiltration Function (e.g., infiltration of tumescent solution 147 via tumescent probe 112).
(4) Aspiration Function via probe 500 (e.g., vacuum/suction removal of tissue treated with or without RF).
(5) Heating Function via probe 500 (e.g., minimally invasive below the skin surface heating locally on at least a portion of the cannula (e.g., the tip), but without suction).
(6) RFAL Liposuction Function as described herein via probe 500.

The proposed treatment system may include one or more housings. In some embodiments, a RF generator 150 (optionally, for example, a one frequency RF generator or a two frequency RF generator), a peristaltic pump 142 and a suction pump 148 (e.g., a vacuum pump) may be part of the system and disposed in one or more housings. A peristaltic or infiltration pump 142 allows for infiltration, specifically, the administration of tumescence 147 to minimally invasive procedures and the suction pump 148 (e.g., vacuum pump) is required for tissue harvesting, specifically, aspiration, (a) tissue harvesting/tissue removal without RF or (b) tissue harvesting/tissue removal in concert with RF assisted liposuction.

The RF generator 150 can be employed with one or more frequencies (e.g., two frequencies) and the multiple temporal modes that can be used to deliver different invasive tissue effects which will be explained in greater detail. This same RF generator 150 can be used for standard electrosurgical cutting and coagulation via electrosurgery probe(s) 152 as well as driving monopolar and bipolar RF into skin for temperature-controlled tissue heating (e.g., surface tissue heating) via tissue heating RF handpieces 157 such as are sold under the tradename TempSure Envi (available from Cynosure, LLC in Westford, Massachusetts), for example. Such electrosurgical cutting and coagulation and surface tissue heating functions are disclosed in International Publication No. WO2019/157076A1 having International Application No. PCT/US2019/016883 entitled Methods and Apparatus for Controlled RF Treatment and RF Generator System and/or are commercially available under the tradename Tempsure® (available from Cynosure, LLC in Westford, Massachusetts).

Table 1 describes additional detail about the procedures achieved by using the pedal footswitch shown in FIG. 2.

TABLE 1

| | Platform Feature | Detailed Description |
|---|---|---|
| 1 | Improved Surgical | Compared to current electrosurgical performance, added |
| | CUT mode | power into high impedances for faster cutting. A given |
| | | system may include a current CUT mode for fine surgical |
| | | incisions and a high impedance CUT mode. |
| 2 | Aesthetic RF Heating | Capability to deliver RF power to the skin or mucosa via |
| | | monopolar handpieces with surface temperature regulation. |
| 3 | Infiltration capability | Pole for tumescence bag, peristaltic pump, tubing set, and |
| | | cannula to deliver tumescence prior to liposuction. |
| 4 | Standard suction for fat | No RF, allow standard suction assisted liposuction with any |
| | harvesting or standard | commercial probe. |
| | liposuction (i.e., PAL) | |
| 5 | Minimally-invasive | Fast, uniformly deposited coagulated zones near reticular |
| | tissue heating | dermis created with |
| | (tightening) | RFAL probes disclosed by the instant application. |
| 6 | RF assisted liposuction | Easier to pass through tissue, faster to reach clinical endpoint |
| | | goals with disclosed RFAL probe vs. power-assisted |
| | | liposuction. Optionally, the system can actively avoid |
| | | clogging of the liposuction probe (e.g., the cannula of the |
| | | probe) caused by removal of tissue. |

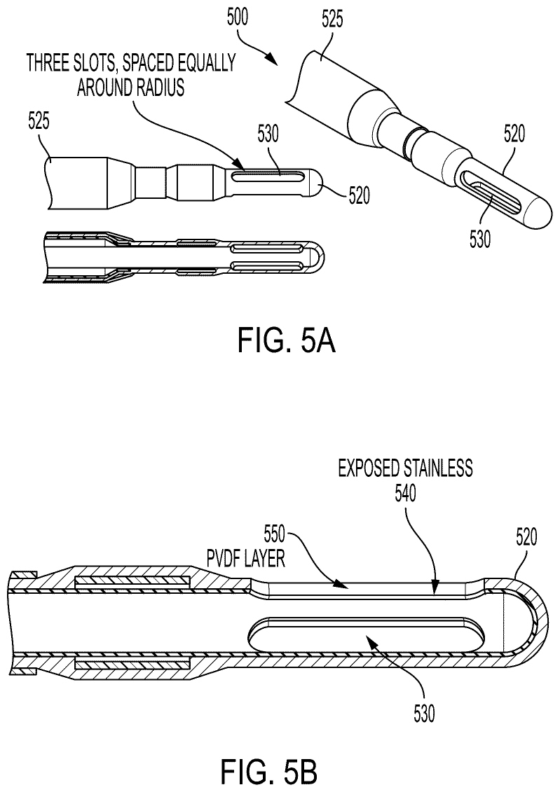

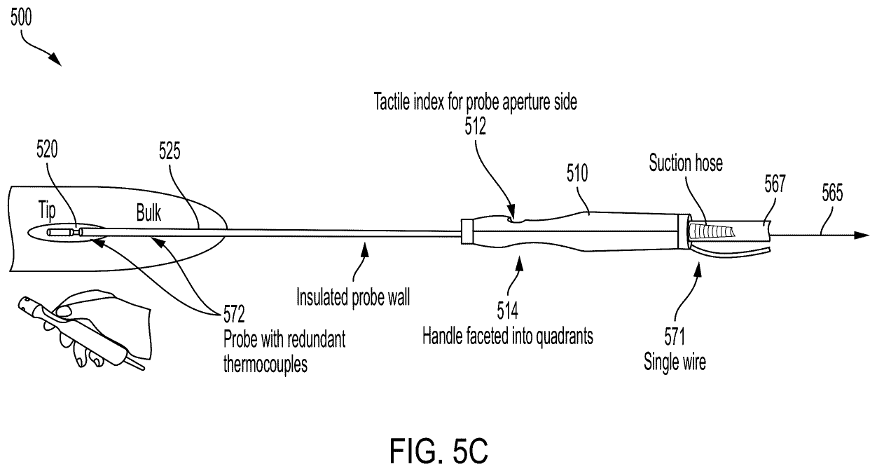

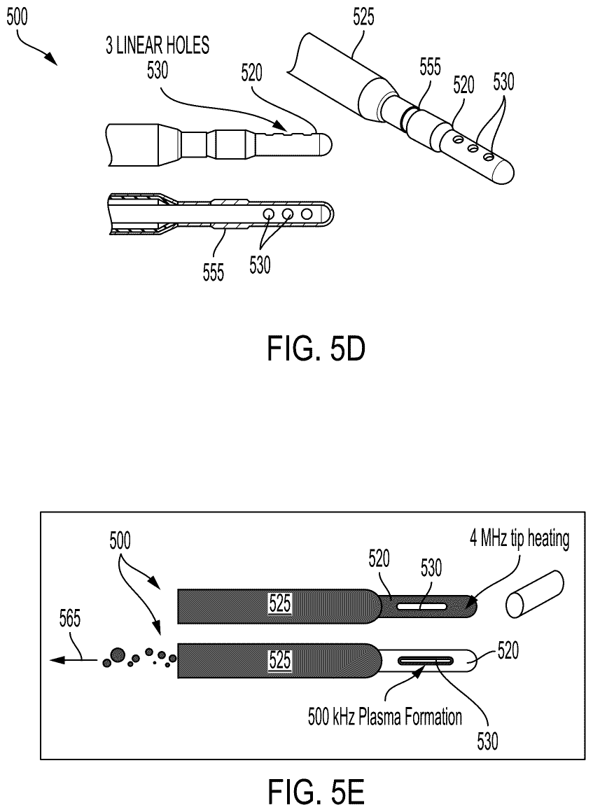

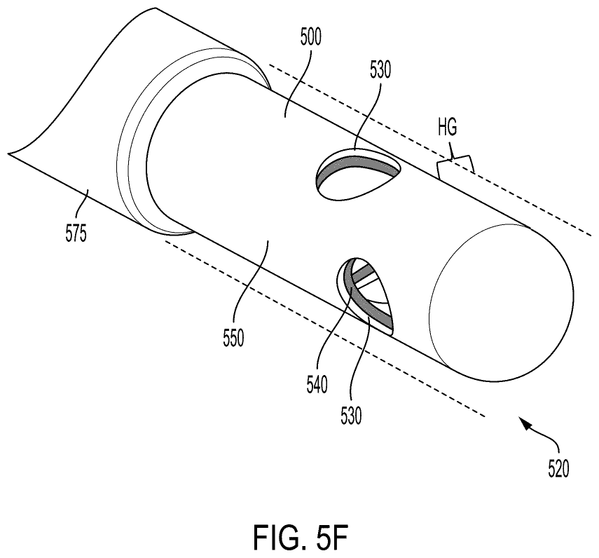

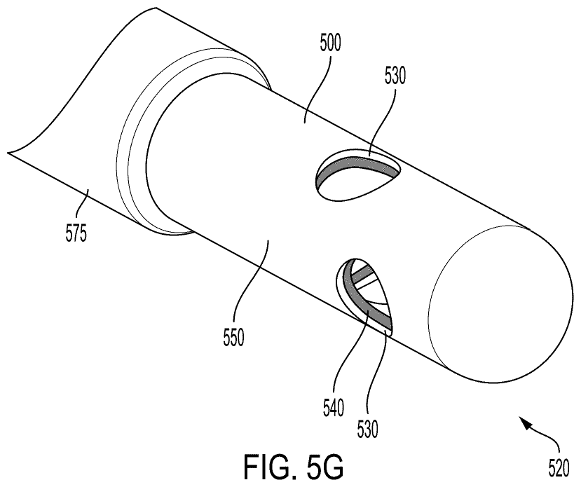

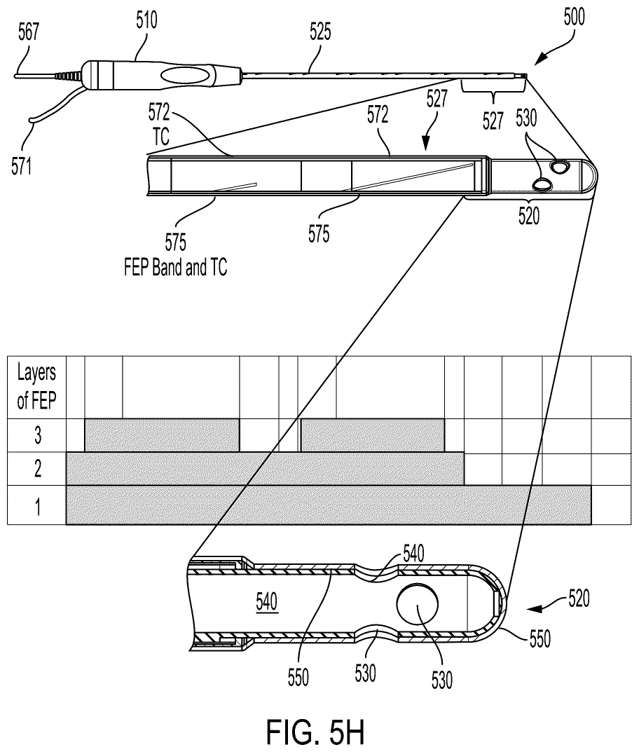

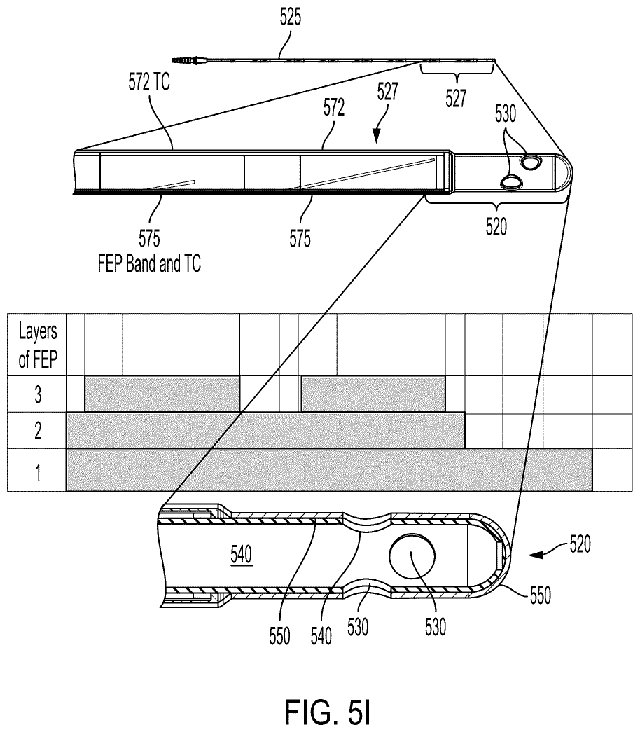

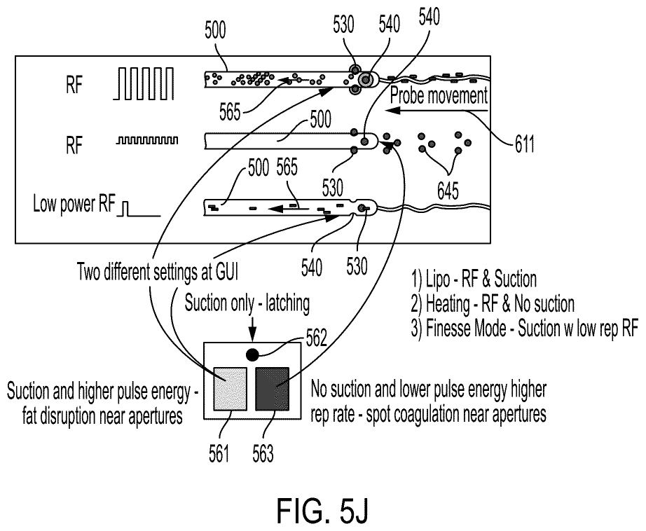

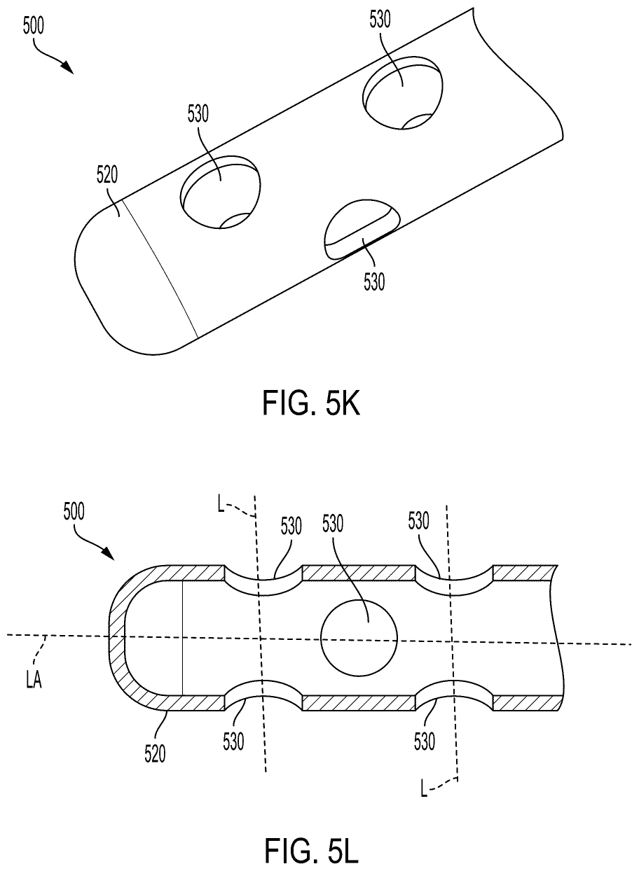

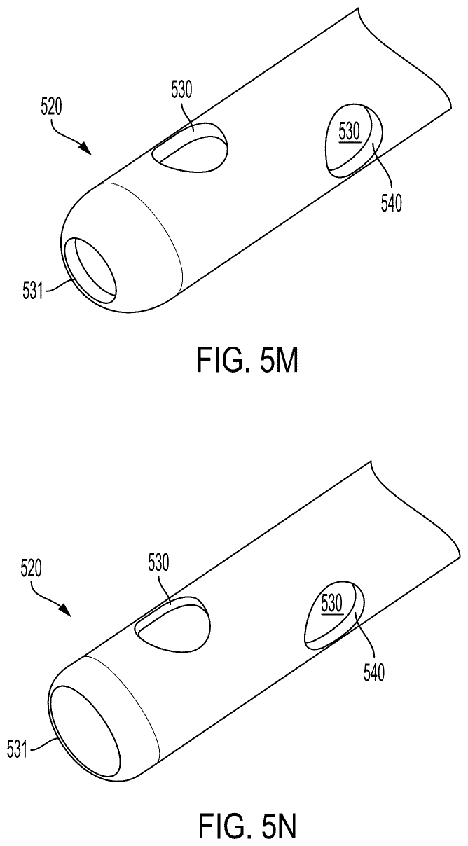

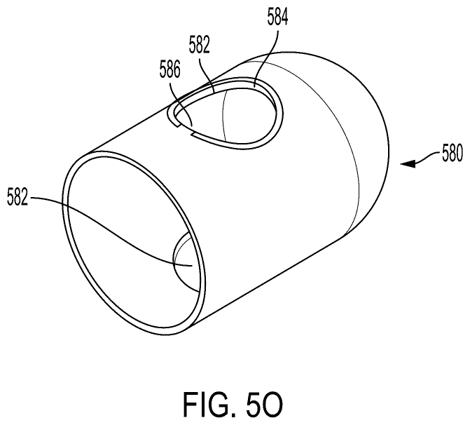

All of the procedures disclosed in Table 1 labeled (1) to (6) and including RFAL can be controlled with/using a single footswitch 134 or another controller. A layout of a given user controller, such as a footswitch 134 (e.g., a 3 pedal footswitch 134 having pedals 561, 562, 563 as depicted in FIG. 5J for example) control may be accomplished as is shown with reference to FIG. 2. Optionally, a GUI 700 on the console will indicate which foot pedal on the footswitch or other controller or user input may be activated for each of the modes selected: 1) Electrosurgery, 2) Aesthetic heating of surface tissue, 3) Infiltration is for the administration of tumescence, 4) Aspiration to activate the suction only, 5) Heating mode where tissue is heated locally to coagulation near the tip without suction and 6) the activation of RF and suction (RFAL). Further, optionally, a thermal camera 111 such as a FLIR camera is employed to monitor the temperature in the region of the probe being inserted into the area of tissue to be treated. Use of a thermal camera can provide a practitioner with assurance that the practitioner is aware of the actual temperature in the region of probe insertion and/or the tissue treatment region for the duration of the treatment.

The disclosed system shown in FIG. 1B uses a novel liposuction probe 500 coupled to a RF generator 150 configured as disclosed herein. The system architecture is such that the power supplies and RF generators 150 exist within the chassis and can provide power to expansion interface electronics expansion ports 151. The expansion ports 151 enable forward thinking future applicator development to plug into and be powered by the disclosed generator 150. Each expansion port 151 will have software and control algorithms that interface with to be designed future applicators whilst being powered by the disclosed RF generator system 150. Thus, market and consumer driven treatment capabilities are capable of expansion off of the existing RF generator 150 with the RF power of the disclosed generator providing the limiting factor rather than the hardware, software and controls that are initially built therein. Accordingly, exchangeable expansion can be made integral to the generator design and the overall system design. Further, the expansion ports can control the energy to the patient and serve as the communication bus between the handpiece (or the part applied to the patient) and the console. In this way, future handpieces and probes can be supported by a new expansion port box whereby the power from the console can power newly developed handpiece(s) or probe(s). The expansion ports in the unit enable future expansion of treatment capabilities without requiring additional separate systems. Floor space in surgical suites and operating rooms is at a premium. Building future expansion into the disclosed treatment system will obviate the need for additional bulky and space demanding systems in practitioners' offices. For example, while the disclosed probes/handpieces rely on temperature feedback, a future design may involve acoustic feedback (e.g., audible sounds indicating bubble formation informing that the correct pulse energy level is being applied to the treatment area) or force feedback (e.g., force feedback from the probe that indicates that the RF level should change and/or increase due to the presence of physical resistance indicative of fibrous tissue in the treatment region). This is possible because this single system is designed to accept and integrate an expansion box (see, e.g., expansion ports 151) with new system software without requiring a redesign of the console. For example, one expansion box could house a future laser for low level light therapy while being powered by the original console. In this way, practitioners can enhance their practices with future add on technology capabilities without requiring additional demand on the electrical service (e.g., only one outlet plug 124 may be required to power the system 100) and without appreciable changes to the demand in floor space in their various office treatment suites. Further, in contrast to the state-of-the-art system shown in FIG. 1A features multiple separate systems sourced by separate vendors to accomplish the multiple steps required to accomplish a liposuction and skin tightening procedure, the single system disclosed in accordance with FIG. 1B can simplify maintenance, because a single vendor may be the source of supplies, repairs, training and/or marketing materials.

### Monopolar Probe Features and Embodiments

In various embodiments, the systems and methods support monopolar and bipolar application of the RF via the liposuction probe shown and described in FIG. 1B. Several probe concepts were considered both with monopolar and bipolar applications of RF. For many or most or all embodiments, a monopolar approach is more favorable.

An explanation of the different polarity modes can be shown in FIGS. 3a, 3b, and 3c. A monopolar probe using a remote return pad normally on a flank, back or abdomen is shown in FIG. 3a. The probe of FIG. 3a may be compared with another probe arrangement as shown in FIG. 3b where the return pad follows the cannula on the upper surface of the skin. In another similar manifestation, the remote return pad could be a conductive glove worn on the practitioner's non-dominant hand that the practitioner normally follows the probe tip above the point of treatment. In contrast, with a bipolar cannula/probe as shown in FIG. 3c both electrodes are on the invasive part of the cannula. Use of a bipolar probe remains a viable option but adds complexity to the invasive probe by having two isolated electrical conduits along the length of the invasive cannula.

FIGS. 3a, 3b, and 3c respectively depict three different probes. FIG. 3a shows an exemplary monopolar probe embodiment that includes an active electrode at the tip of a moving cannula 301a and the return electrode 311a is stationary and fixed to the patient's body (body not depicted). FIG. 3b shows another exemplary monopolar probe embodiment that includes a return electrode 311b at the surface of the skin that follows the active electrode 301b below it within the skin (body/skin not depicted). FIG. 3c shows an exemplary probe embodiment having a bipolar arrangement that includes two separate electrodes on the single invasive cannula where a first electrode 301c is at the distal end and a second electrode 302c is adjacent the first electrode.

The monopolar probe construction of FIG. 3a is favorable and has various advantages as outlined below.
1) Ease of use, because with a monopolar approach there is no need to have an upper electrode skip or glide along the surface. In contrast, with a bipolar approach, the electrode needs to be in constant contact with the skin to complete the RF circuit. For some invasive devices, a bipolar electrode arrangement may include a heated needle tip within the skin/fat layers and a second surface electrode that is paired with that electrode in a fork-like manner. In such a bipolar arrangement the surface electrode stays on the skin surface whereas the needle is under the skin surface.
2) A monopolar probe is a simpler design with a lower cost of goods (COGS) as compared to a bipolar probe.
3) Heating of upper skin surface electrode, as would occur via a bipolar approach, is expected to be insufficient to achieve meaningful clinical effect for skin surface heating whereas the monopolar probe will offer sufficient heating and coagulation of the target reticular dermis and will preserve safe skin temperatures at the skin surface to provide a desirable clinical endpoint. Contrariwise, bipolar arrangements constrain the energy delivered to the maximum temperature achieved at the surface return electrode. High energy pulses can be applied with a monopolar approach since the return current distribution of the RF will be through a greater volume of tissue. In a monopolar arrangement the delivery of high energy pulses to deep dermis will not significantly raise the surface temperature thereby avoiding unwanted increases in skin surface temperature.

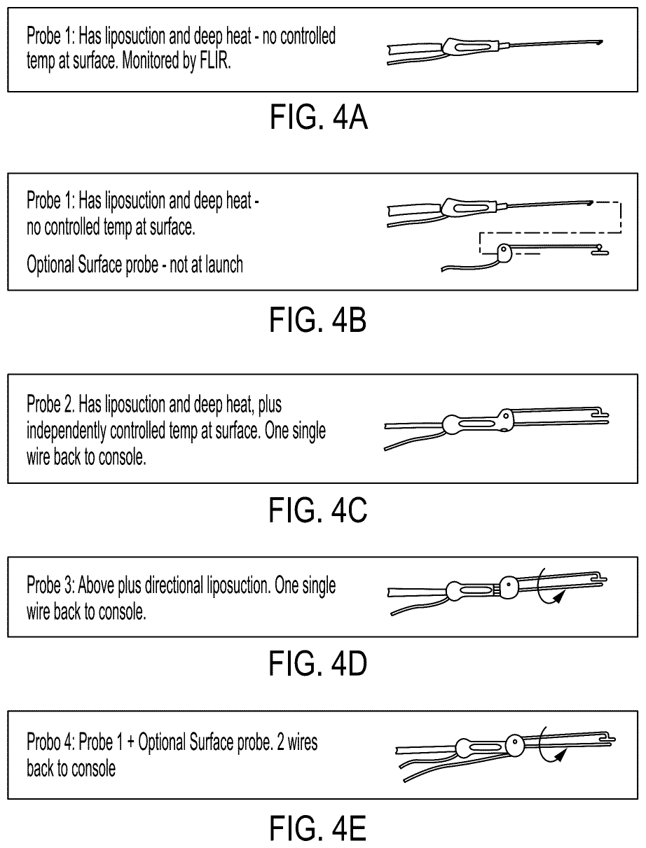

A range of optional monopolar probe designs is described in concert with FIGS. 4A-4E. In various embodiments, other probe designs may be used or modified in accordance with the disclosure. Probe 1 depicted in FIG. 4A is the simplest design of the ones depicted. It is a monopolar probe which relies on a return electrode to complete the RF circuit. This probe accurately measures the temperature at depth in the tissue but does not control the temperature at the surface. Through the careful, real-time monitoring of the temperature of tissue at depth, one can predict the surface temperature and stay within a safe limit while providing a desirable clinical endpoint through heating the upper skin surface.

FIG. 4B adds the potential of a separate surface temperature monitor that follows the Probe 1 tip, but hovers above the tip on the skin's surface. FIG. 4B shows the ability to add the surface temperature monitor to Probe 1 depicted in FIG. 4A as an option with an additional cable to connect back to the console.

Probe 2 depicted in FIG. 4C integrates the optional attachment of 4B with the Probe 1 from FIG. 4A such that surface temperature measurement is integrated into one probe with one wire leading back to the console. Thus, the monopolar probe relies on a return electrode to complete the RF circuit and the probe accurately measures the temperature at depth in the tissue while the surface temperature monitor hovers on the skin surface above the probe tip. The probe tip may be used together with the surface temperature monitor to achieve a safe limit while providing a desirable clinical endpoint through heating the upper skin surface.

FIG. 4D depicts Probe 3, which is similar to Probe 2, but additionally allows for rotation of the handpiece so that directional liposuction can be supported. This is only applicable to non-radially symmetric cannula aperture geometries that would favor liposuction to one side of the probe.

Probe 4 depicted in FIG. 4E combines the optional accessory of FIG. 4B and the Probe 1 of FIG. 4A, with the change in Probe 1 to additionally allow for rotation of the handpiece so that directional liposuction can be supported. This is only applicable to non-radially symmetric cannula aperture geometries that would favor liposuction to one side of the probe.

### Probe Construction Features and Embodiments

For some probe designs, RF is used to heat tissue with a surgical probe via a minimally invasive approach to achieve tissue tightening via soft tissue coagulation. By increasing the electrode area, one lowers the current density and subsequently achieves a gentler heating of tissue rather than vaporizing tissue which occurs at where there is a relatively high current density, for example, where the available electrode area is limited, for example, on the edge of a scalpel.

For a probe of about 3 mm in diameter and a coated tip 10 mm long, and ignoring apertures for the time being, there is approximately 60 mm² of area to conduct current to the surrounding tissue. Taking an average current of 1 A for example, 1 A will be distributed over 60 mm²=0.017 A/mm². This is considered a low current density compared to the edge of a scalpel blade where that same 1 A may be applied to an area 0.5 mm thick and 5 mm long=0.4 A/mm². The low current density will heat tissue without necessarily getting to phase changes such as that achieved at high current densities where one or more of steam bubbles, vaporization, plasma, and carbonization of tissue may occur.

Alternatively, the heating could be achieved through temporal manipulation of the power to achieve different tissue effects. For example, whereas high energy, short pulses (high power) might be optimized for tissue disruption and well suited for liposuction, the same frequency of RF could be temporally adjusted to deliver lower energy pulses at longer pulses (low power) and be optimized for localized heating and coagulation near the probe tip.

An invasive probe may be manufactured that operates with relatively lower current density heating properties (or only such properties) and because such an invasive probe does not require a suction lumen, the diameter of the probe can be quite small (sub-millimeter is possible). A smaller probe diameter is generally attractive to plastic surgeons performing minimally invasive cosmetic procedures or other invasive procedures. The RF frequency is not necessarily specific to this design-almost any frequency will work if the only goal is heating without vaporization, thus providing a relatively low current density. While the probe may feature an insulative layer or coating, the insulative layer or coating is not a requirement; in fact, in some embodiments, the absence of coating, e.g., using uncoated stainless steel, may also be employed for the heating mode.

The desired RF platform of the disclosure herein features a minimally invasive probe that can address both tissue tightening and tissue removal (e.g., adipose tissue) and be combined into one single probe, thus providing two functions in a single probe. There are various methods of optimizing liposuction and various methods of achieving tissue heating. The disclosed system 100 shown in FIG. 1B uses one or more disclosed liposuction probes 500 coupled to a RF generator 150 that can be used in continuous operation mode and/or in pulsed operation mode. References to stainless steel herein may also be generalized to include suitable conductive materials such as electrically conductive metals, alloys, and other structures with conductive pathways.

Use of the system disclosed in FIG. 1B in pulsed mode is advantageous, because it is possible to achieve RF assisted liposuction, deep tissue tightening and superficial tissue tightening, while actively avoiding cannula clogging. The system in pulsed mode delivers thermally confined heating to the tissue in the region of the probe 500 cannula, more specifically, the system in pulsed mode delivers thermally confined heating to the tissue adjacent to the aperture of the RF annular electrode. The precision associated with pulsed mode RF assisted liposuction avoids deficiencies associated with non-thermally confined approaches to energy delivery including continuous mode RF assisted liposuction.

Use of the system 100 disclosed in FIG. 1B in continuous operation mode has some limitations. Specifically, while it was expected that use of one or more of the disclosed RF probes 500 for liposuction in continuous operation mode would provide advantages including improved treatment speed, thorough treatment of adipose tissue in a given liposuction treatment region, amongst others, continuous mode RF assisted liposuction causes thermally unconfined heating with variable heat generation that is dependent upon the local tissue impedance. The region with the largest uncontrolled heat generation caused by thermally unconfined heating of continuous mode RF assisted liposuction can result in charring of tissue on the cannula. The thermally altered treated tissue oftentimes results in a clog of the cannula that terminates the ability for tissue to flow through the cannula for aspiration. Thus, without real time power feedback control and impedance detection of the localized tissue in the region where the probe 500 travels, reliable and/or consistent clinical results are difficult to achieve. The charring on the cannula and/or the clog of the cannula will compromise the probe 500 for use to complete a procedure without complex clog removal and/or render the probe unusable requiring disposal of the probe. Use of the system 100 disclosed in FIG. 1B in pulsed mode avoids the need for real time determination of impedance of the tissue in the region where the probe 500 travels and pulsed mode also avoids the need for real time power feedback control. Pulsed mode delivers thermally confined heating to the tissue and/or aqueous medium adjacent to the aperture of the RF annular electrode of the probe 500, thus pulsed mode delivers heating with precision. Accordingly, continuous operation of the system disclosed in FIG. 1B is best employed for deep tissue tightening and superficial tissue tightening and not for liposuction (e.g., in the absence of suction/vacuum).

### RF System Continuous Mode

FIGS. 1B, 5A-5E and 6A are employed to discuss use of the disclosed system 100 of FIG. 1B in continuous RF power mode. While the probes described in FIGS. 5A-5D are described in detail here for use in continuous RF power mode, the described probes 500 may also be employed in a pulsed RF energy mode or alternating between continuous RF power mode and pulsed RF power mode.
1) In continuous mode, the RF generator 150 is able to operate one distinct frequency in different temporal modes or two distinct frequencies (e.g., 500 kHz and 4 MHz) to seek two different effects, liposuction or tightening via tissue coagulation (e.g., soft tissue coagulation). In one embodiment, the system 100 described in FIG. 1B operates with each of the two separate RF frequencies with two RF frequency generators being present in a single enclosure (e.g., in a single console). This configuration also allows for two distinct temporal modes using one or two different frequencies to achieve two different clinical effects.

### For Liposuction:

2) It was expected that RF probes 500 disclosed in FIGS. 5A-5E would be used for liposuction in continuous operation mode to provide fast fat removal with less fatigue using radio frequency assisted liposuction (RFAL), at 500 kHz. The range of about 100 to about 1000 kHz or from about 350 kHz to about 750 kHz or about 500 kHz employed depending on the dielectric properties of the probe tip. Generally, where the frequency is lower (e.g., closer to 100 kHz) a given insulator employed is less likely to conduct current. In this way, where a higher frequency is employed (e.g., closer to 1000 kHz) the same given insulator is more likely to conduct current. The goal during fat removal is to avoid current leakage along the insulated portions of the probe thus a dielectric that can withstand frequency in the range from about 100 to about 1000 kHz without leaking current. The better the insulator (e.g., the higher the dielectric constant) that avoids current leakage will then concentrate the current about the aperture(s) of the cannula facilitating RFAL. It was found, however, that RF assisted liposuction in continuous mode causes thermally unconfined heating with variable heat generation that is dependent upon the local tissue impedance. This can result in charring of tissue on the cannula and/or a clog of the cannula that terminates the ability for tissue to flow through the cannula for aspiration rendering the probe unusable requiring disposal of the probe.

### For Heating:

3) In continuous RF power mode operation, referring still to FIGS. 5A-5E, fast tissue coagulation (tightening) of the reticular dermis, can occur at 500 kHz. The range of about 100 to about 1000 kHz or from about 350 kHz to about 750 kHz or about 500 kHz may be employed depending on the dielectric properties of the probe tip. The goal during heating for coagulation of the reticular dermis is to promote small, coagulated zones repeatedly throughout the plane immediately under the dermis. The difference between the coagulation mode (e.g., tightening) and the liposuction mode is that the pulse energy and power are reduced to only create high current densities sufficient to coagulate tissue and not form vaporization. An alternative heating method exploits a different RF frequency and its interaction with the frequency-dependent dielectric coating.

In continuous RF power mode, referring still to FIGS. 5A-5E, fast coagulation (tightening) of the reticular dermis, occurs at about 4 MHz. The range of from about 1 MHz to about 10 MHZ, from about 2 MHz to about 8 MHz, from about 1 MHz to about 4 MHz can be utilized for coagulation where one may intentionally choose higher frequency RF in order to better pass through the dielectric and enable more even and well distributed current density and heating about the coating's surface. The goal during heating for coagulation of the reticular dermis is to promote even current leakage along the insulated portions of the probe thus a selected dielectric that can withstand frequency in the range from about 100 to about 1000 kHz without leaking current and may enable current leakage when subjected to higher frequencies, e.g., in the range of from 1 MHZ to about 10 MHz, 1 MHz to 4 MHz. Referring now to FIG. 5A, the RFAL probe 500 is designed to concentrate an RF-generated high current density at the perimeter of the suction port. The one or more suction port is located at the one or more apertures 530 present at the probe tip 520. The frequency to achieve this high current density at the aperture perimeter can also vary but is typically configured in response to some physical properties of the probe tip. Referring still to FIG. 5A and also to FIG. 5E, handle 510 is connected to the probe shaft 525 and the probe shaft 525 can be thermally insulated or not. Tip 520 of the RFAL probe 500 is coated and the coating has dielectric properties. Specifically, the coating of the probe can be made from or contain Fluorinated Ethylene Propylene (FEP) or be wrapped in Teflon heat shrink. There are alternative electrical insulators that can be applied to the cannula shaft such as polyolefin, olefin, fluoropolymer (such as FEP, PTFE or Kynar), PVC, neoprene, silicone elastomer or Viton.

Preferably, the dielectric material or coating on the body of the probe (e.g., the tip 520 and/or the shaft 525) has dielectric properties that are insulative for both the fat removal frequency (e.g., 500 kHz) and the heating frequency (e.g., 200 kHz-10 MHz, or 350 kHz-7 MHz, and 500 kHz-4 MHZ). The dielectric properties are selected or chosen such that if active RF is applied to the stainless-steel cannula during the fat removal phase, there is little to no current leakage through the body (e.g., the tip 520 and/or the shaft 525) of the probe due to the properties of the dielectric material that is employed. Instead, the RF is concentrated about the perimeter of the one or more apertures 530 that are employed as suction ports during fat removal procedures. In various embodiments, probe shaft may also refer to probe cannula and vice versa.

Referring now to FIGS. 5A, 5B and 5E, the tip of probe 520 is at the distal end of probe shaft 525 (or cannula) as shown and there are three apertures 530 that are spaced equally about the diameter of the tip 520 of the probe. These apertures 530 in the probe tip are the point of entrance for the fat and the location through which the suction is applied to the unwanted adipose tissue during treatment. Hence, each of the apertures 530 can act as a suction port during fat removal treatment. Here, the conductive material 540 of the probe tip 520 is covered with an insulative coating 550. In the example shown in FIG. 5B, the conductive material 540 has a Polyvinylidene Fluoride (PVDF) coating, which has unique dielectric properties vs the stainless steel and FEP heat shrink. The dielectric properties of PVDF allow for the 500 kHz current to be concentrated to the exposed stainless steel located at the perimeter of the apertures 530, thus holding off leakage though the larger probe tip 520 area during fat removal treatment. At 4 MHz, the unique properties of PVDF allow the current to pass more easily through the coating. The area of the probe tip 520 aperture(s) 530 where the stainless steel is exposed (e.g., is not covered with the dielectric coating) represents the area of the probe 500 that generates high current density to assist in liposuction at, for example, 500 kHz. The PVDF coated area of the probe tip 520 represents the heating area for heating the upper layers of skin tissue (e.g., targeting the reticular dermis) when 4 MHz of current is applied to the probe (FIG. 5E). The insulating coating 550 on the probe tip 520 a range in thickness from about 0.000001 mm to about 10 mm in some embodiments. A given probe or cannula of the probe may include or be made from various conductive materials such as metals. In some embodiments, the conductive material is stainless steel. Various conductive materials suitable for receive and transmitting an RF energy signal may be used.

Referring now to FIG. 5E together with FIGS. 5A and 5B, where the RF generator is operated at 500 kHz, the insulative coating or layer 550 on the probe tip 520 and the FEP on the probe cannula shaft 525 are both insulative and conduct very little RF current. The dielectric properties of the insulative layer of PVDF do not allow conduction of the RF at this frequency (e.g., 500 kHz). Various insulative layers 550 may be used such as PVDF and others may be used. The current density provided by the 500 kHz is concentrated to the exposed conductive material 540 at the perimeter of the one or more apertures 530. Applicant believes that the RF concentrated about the perimeter of the one or more apertures 530 forms a concentrated current density that rapidly heats a small volume about the perimeter of the apertures 530.

In some embodiments, this rapid heating results in vaporization and heat and bubble formation to disrupt the tissue as suction is applied, thus expected to augment the liposuction process. The rapid heating can lead to vaporization of fluids and even plasma—this immediately breaks down tissue adjacent to the aperture. The RF-assist is intended to speed up a tedious operation and lyse immediately adjacent tissue into small pieces (e.g., decompose) or into liquid. Referring still to FIG. 5E, when suction is introduced through the hollow cannula of the probe 500, the lysed tissue 565 (e.g., liquefied, broken down or decomposed tissue) is drawn into the cannula without delay (e.g., immediately) and it was expected that the potential of any lysed tissue 565 clogging the cannula of the probe 500 would thereby be reduced and possibly avoided. However, as described herein, RFAL in continuous RF power mode resulted in unexpected charring and clogs in the probe cannula rendering the probes unusable prior to completion of the treatment. As mentioned, continuous RF power mode power is applied to the tip and results in high current densities which translates into heat. The tip of the stainless-steel probe is heated and passes through tissue more easily than an unheated probe. This is an added benefit to the application of RF to aid in the removal or heating of tissue in this manner.

Referring still to FIG. 5E together with FIGS. 5A and 5B, when the probe 500 is operated at 4 MHZ, the dielectric properties of the coating 550 on the cannula 525 and the tip 520 of the probe (e.g., the PVDF coating 550) allows the RF to pass and create a uniform current density on the cannula 525 and tip 520 of the probe relative to the area of the PVDF or in the region of the probe cannula 525 and/or tip 520 that has the coating 550. The area of the probe cannula covered by coating 550 of the probe cannula 525 and tip 520 (specifically, the probe cannula 525 and tip 520 diameter and length having the insulative coating 550 as well as the thickness or density of the insulative coating 550) and the RF frequency can be adjusted to create a wide range of tissue effects: (a) from a high current density vaporization or plasma for liposuction (not desirable for tissue heating) at 500 kHz (FIG. 5E) to (b) a low current density which can controllably heat tissue and is desirable to achieve tightening effects in tissue via soft tissue coagulation (e.g., tightening of the reticular dermis via soft tissue coagulation) at 500 kHz (FIG. 5E) or (c) a low current density which can controllably heat tissue and is desirable to achieve tightening effects in tissue via soft tissue coagulation (e.g., tightening of the reticular dermis via soft tissue coagulation) at 4 MHZ (FIG. 5E).

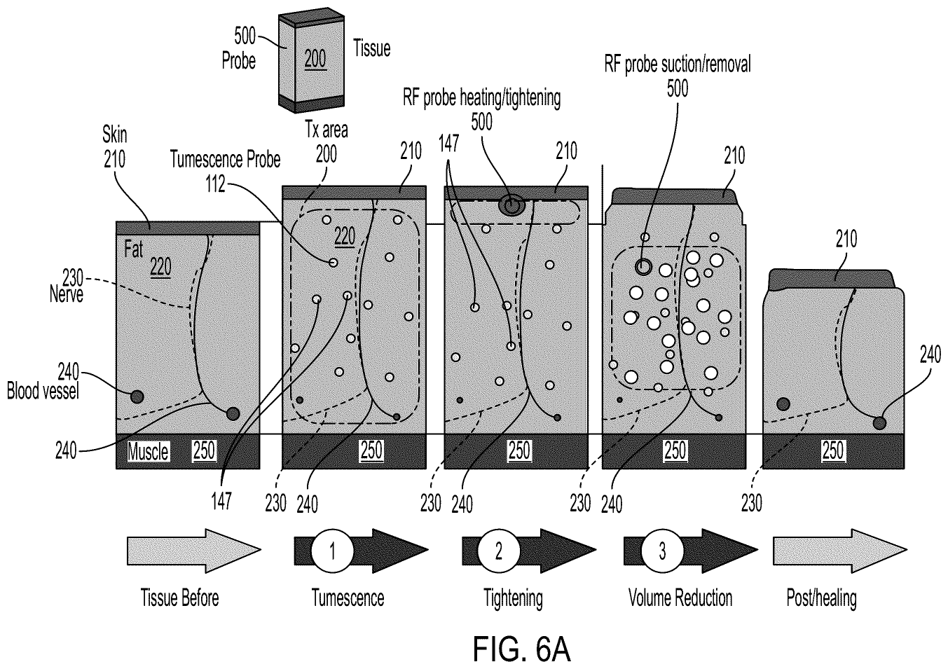

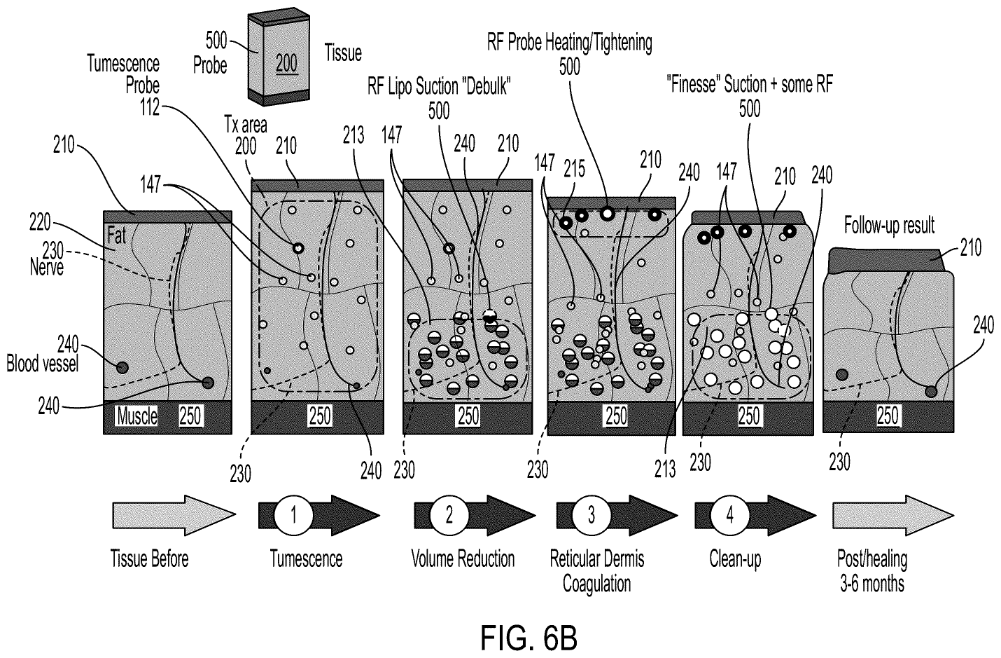

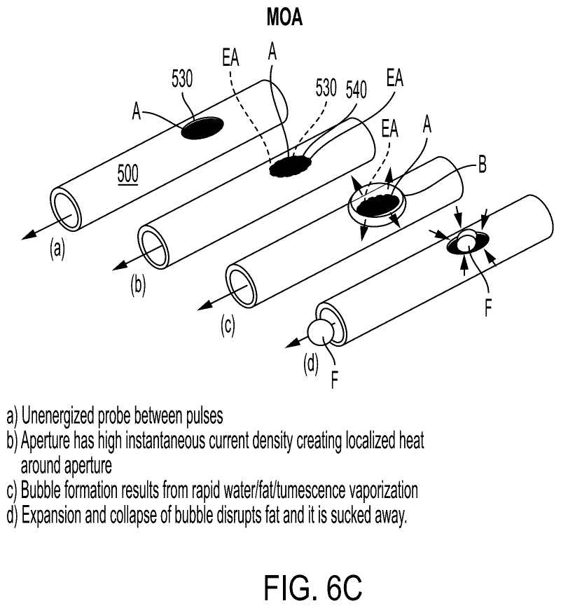

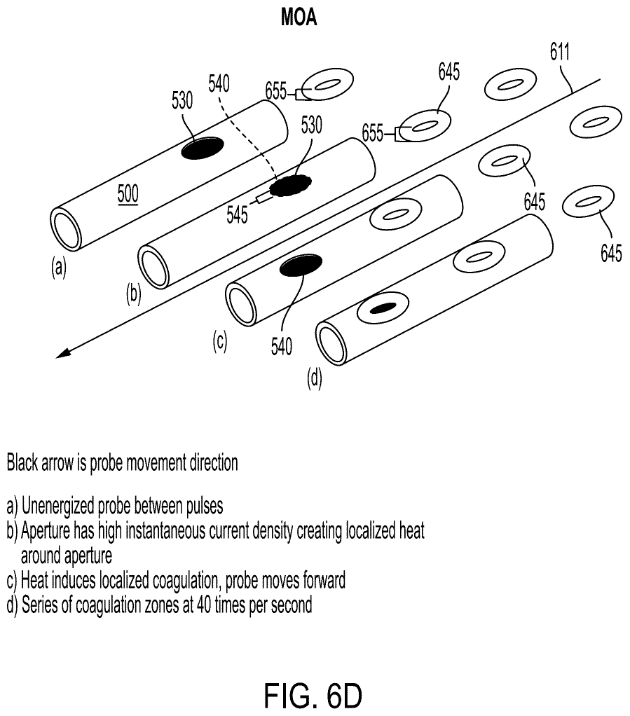

FIG. 6A depicts the expected method of tissue/suction removal using an exemplary RFAL probe in continuous RF power mode. However, as discussed above, RF assisted liposuction in continuous mode was found to cause thermally unconfined heating that can result charring of tissue on the cannula and/or a clog of the cannula that can render the probe unusable requiring disposal of the probe. Referring again to FIG. 6A and also to FIG. 1B, “Tissue Before” is identified as a target for treatment and includes fat, blood vessels and nerves between the skin and the muscle tissue. In Step 1, tumescence is applied to the tissue, normally through a cannula the size and type of which is determined at the preference of the practitioner. Optionally, the cannula employed to apply tumescence is also the RFAL probe utilized later in the method with the addition of another port (e.g., an additional tumescence port). Alternatively, tumescence is applied to the body area using a separate small cannula that is wholly apart from the RFAL probe. Next, in Step 2, referring to FIGS. 1B and 2, using footswitch 563 to provide a relatively lower pulse of energy at a relatively higher repetition rate without suction, dermal collagen is heated to coagulation (resulting in tightening). The distal probe end is used for “tenting” the skin to ensure the heat is applied to the reticular dermis. The practitioner can complete Step 2 in either a constant fanning motion or by periodically stopping in an area to apply an injection of heat to a fixed area and then move on to the next area of treatment this approach treating a fraction of the reticular dermis in a manner similar to a “stamped mode.”

After heating the reticular dermis, in Step 3 the probe was then operated with the other footswitch 561 to channel the volume and attempt to remove fat from the treatment area. However, RF assisted liposuction in continuous mode was found to cause thermally unconfined heating with variable heat generation that is dependent upon the local tissue impedance of the adipose tissue. The result can be a clog of the cannula that terminates the ability for tissue to flow through the cannula for aspiration. Unlike cannula clogs that occur in PAL or other forms of liposuction, the clogs that occur during continuous mode RF can be extremely difficult or impossible to resolve due to charring of tissue on the cannula. As a result, it was determined that the aspiration portion of Step 3 should not be completed with continuous mode RF. Thus, with continuous mode RF step 3 should be skipped. Instead, with Steps 1 and 2 of FIG. 6A, continuous mode RF energy should be employed for tissue tightening via soft tissue coagulation to tighten relatively deep tissue (e.g., at a depth of a fiber septa network in a specific treatment region, which will vary based on the volume of unwanted adipose tissue content in a region to be treated) and/or more superficial tissue (e.g., at a depth of the tissue immediately below the dermis in a specific treatment region, which sometimes presents a challenge because the goal is to treat immediately below the dermis to even out the tissue appearance after treatment). The relatively deep tissue is at a depth that is deeper than the superficial tissue being treated according to the disclosure.

The RF probe 500 described herein can provide tissue tightening simultaneous with the fat removal step (e.g., during liposuction). The tissue tightening is via soft tissue coagulation and tightens relatively deep tissue (e.g., at a depth of a fiber septa network in a specific treatment region, which will vary based on the volume of unwanted adipose tissue content in a region to be treated) during the liposuction. Subsequent to aspiration of the unwanted fat via suction the RF probe may provide tightening to tissue that is more superficial, e.g., at a depth of the tissue immediately below the dermis in a specific treatment region, which sometimes presents a challenge because the goal is to treat immediately below the dermis to even out the tissue appearance after treatment. The relatively deep tissue is at a depth that is deeper than the superficial tissue being treated according to the disclosure.

The Steps 1 and 2 shown and described in FIG. 6A may be used alone or together with the methods described later in FIGS. 6B-6D.

### RF System Pulsed Mode

FIGS. 1B, 5F-5N and 6B are employed to discuss use of the disclosed system 100 of FIG. 1B in pulsed RF energy mode. While the probes 500 described in FIGS. 5F-5N are described in detail here for use in pulsed RF energy mode, the described probes 500 may also be employed in a continuous RF power mode or alternating between continuous RF power mode and pulsed RF power mode. Further, pulsed RF energy mode as described may be employed with all or a portion of any probe 500 disclosed and described herein. The system disclosed in FIG. 1B may be used in pulsed mode to achieve RF assisted liposuction, deep tissue tightening and superficial tissue tightening, while actively avoiding cannula clogging. Active avoidance of cannula clogging can be achieved using what can be referred to herein as finesse mode or clogging reduction mode. In pulsed mode the system delivers thermally confined heating to the tissue adjacent to the aperture of the RF annular electrode.

Referring now to FIGS. 5F-5I, the REAL probe 500 includes four separate apertures 530. Here there are four apertures 530 and each of the apertures is located about the perimeter of the probe 500 at a distance from the distal end of the probe tip 520. In one embodiment, the four apertures 530 include a first set of through hole apertures that are farthest from distal end of the probe tip 520 and a second set of through hole apertures, separate from the first set of through hole apertures, that are located closer to the distal end of the probe tip 520. Optionally, the two sets of through holes are in “cross configuration” where the second set of through hole apertures located about the perimeter of the probe 500 are at a location 90 degrees off set from the first set of through hole aperture such that about the perimeter of the probe 500 there are four apertures 530 each having centers that are approximately equidistant from the center of the next aperture about the perimeter of the probe 500.

Referring now to FIGS. 5F and 5G, in one embodiment, the set of aperture through holes 530 farthest from the distal end of the probe tip are at a distance of from 0 mm to 200 mm from the distal end of the probe tip 520 where the probe measures 250 mm and the set of aperture through holes closest to the distal end of the probe tip 520 are at a distance of from 0 mm to 200 mm from the distal end of the probe tip 520. A governing principle for aperture placement along the cannula of the probe is to ensure the probe retains structural integrity during use.

FIGS. 5M and 5N, depict a configuration similar to what is shown in FIGS. 5F and 5G. There are four aperture holes 530 total along the side walls that are produced in “cross configuration” with one set of through hole apertures (i.e., apertures one and two) that sit closer to the distal end of the cannula and a second set of through hole apertures (i.e., apertures three and four). Each aperture 530 shown has another aperture that opposes it on the other side of probe/cannula that are not shown. Apertures three and four are offset by 90 degrees about the circumference of the cannula from apertures one and two. FIGS. 5F and 5G have a solid end and do not have the end hole(s) 531 as are shown in FIGS. 5M and 5N. FIGS. 5M and 5N, show an exemplary probe tip 520 design an end hole is defined by a sidewall or cylindrical housing of the probe tip to eliminate dead space in the end of the probe. Referring now to FIG. 5M, the diameter of the end hole 531 can be the same or similar to the other apertures 530 on that probe and will likely be smaller than the diameter of the probe tip. Referring also to FIG. 5N, the diameter of the end hole 531 can be larger than the diameter of the other apertures 530 and closer to the diameter of the probe tip 520. The end hole 531 embodiments depicted in FIGS. 5M and 5N, should be regarded as a safety concern since it is cutting directly in front of the probe and presents some added risk of, for example, skin perforation.

FIG. 6B depicts the expected method of tissue/suction removal using an exemplary RFAL probe 500 in pulsed RF power mode. “Tissue Before” is identified as a target tissue 200 for treatment and includes unwanted fat 220, blood vessels 240 and nerves 230 between the skin 210 and the muscle tissue 250. In Step 1, tumescence 147 is applied to the treatment area tissue 200, normally through a tumescence probe 112 having a cannula the size and type of which is determined at the preference of the practitioner. Optionally, the cannula employed to apply tumescence is also the RFAL probe 500 utilized later in the method with the addition of another port (e.g., an additional tumescence port in probe 500, not shown). Alternatively, tumescence 147 is applied to the body area using a separate small cannula 112 that is wholly apart from the RFAL probe 500. Next, in Step 2, referring also to FIGS. 1B and 2, using footswitch 563 to provide pulsed RF power at a range from about 1 to 5 Joules per pulse or from about 2 to 3 Joules per pulse with suction to the treated region 213 resulting in volume reduction also referred to as RF Liposuction debulking. Next, in Step 3, dermal collagen is heated in region 215, in the absence of suction, with the RF probe to coagulation. The distal probe end is used for “tenting” the skin to ensure the heat is applied to the reticular dermis resulting in tightening. The practitioner can complete Step 3 in either a constant fanning motion or by periodically stopping in an area to apply an injection of heat to a fixed area and then move on to the next area of treatment this approach treating a fraction of the reticular dermis region 215 in a manner similar to a “stamped mode.”

A more detailed schematic diagram explanation of FIG. 6B Step 2 is described herein in association with FIG. 6C and more detailed schematic diagram explanation of FIG. 6B Step 3 is described herein in association with FIG. 6D.

Referring again to FIG. 6B together with FIGS. 1B and 5F-5N, in various embodiments of RF assisted liposuction, after most of the unwanted adipose tissue is removed from the treated region 213 in Step 2 an optional finesse or refinement stage may be included in Step 4. The finesse stage is labeled as “Clean-Up” in Step 4 and it is aimed at improving the appearance of the treated region 213 by removing additional unwanted adipose tissue while delivering decreased RF power that leads to very low, or negligible, overall tissue heating in the treated region 213 while decreasing the incidence of clogging of the probe 500 treatment cannula. During the optional finessing stage shown in Step 4, the delivered RF average power is substantially decreased, while short pulses of RF energy with high peak power are repeatedly delivered so that each finessing RF pulse leads to generation of steam bubbles in the vicinity of the one or more apertures 530 present on delivery cannula of the probe 500 when the conductive edge of one or more apertures 530 (e.g., exposed stainless steel edge 540 surrounding the aperture 530) are energized with RF energy. For example, the decreased RF average power can be set between 0.5 and 10 W, or more preferably 0.5 to 5 W, or most preferably to 0.5 to 2 W.

A treatment benefit of the finessing RF pulses is to enable the flow of suction, or aspiration, of unwanted adipose tissue flowing through the cannula of the probe by decreasing the incidence of clogging of the treatment cannula of the probe 500. In this way, interruption of unwanted adipose tissue removal is lessened and/or avoided due to the finessing RF pulses that avoid clogging by generating disruption in the vicinity of the one or more apertures 530 present on the cannula of the probe 500.

Generally, a relatively smaller diameter suction cannula would leave a smaller injury on the surface of the patient skin at the entrance point. Smaller skin injury is preferable in cosmetic liposuction procedures. The desire to use a smaller diameter cannula must be balanced with the observation that generally non-power-assisted treatment cannulas having a diameter of less than about 4 mm are associated with cannula clogging with fibrous tissue that can significantly decrease the rate of aspiration of unwanted adipose tissue being removed from the treatment region, [Young, V. Leroy, and Harold J. Brandon. “The physics of suction-assisted lipoplasty.” Aesthetic Surgery Journal 24, no. 3 (2004): 206-210.].

The finessing RF pulses deliver thermally confined heating of the tumescent aqueous media intermixed with adipose and/or fibrous tissue that is near the probe apertures 530 energized with pulsed RF power and generate steam bubbles. The expanding steam bubbles in the intermixed aqueous and adipose media will lead to generation and propagation of disruptive energy originating from the steam bubbles. The expanding steam bubbles in the intermixed aqueous and adipose media or the intermixed aqueous and fibrous media can generate and/or propagate pressure waves and/or shock waves originating from the steam bubbles. At least a part of the fibrous tissue that may be in the process of clogging the cannula of the treatment probe 500 is likely to near the probe 500 aperture(s) 530 energized with pulsed RF energy. The combined effects of steam bubble formation, expansion, contraction, and a pushing force, possibly pressure waves and/or possibly shock waves will lead to disruption of the fibrous tissue and likely prevent and/or decrease the incidence of clogging in the cannula of the probe 500. For example, the finessing RF pulses are delivered in an energy control mode, having an energy range of from about 0.5 to 3 about J/pulse, with high peak power, larger than about 1 kW, delivered for short RF on-times, for example, on-times ranging between about 1 ms and about 10 ms as determined in real-time by the energy control system.

Referring still to FIG. 6B, the continuous suction applied to the cannula in Step 4 will bring a continuous stream of unwanted adipose tissue and some fibrous tissue to be removed through the probe 500 apertures 530 energized with pulsed RF energy (i.e., annular electrodes disposed on the cannula of the probe).

Depending on the suction pressure differential and cannula geometry together with the local treated tissue properties, the finessing energized RF pulses must be delivered at a sufficiently high repetition rate to disrupt fibrous tissue clogging while keeping the repetition rate sufficiently low to avoid unwanted tissue heating. For example, the repetition rate of the RF pulses during the finessing stage can be set between 0.5 and 5 Hz, or more preferably 0.5 to 2 Hz.

Another example of the application of thermally confined RF pulses aimed at preventing or decreasing clogging is in liposuction cannulas used to remove the bulk of the unwanted adipose tissue. The thermally confined RF pulsing can be implemented in addition to or instead of the cannula venting and tip and port(s) geometry designs that have been used to increase aspiration rates and to decrease the incidence of clogging [Young 2004]. The treatment benefit of finessing RF pulses is that the sporadic intentional disruption of all or a portion of a blockage in the one or more apertures disposed in a cannula employed for liposuction by a pushing force originating from steam bubbles keeps the flow of suction and/or aspiration of unwanted adipose tissue.

For example, in an exemplary pulsed RF power liposuction step (e.g., FIG. 6B step 2) the energy applied can range from about 1 Joule per pulse to about 5 Joules per pulse delivered at a pulse frequency that ranges from about 5 Hz to about 100 Hz. In an exemplary pulsed RF power finessing or clean up step (e.g., FIG. 6B step 4) the energy applied can range from about 1 Joule per pulse to about 5 Joules per pulse delivered at a pulse frequency that ranges from about 0.5 Hz to about 10 Hz.

Employment of finessing RF pulses to decrease the incidence of clogging of the treatment cannula can be extended beyond RF assisted liposuction to traditional or state of the art methods of liposuction. For example, referring to FIG. 5O, a clog avoidance element or finessing element 580 can be added to a state-of-the-art liposuction system, e.g., a PAL system. Likewise, the tissue disruptor 580 may be employed to clear unwanted tissue from an aperture of any lumen or cannula employed to remove tissue from a treatment area not limited to unwanted adipose tissue. In one embodiment, a finessing element or tissue disruptor 580 is employed to generate steam bubbles short electrical pulses to the one or more apertures (e.g., annular ports) on the state-of-the-art liposuction cannula (e.g., the PAL cannula). In one embodiment, disruptor 580 is a resistor 584, for example, a thin film resistor heater that facilitates thermally confined heating of the aqueous media surrounding the resistor. The thin film resistor may take the shape of metalized region(s) near the suction port(s) or aperture(s) on a cannula made of material with low electrical conductivity, for example PEEK or Delrin.

In some embodiments a tissue disruptor 580 may be used to retrofit an existing liposuction system and can be slidably disposed or received by or with respect to a legacy cosmetic probe. In some embodiments, the tissue disruptor may include a hollow cylindrical structure having a wall thickness and sized to compliment a shape and one or more dimensions of one of the outer diameter or the inner diameter of a cannula employed to suction tissue; at least one disruptor aperture disposed through the wall thickness, the disruptor aperture sized to compliment dimensions of a cannula aperture disposed through a cannula wall thickness in a cannula employed to suction tissue and the disruptor aperture is positioned to align with a cannula aperture; a resistor is disposed about an edge of the disruptor aperture, the resistor positioned to generate heat sufficient to thermally confine heating of an aqueous media surrounding the resistor.

Alternatively, the finessing element or disruptor 580 is a thin film resistor that is electrically insulated from a cannula made of electrically conducting material, for example stainless steel. The thin film resistor 584 can be shaped as an incomplete circle having a gap 586 at or near the edge of the suction port or aperture 582 of the disruptor 580. For example, disruptor 580 includes a resistor region 584 shown as a black incomplete circle in FIG. 5O. The width of the resistive region 584 may be chosen to be sufficiently narrow for efficient heat confinement in the surrounding heated aqueous medium and sufficiently wide to deliver necessary or sufficient electrical power to the one or more apertures disposed in a cannula employed for PAL liposuction without damage to the resistor. The material for the thin film resistor can be chosen to provide the resistance necessary to deliver between about 0.1 Joules and about 5 Joules of heating energy per aperture during electrical on-time that ranges between 1 ms and 10 ms. For example, the thin film resistor material can be chosen from platinum, tungsten and various alloys including Nickel-chromium or Iron-chromium alloys. The two ends of each resistor are connected to a power supply that can be electrically isolated for improved patient safety. In some embodiments, the heat confinement described herein also corresponds to localized heating in some embodiments. In some embodiments, heat confinement is promoted relative to the aqueous medium surrounding the cannula. In some embodiments, heat confinement is in the aqueous medium surrounding the cannula. In some embodiments, heat confinement is relative to the aqueous medium surrounding the cannula. In some embodiments, heat confinement is initiated, promoted, generated, or otherwise achieved in response to the selection of one or more parameters disclosed herein such as probe parameters of an RF signal.

Optionally, referring still to FIG. 5O, the finessing element or disruptor 580 can be an aftermarket add on that can be suited to in size and shape to existing PAL system cannulas such that the one or more aperture in the clog avoidance element or the finessing element will appropriately align with one or more apertures in a given model of cannula. In one embodiment, the inner dimensions of the aftermarket disruptor 580 will be suited to compliment the outer dimensions of the standard PAL liposuction cannula and its aperture(s) 582, resistor(s) 584 and optional resistor gaps 586 will likewise suit the PAL liposuction cannula such that these line up adjacent to the desired portions of the given model of cannula (e.g., cannula aperture(s)). Alternatively, in another embodiment, the outer dimensions of the aftermarket disruptor 580 will be suited to compliment the inner dimensions of the standard PAL liposuction cannula and its aperture(s) 582, resistor(s) 584 and optional resistor gaps 586 will likewise suit the PAL liposuction cannula.

In some embodiments, the tissue disruptor may include a tissue disruptor and cannula assembly, comprising: a cannula employed to suction tissue, at least one cannula aperture defined by a cannula wall, the cannula wall having a cannula wall thickness; a disruptor comprising a hollow cylindrical structure comprising a disruptor wall having a disruptor wall thickness and inner dimensions sized to compliment the outer shape and dimensions of the cannula; at least one disruptor aperture defined by the disruptor wall, the disruptor aperture is sized to compliment all or a portion of the cannula aperture dimensions, the disruptor aperture having an edge defined by the disruptor wall; a resistor disposed about the edge of the disruptor aperture, the resistor configured to generate sufficient heat sufficient to thermally heat a volume of an aqueous media surrounding the resistor, wherein the hollow cylindrical structure of the disruptor is sized and arranged to align the disruptor aperture and the cannula aperture, wherein the disruptor aperture aligned with the cannular aperture define a channel through both the cannula wall and the disruptor wall.

Referring still to FIG. 5O, a clog avoidance element or tissue disruptor 580 for use with a state-of-the-art liposuction system, may be employed to clear unwanted tissue from an aperture of any lumen or cannula employed to remove tissue from a treatment area (e.g., not limited to a treatment area having unwanted adipose tissue). The disruptor 580 is employed to generate steam bubbles to the one or more apertures (e.g., annular ports) of the liposuction cannula (e.g., the PAL cannula) using an energy source other than or in addition to an RF energy signal. For example, the disruptor 580 may employ, for example, a piezoelectric transducer, ultrasound energy, and/or microwave energy to clear unwanted tissue from an aperture of the cannula to avoid and/or remove clogs in the cannula.

In one embodiment, referring to FIG. 6B, a practitioner treats a region of patient tissue with pulsed RF liposuction (step 2), heating and tightening (step 3), and clean up/finesse (step 4). Referring now to FIGS. 5F, 5G and 5J, using footswitch 561 a relatively higher pulse of energy is provided to disrupt fat near the four apertures 530 on the probe 500 while suction is applied (resulting in liposuction).

The probe 500 cannula has an inner diameter measuring in the range of from about 2.7 to about 2.9 mm, which is comparable to a 4 mm outer diameter liposuction cannula used in standard PAL applications. Referring now to Step 1 of FIG. 5J, the pulsed RF energy causes high current density and creates localized heat about the exposed conductive material (such as stainless steel) non-insulated conductive edge 540 of each of the four apertures 530. As a result of the localized heat, bubbles form in the tumesced adipose tissue in the region of treatment. Thus, there is a formation and collapse of bubbles in the tumesced adipose tissue in the region of the apertures 530 as the practitioner moves the cannula linearly in a treatment direction 611 in the body area being treated. The expansion and collapse of the bubbles disrupts the adipose tissue enabling it to be pulled into the cannula. The disrupted adipose tissue improves the ability of the unwanted fat tissue to be suctioned without sticking to help keep the flow of the heated fat tissue 565 so that the lysed tissue 565 can be removed from the treatment region via the probe 500 cannula.

Next, Step 2 of FIG. 5J can occur subsequent to liposuction described in Step 1. Using footswitch 563 a relatively lower pulse of energy is provided at a relatively higher repetition rate without suction to heat dermal collagen to coagulation (resulting in tightening). During Step 2, in some embodiments, heating via pulse RF without suction, a suitable pulsed RF power applied can range from about 0.5 Joule per pulse to about 3 Joules per pulse delivered at a pulse frequency that ranges from about 0.5 Hz to about 100 Hz. Here the practitioner creates thermally confined heated zones near the reticular dermis to coagulate regions 645 of adjacent dermal tissue. In various embodiments, regions 645 may also be volumes or injuries or lesions. This can be accomplished by constantly fanning the probe and/or by periodically slowing the rate of probe movement and/or stopping moving the probe in a tissue area to treat the reticular dermis with the desired level of heat coagulation. The pulse frequency and cannula movement rate determine the density of the lesions 645 in a given area of dermal tissue. Depending on the rate of movement, the coagulation injuries 645 can overlap or nearly overlap and appear as stripes of coagulated tissue 645 (relatively slow movement rate) or the coagulation injuries 645 can be multiple separate toroidal like or substantially toroidal regions of coagulated tissue 645 (due to a relatively fast movement rate and as shown in FIG. 6D) at the dermis/fat junction.

Next, Step 3 of FIG. 5J can occur after the coagulation and/or tightening described in Step 2 and is used to clean up and refine the liposuction from Step 1 of FIG. 5J. Using footswitch 561 a relatively higher pulse of energy is provided at a relatively lower pulsed RF rate delivering decreased RF power that leads to very low overall tissue heating in the treated region while decreasing the incidence of clogging of the treatment cannula to disrupt fat near the four apertures 530 on the probe 500 while suction is applied (resulting in clog disruption and/or clog avoidance). The delivered RF average power is substantially decreased, while short pulses of RF energy with high peak power are repeatedly delivered so that each finessing RF pulse leads to generation of steam bubbles in the vicinity of the one or more apertures 530 present on delivery cannula of the probe 500 when the one or more apertures 530 are energized with RF energy about the exposed conductive edge of the aperture 540. The treatment benefit of finessing RF pulses is that it provides a sporadic intentional disruption of all or a portion of a blockage in the one or more apertures 530 disposed in a probe 500 cannula due to a pushing force caused by steam bubbles and this keeps the flow of suction and/or aspiration of unwanted lysed adipose tissue 565, avoiding clogging and/or downtime thereby reducing and/or avoiding downtime. In an exemplary pulsed RF power finessing or clean up step the energy applied can range from about 1 Joule per pulse to about 5 Joules per pulse delivered at a pulse frequency that ranges from about 0.5 Hz to about 10 Hz. In contrast, in an exemplary pulsed RF power liposuction step (e.g., FIG. 6B step 2) the energy applied can range from about 1 Joule per pulse to about 5 Joules per pulse delivered at a pulse frequency that ranges from about 5 Hz to about 100 Hz.

The cannula of the probe 500, for example, the cannula shown in FIGS. 5F and 5G, may be made from various conductive materials such as stainless steel. In one embodiment, the cannula is spray coated with PVDF to provide insulation that avoids electrical leakage where it has been applied. However, there is concern that PVDF degrading around the aperture holes may leave some PVDF behind in the body of a treated patient. In another embodiment, the cannula, for example the cannula shown in FIGS. 5F and 5G, is made from stainless steel and the exterior of which is insulated with FEP polymer. FEP has a relatively high melt/degradation temperature relative to PVDF. In one embodiment, the FEP is in the form of a shrink-wrapped material (e.g., shrink wrapped tube) that is attached to a stainless-steel cannula via heat and has a thickness of approximately 8/1000ths about the length of the cannula and the thickness of the FEP may be a little thicker at the tip of the cannula. FEP lacks an electrical property differential at 500 kHz versus 4 MHz. The FEP shrink wrap application can be tested by verifying the absence of electrical leakage from the probe 500 of the cannula.

Where the cannula of the probe 500 has the four apertures 530 (e.g., a first set of through holes in cross configuration with a second set of through holes) as is shown in FIGS. 5F and 5G the insulative layer or coating 550 (e.g., FEP shrink wrap) is pulled over the stainless steel cannula and the portions of the FEP shrink wrap 550 that are adjacent to the aperture(s) 530 are removed to leave a void in the aperture location (e.g., punched through via a biopsy punch). Thereafter FEP shrink wrap is heated to attach the FEP to the stainless-steel cannula. In some embodiments, when RF energy is applied to the cannula, the FEP covers the top surface of each of the apertures such that RF comes out from under the surface that is shrink wrapped through the exposed stainless-steel portions 540 of the aperture made up of the thickness of the cannula. In other embodiments, the insulative coating 550 is removed from the void of the aperture 530 such that at least a portion of the cannula material (e.g., stainless steel) surrounding the aperture void is also exposed, for example, a biopsy punch punches holes in the insulative coating material that are slightly larger than the apertures in an embodiment of a shrink wrapped tube the heated shrink wrapped insulative coating leaves some portion of the aperture along the length of the cannula as exposed stainless-steel 540.

Where the cannula 525 has four apertures and has a rounded distal end as shown in FIGS. 5F and 5G, during treatment the collagen scaffolding of the target area of tissue may be preserved, and the treatment of the patient's tissue can be completed with finesse to maintain/preserve structures in the body in the region of the target tissue. Where the cannula has four apertures 530 and the probe tip 520 has an end hole 531, as shown in FIGS. 5M and 5N, the treatment may be more aggressive and can open the risk of tissue perforation. The pulsed RF treatment with four apertures 530 described with reference to FIGS. 5F and 5G combine to balance the need for consistent suction and avoidance of downtime while lessening the risk of scarring due to over treatment and or puncture risk due, for example, to an end hole 531 shown in FIGS. 5M and 5N.

Probes suitable for use with RFAL and/or tightening may be disposable, reusable, partially disposable, or partially reusable. In one embodiment, a timer (e.g., a chip) is employed to prevent reuse based, for example, on a time period of probe use that ranges from about 1 hour to about 12 hours, from about 2 hours to about 10 hours, and from about 4 hours to about 6 hours. In one embodiment, the cable is reusable that attaches the probe handle to the system console (e.g., the RF system console). In another embodiment, both the cable and the handle are reusable. The probe cannula may be disposable and can be inserted into the handle end, twisted, and locked in place for use. Alternatively, the probe cannula can be cleaned and reused one or more times between uses. In one embodiment, the temperature is sensed such that RF delivery is controlled based upon temperature resistance whereby impedance is sensed to determine temperature. Voltage can be adjusted based on impedance or RF delivery may be adjusted based on impedance. The system can read the overall resistance. The position of the return electrode relative to the site of entry into the patient. The resistance may also be informed by (a) the thickness of the patient (b) the body composition (e.g., connective tissue and visceral fat) and (c) the fluid being administered. The voltage may be adjusted based upon the detected impedance. The length of each pulse can be managed such that the time between each pulse and/or the peak power of each pulse can be adjusted based upon what impedance is detected.

Referring again to FIGS. 5F-5I, handle 510 is connected to the probe cannula 525 and all or a portion of the probe cannula 525 can be thermally insulated. Various electrical insulators and thermal insulators can be applied to the cannula such as to its inner surface, outer surface, edges, and other regions. The tip 520 of the RFAL probe 500 is coated and the coating 550 has dielectric properties. Specifically, the coating 550 of the probe 500 can be made from or contain Fluorinated Ethylene Propylene (FEP) or be wrapped in FEP, for example, in FEP heat shrink. FEP is available under brand names including TEFLON. There are alternative electrical insulators that can be applied to the cannula. These alternative electrical insulators may include such as polyolefin, olefin, fluoropolymer (such as FEP, PTFE or Kynar), PVC, neoprene, silicone elastomer or Viton. Preferably, the dielectric material or coating on the body of the probe (e.g., on the tip 520 and/or on the shaft 525) has dielectric properties that are insulative for both the fat removal frequency (e.g., 200 kHz-10 MHz, or 350 kHz-7 MHz, or 500 kHz-4 MHz, or 500 kHz) and/or the heating frequency (e.g., 200 kHz-10 MHz, or 350 kHz-7 MHz, and 500 kHz-4 MHZ, or 500 kHz). The dielectric properties of the coating 550 has dielectric properties such that if active RF is applied to the stainless-steel cannula together with suction during the fat removal phase, RF energy is concentrated about the exposed non insulated portions of the perimeter of the one or more apertures 530 such that they act as suction ports during fat removal procedures. In some embodiments, the probe 500 can be sanitized and reused, optionally, the probe is designed to be used a limited number of times, for example, between 1 and 50 times. In other embodiments, the probe 500 is a single use disposable. A single use probe avoids risk of contamination from one procedure to another and avoids risks associated with wear to the coating, e.g., the PVDF coating during a lengthy procedure. Coating wear on the probe tip 520 during multiple uses or procedures having extended length or extended treatment duration may result in reduced RF and/or heating control during the heating and/or the tissue lysing treatment modalities described herein. In some embodiments, the single use disposable probe 500 may include materials and coatings that are durable such that they can complete continuous operation in tissue for times that range from about 10 to about 180 minutes, or from about 60 to about 120 minutes, from about 90 to about 100 minutes at, for example, 70% duty cycle or more, 80% duty cycle or more, or 90% duty cycle or more. In some embodiments the probe is employed to complete tissue lysing (e.g., liposuction) 70% of the time and minimally invasive tissue heating 30% of the treatment time, to complete tissue lysing (e.g., liposuction) 80% of the time and minimally invasive tissue heating 20% of the treatment time, or to complete tissue lysing (e.g., liposuction) 90% of the time and minimally invasive tissue heating 10% of the treatment time. In one embodiment, the tissue treatment is 120 minutes long with a tissue lysing (e.g., liposuction) performed for about 100 minutes and minimally invasive tissue heating is performed for about 20 minutes. In another embodiment tissue lysing (e.g., liposuction) is performed for about 75 minutes and minimally invasive tissue heating is performed for about 15 minutes.

Referring now to FIGS. 5A-5I, the probe shaft 525 and tip 520 measures in length from about 10 to about 50 cm, from about 15 to about 30 cm, at or at about 25 cm in length (this measuring the probe shaft 525 and tip 520 from where the shaft is adjacent the distal end of the handle 510). The probe tip 520 outside diameter (including the thickness of the coating 550 that insulates the probe wall) measures from about 1.5 mm to about 7 mm, from about 2 mm to about 6 mm, from about 2.5 mm to about 5.5 mm, from about 2.9 mm to about 5 mm, or from about 3.5 mm to about 4.5 mm.

Referring to FIGS. 5C, 5H and 5I, the probe 500 shaft 525 and/or tip 520 can feature a thermocouple 572, or multiple thermocouples 572 (i.e., redundant thermocouples) that make temperature measurements to provide treatment information. In some embodiments, multiple thermocouples 572 (e.g., two separate redundant thermocouples 572 that provide differing information due to their different locations along the length of the cannula of the probe 500) are disposed in the probe tip 520 and provide treatment information, for example, the temperature sensors (thermistors, thermocouples, etc.) are selected to have a rapid response time (less than 1 second) to accurately measure the temperature changes of the tissue that was induced by the application of RF energy. In some embodiments, as the RF energy is applied, the temperature of the tissue will be monitored and governed to a set point temperature that cannot be exceeded. In some embodiments, the probe 500 has no thumb vent port for vacuuming, but the probe 500 handle 510 couples with standard liposuction hose sets 567 at the lateral end. During an RFAL treatment at 500 kHz lysed tissue 565 exits the probe 500 via standard liposuction hose set 567 that is coupled to the lateral end of the probe handle 510. The handle 510 may be compatible with standard, low cost (not tapered) tubing or hose 567 sets for the suction connection back to a canister. In one embodiment, the probe 500 contains no RF or other controls. In other embodiments, RF controls are present on the probe 500. The probe 500 shown in FIGS. 5H and 5I may have a high peak power system (1.5 kW) and with a short pulse delivery (<7.5 ms). In average power clinical use of the probe ranges from 4 W to 60W where the average power is calculated as follows: the energy per pulse multiplied by the repetition rate set by the user.

The probe 500 depicted in FIG. 5A is designed to provide radially symmetric effects, specifically here, the radially symmetric effects are a consequence of having apertures 530 in the tip 520 that are three similarly sized elongated slots disposed in an equally spaced locus about the diameter of the tip 520. It is contemplated that a family of probes can be designed with some that include directional effects and others that include symmetric effects.

In another embodiment, referring to FIG. 5D, the probe 500 can be designed to create directional effects (ports to one side). Specifically, tip 520 has three apertures 530 that are all located at a single point of the diameter along the length of the tip 520 of probe 500. Directional aperture arrangements enable the surgeon to remove tissue only to one side of the probe, thus enabling more control (e.g., directional effect control).

Referring now to FIGS. 5C,5D and 5H, the handle 510 of the probe 500 can indicate to the user the rotational position of the handpiece. For example, the probe 500 can be faceted to have four quadrants 514. The probe 500 can have an indicator 512 that is visual and/or tactile to inform the user grasping the handle 510 of the location of aspects of the probe tip 520 during the invasive procedure when the tip is not visible to the practitioner. For example, in FIG. 5D, where a probe 500 has directional effect, the handle 510 has an indicator 512 where in one quadrant the handle 510 is indexed through tactile feel and/or a visual indicator to align with aperture(s) position on probe. This way, the practitioner who feels the tactile indicator 512 understands that the apertures 530 on the tip 520 are positioned along the longitudinal axis of the tactile indicator 512.

Referring still to FIGS. 5A-5E, there are many considerations that may be balanced to make the single probe 500 described herein function for RFAL and for skin tightening (e.g., minimally invasive skin tightening). The probe can be made from stainless steel tubing, which functions to provide an electrical conduit for the RF energy and to support the high axial loads as the tube is pushed through the treatment area with the full force of an arm through fibrous tissue. The diameter/length/wall thickness(es) all factor into a successful design which can only be verified during ex-vivo experiments or during the actual surgical procedure.

Some of the factors that go into a successful design of a liposuction probe are known and some need to be determined experimentally. For example, too much stainless-steel tube flexing removes control and too large a diameter limits the surgical viability. Smaller diameter probes can be used in shorter lengths to improve stiffness and control. Small diameter probes are desirable because they limit the entry incision size and enable treatment of smaller areas, but the internal lumen of the probe is selected to be large enough to allow lysed fat to suck freely through the lumen without clogging. The method of applying RF, continuously or pulsed, and aperture arrangement, the amount of power and current density about the probe's apertures all contribute to the propensity of a certain probe to clog. Maximizing the internal diameter will improve suction force and reduce the incidence of clogging. The location of the apertures along the axis of the probe can also be related to clogging incidence; apertures more distal are subject to less suction force and clog more frequently.

Referring now to FIGS. 5A-5I, the desired RF probe 500 shaft 525 or probe 500 cannula 525 outside diameter can range from about 1 mm to about 10 mm, from about 3 mm to about 5 mm. This size range of probe diameters are accepted as having the performance needed with probes having outside diameter measuring less than 6 mm having an incision site dimension that is currently typically used in standard SAL liposuction procedures. In some instances, the probe tip 520 has a smaller outside diameter than the shaft 525, here, the probe tip 520 has a diameter range of from about 0.5 mm to about 9.5 mm, from about 2 mm to about 4 mm. The inside lumen of the probe 500 cannula 525 is constant through the probe tip and the probe shaft with a diameter range of 0.4 mm to about 9.4 mm, or from about 1.5 mm to about 3.9 mm.

As described, the probe 500 coatings 550 may enhance the performance and behavior of the probe. Proper selection of biocompatible coatings may support toggling the probe between two different frequencies to achieve two different tissue effects by depositing the current in two different areas of the probe whether lysing fat or adipose tissue or heating reticular dermal tissue to achieve tightening via soft tissue coagulation (see, e.g., FIGS. 5E and 5J). Further, the selected probe coatings are chosen to be able to withstand many minutes (sometimes 90+) of constant use while not wearing out the coating or breaking down the coating risking changes to the desired tissue effect properties.

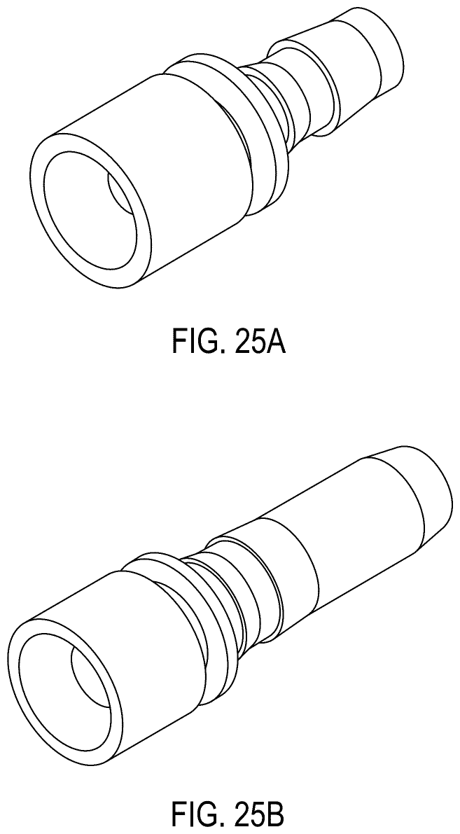

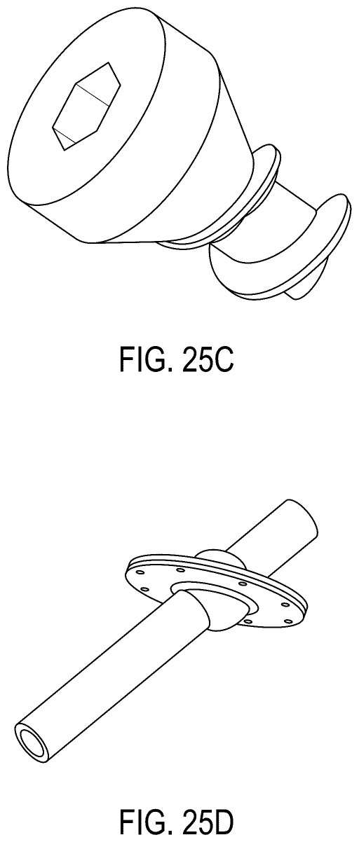

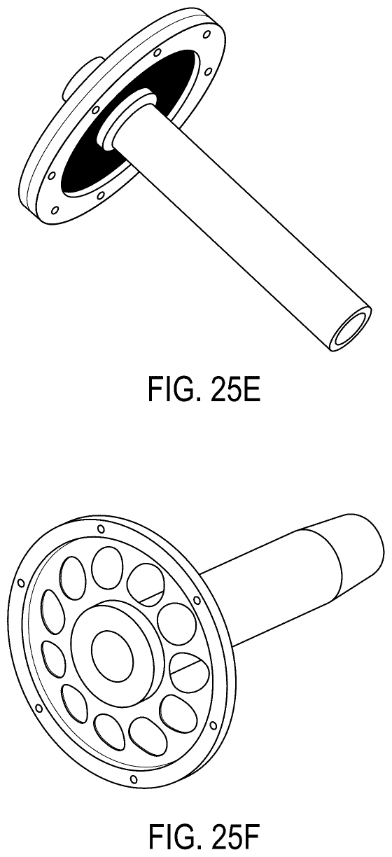

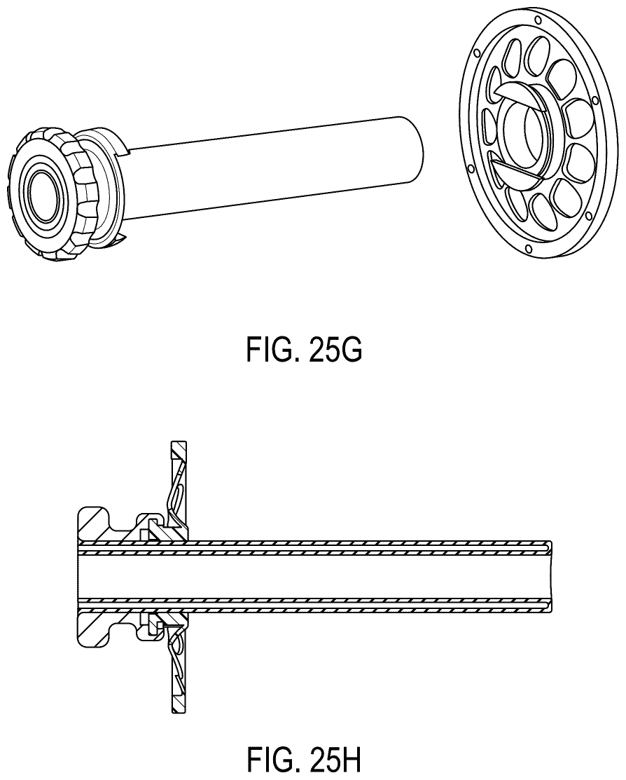

Referring now to FIGS. 5C, 5E, 5H, 5I, and 5J, the heated and/or lysed fat 565 will be brought to a temperature range that may extend from body temperature to the temperature of transient plasma formation, but settles to bulk temperatures from about 40° C. to about 70° C. and then will be aspirated down the lumen of the probe 500. The amount of power applied to the tissue and the means of pulsed or continuous RF delivery and closed-loop control mechanisms (temperature limits) will be major determinates towards the resultant heating of tissue. In some embodiments, the suction hose 567 is employed to aspirate the lysed fat tissue 565 from the aperture(s) 530 in the tip 520. When the heated fat tissue 565 (about 40-70° C.) is aspirated through the probe 500 lumen, the fat tissue 565 can possibly heat the probe shaft to temperatures that are too high for the skin (45° C. max) at the incision point. If no action is taken to control the probe shaft temperature, the skin at the incision point risks thermal injury. Thus, the probe shaft 525 may be thermally insulated from the surrounding tissue intended to be untreated, for example, the tissue at the incision point where the probe tip 520 of probe 500 is introduced into the body of the patient for the invasive procedure and this is discussed in further detail together with the description of FIGS. 25A-25H. The amount of RF power applied to tissue, whether it be pulsed or continuous and the temperature feedback from the thermocouple sensor(s) 572 which can be used to regulate the RF will together contribute to the temperature of the fat being aspirated through the probe 500 lumen, however, it is possible that some form of insulation of the probe shaft may be required.

Simple plastic insulation such an added layer of heat shrink may contribute to insulation of the probe 500 lumen. Such an insulation may be composed of an aerated plastic in a woven layer to introduce more air to help with thermal insulation. However, dimensional restrictions on the OD of probe tip 520 may limit the thickness of the insulation layer. A tube-in-tube probe lumen design that utilizes two stainless steel tubes with a thin air gap therebetween can provide the air gap providing insulation. The tube-in-tube approach with air gap therebetween can be employed to maximize thermal insulation with a small added thickness. A further improvement on the tube-in-tube design may also include evacuating the air therebetween, for example, by having a vacuum applied to the air gap instead. The vacuum approach may be effective and may reduce the required amount of thermal insulation but is relatively costly.

Referring now to FIGS. 5C-5E and 5H, 5I and 5J, the internal body tissue temperature is monitored by two temperature sensors 572 on the tip 520 probe 500 shaft. The location of the two probes 572 allows the user to measure local temperature close to the aspiration slot(s) 530 and temperatures farther away that better represent bulk tissue temperatures. Reporting the two temperatures in the treatment region adds a level of safety since they provide redundancy. The delta between the two temperatures can be relevant since temperature sensor 572 close to the distal end of the tip 520 may be used to regulate the RF and maintain the set temperature in heating mode. This temperature delta measured by the two probes 572 allows a user to monitor the tissue heating endpoint and resulting bulk temperature. The two temperature probes 572 will also monitor the heat/temperature of the fat in the lumen and/or the temperature of the body area being treated during fat removal mode. The choice of temperature sensor(s) can be varied, but use of a small mass of thermocouples allows for smooth probe shafts without “bumps”, however, small protrusions created by thermocouples are tolerable. As shown in FIGS. 5H and 5I, the one or more thermocouples 572 may be covered by one or more layers of insulation, for example, by three separate layers of FEP.

In some embodiments, referring now to FIG. 5D, a collar 555, e.g., a stainless-steel collar, is added to the tip 520 near the distal end of the probe 500. The purpose of this collar 555 is to protect the leading edge of the heat shrink from the forces applied as the probe is thrust into fibrous tissue. Without such protection, the heat shrink insulative coating may flare over time and peel back. Thus, the collar 555 can prolong the life of the probe by avoiding degradation of the probe and/or the probe insulation.

For a probe construction which only uses FEP coating throughout the length of the probe and does not use the combination of FEP and PVDF, the collar 555 may not be required.

Preferably, the handle 510 is lightweight. Because RFAL treatment offers an case of pushing through tissue, the handle and probe have a comfortable feel or an attractive feel. In one embodiment, referring to FIGS. 5C and 5H, the probe has directional ports and the handle is designed to have a tactile feature that aligns with the aspiration port direction, so that the user grasping the handle 510 will have an indication of the aspiration portion location/orientation along the length of the probe 500 even when the probe is submerged into the tissue. In the described embodiments, RF control is left off of the handle as well as a suction vent since the handle may be held in each quadrant of rotation. In one embodiment, a single wire and suction hose connect the probe device to the console. Further embodiments might include one or more of RF controls or a vacuum thumb hole vent as additional or alternative control features on the probe or on the console.

Referring now to FIGS. 1B, 5C, and 5H a cable 571 that attaches the handpiece 510 to the console and will have all the necessary conductors to transmit RF to the handle 510 of the probe 500 and the requirements to simultaneously measure the temperatures of the RF probe at the thermocouples 572 with the RF activated back to the console. A one-wire encrypted ID device can identify the probe 500 to the console and determine the probe lifetime, for example, the one-wire encrypted ID device may be in the connector between the probe and the console.

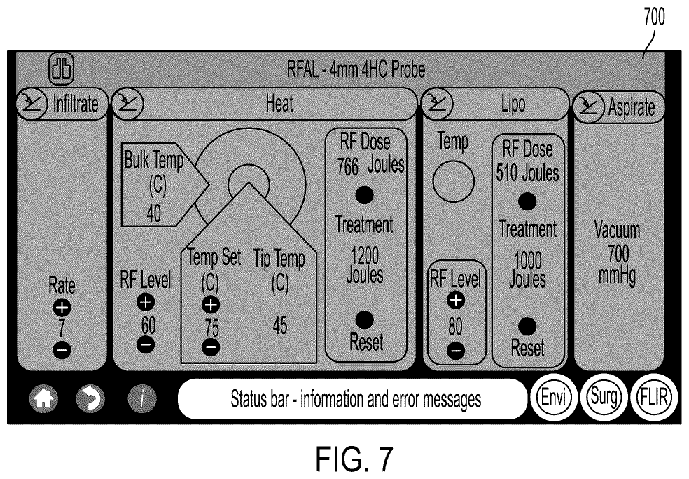

An example of a graphical user interface (GUI) 700 disposed on a console of a treatment system, such the system of FIG. 1B, is shown in FIG. 7. The GUI 700 shows each of the operating modes: Infiltration, Heating, Liposuction, and Aspiration. Each of the operating modes, namely, Infiltration, Heating, Liposuction, and Aspiration, can be selected independently, however, Liposuction (or RFAL mode) can combine with the use of aspiration, for example, by being used in tandem.

The clinical goals of the System of FIG. 1B is to generate one probe that can be used for heating tissue and without removing the probe to switch to fast resection. The RFAL approach is expected to provide superior speed of fat removal that will reduce fatigue, allow for adjustable control of tissue removal rate, speed up operations and reduce the frequency or number of strokes of the reciprocating action that is required of the practitioner. The REAL approach will enable preservation of the structural integrity of the septae and enable tissue discrimination that preserves necessary connective tissue.

The construction of the probe tip is configured to support easy passage of the probe tip through tissue. A plastic tip will not have the electrical properties or thermal mass required to (1) heat up tissue via RF and/or (2) serve as a heated leading edge to case passage through tissue. Thus, in some embodiments, the probe tip is constructed of stainless steel. Alternatively, the probe tip may be made from another biocompatible metal material. In order for the probe tip to be able to perform heating and/or liposuction treatment, the metal material may be coated with one or more insulative materials. Coatings suitable for application to the exterior of the probe tip are polytetrafluoroethylene (PTFE) or fluorinated ethylene propylene (FEP), which are sold under the trade name Teflon. These have a much higher operating temperature before melting as compared to PVDF. FEP is more easily formable than PTFE and has advantages in forming the coating over the end of the probe in embodiments when it the probe end has a domed shape. The electrical properties of PTFE and/or FEP do not favor the dramatic difference in dielectric properties that differentiate 500 kHz and 4 MHz like PVDF, but both PTFE and/or FEP can work to the clinical endpoint(s) envisioned and described herein.

The PTFE may be used for the 500 kHz liposuction—as well or better than PVDF. Since PTFE is more commonly manufactured, lead times and costs associated with PTFE are reduced relative to PVDF. While PTFE may work for tissue heating at 4 MHZ, it does not exploit the ability of the coating to leak through at the higher frequency as is present when PVDF is employed, but rather, the current at 4 MHz will be concentrated about the apertures of the cannula similar to the concentration during liposuction. PTFE does have a higher melting temperature than the PVDF probe tip with either PTFE or PVDF was previously discussed herein, however, the inside of the cannula wall may in some embodiments also benefit from a lubricious coating such as PTFE (e.g., Teflon) to aid the flow of the lysed fat aspirate as it flows down the tube to be removed from the subject's body.

In one embodiment, all or a portion of the interior of the cannula is electrically insulated. Insulating the interior of the cannula in addition to having an insulated exterior would provide a very limited conductive region. For example, the exposed edge of the cannula wall comprising conductive material is the only portion of cannula that acts as a conduit of conduction of RF energy. In this way, the energy density near the edge of the aperture is highly concentrated. Further, insulating in addition to the exterior, the interior of the cannula could offer benefits including, for example, maintaining a more consistent impedance during usage. Where insulation is present on both the exterior and interior of the cannula the effective load impedance would increase relative to a cannula that has insulation only on the exterior and as a result control would need to be adjusted accordingly. Finally, the inside of the cannula wall may in some embodiments also benefit from a lubricious coating such as PTFE (e.g., Teflon) to aid the flow of the lysed fat aspirate as it flows down the tube to be removed from the subject's body.

In one manifestation, FEP can be used to electrically and thermally insulate the cannula length but leaving a portion of the probe tip uninsulated. A second complete FEP layer will double the insulating along the cannula and cover the tip. In this way, the FEP thickness will be half (or any ratio of the two layers thicknesses) at the tip and promote more current leakage on the coated area of the tip and act more like the PVDF, frequency dependent design solution.

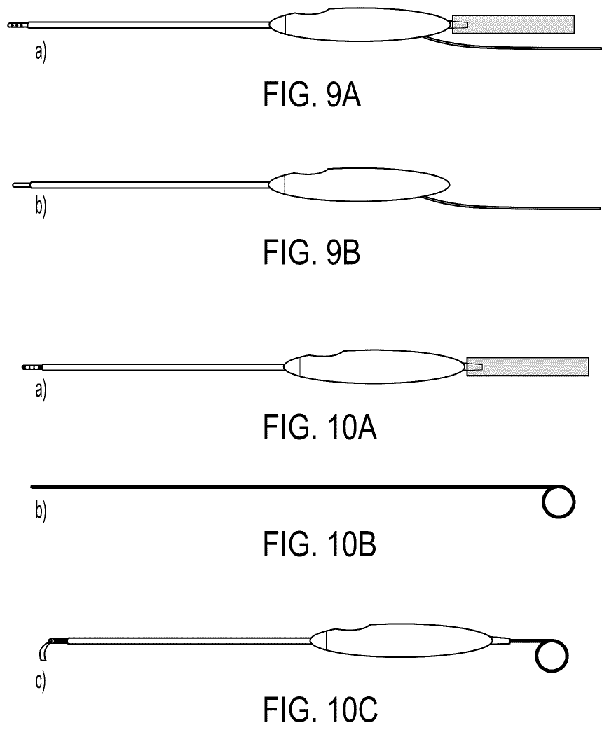

In some embodiments, two probes may be used to implement one or more of the methods and tissue treatments disclosed herein, referring to FIGS. 9A and 9B one is the disposable liposuction probe as described in this disclosure and is shown in FIG. 9A. The other, referring to FIG. 9B, is a separate RF heating only probe which can also be disposable, but to keep procedure costs down may be reusable and autoclavable. The separate RF heating probe shown in FIG. 9B may have a relatively small diameter (e.g., from about 2 to about 3 mm), no internal lumen, and with an uncoated stainless-steel electrode for heating. The separate RF heating probe shown in FIG. 9B would feature temperature monitoring via one or more thermocouples placed on the RF heating probe.

Since the RF probe shown in FIG. 9B is adapted for both heating skin tissue (e.g., heating reticular dermal tissue) and for lysing for liposuction, during a phase of heating reticular dermal tissue the RF probe will be used for heating without tissue suction. At least some tissue can enter the cannula Inner Diameter (ID) during the phase of heating reticular dermal tissue to achieve tightening via soft tissue coagulation. This tissue may eventually build up in the ID and become clogged into the ID since no suction will be applied in the phase of heating reticular dermal tissue for tightening. To avoid the potential downtime and other challenges associated with such clogging, it is possible to fill the RF probe ID with a rod-like stylet that plugs into the back of the probe and fills the entire ID of the cannula during heating mode. This stylet can also be used to clean clogged tissue from the probe.

FIGS. 10A-10C depict the probe assembly with a suction tube attached. If it is desired to fill the cannula ID in order to prevent tissue from building up during modes which do not require suction, the suction tube can be removed and a stylet FIG. 10B whose OD matches the ID of the cannula can be inserted into what is shown in FIG. 10A thereby filling the ID volume with the stylet of FIG. 10B to provide the assembly as is shown in FIG. 10C.

Referring now to FIGS. 5K and 5L, provided are two images of a probe tip 520 having six apertures 530 made by three through holes (through holes are symmetrical and go through the probe tip from one side along the length of the probe 500 to the symmetrically opposite side). There are advantages to this cross-hole geometry pattern, specifically, the cross holes are easy to clean. The determination of how many holes, for example, from 2 holes (1 set of cross holes) to 6 holes (3 sets of cross holes), and the size of each aperture is a sum of the probe impedance and the tissue impedance as well as the system capabilities for delivering power into the impedance determined by the number and size of the apertures and the patient's bulk body impedance. For a given cannula having a selected diameter and number of apertures, the number of Joules per pulse delivered at from about 5 Hz to about 100 Hz, or about 20 Hz may be selected to not clog and achieve acceptable resection rates and suitable or targeted tissue heating. FIG. 5L shows a longitudinal axis of the probe/cannula. Each of the apertures 530 has a perimeter, such as a circular or an elliptical perimeter. In some embodiments, two apertures are arranged relative to each other on opposing sides of the probe such that a line L perpendicular to the longitudinal axis LA is co-linear with a point disposed in perimeter of a first aperture and another point disposed in the perimeter of an opposing second aperture. Two such lines L are shown for two different opposing aperture pairs.

### Using the Acoustic Feedback of Pulsed Mode to Determine Energy Setting

In some embodiments when using a pulsed RF energy mode, an acoustic popping sound that is emitted may be used to determine if something useful is happening to tissue being targeted for treatment. The method of determining this threshold is to begin at a low setting and increase the number of Joules until a reliable audible or tactile signal is achieved. To be conservative, a user may increase the energy 10-20% above this threshold and continue to monitor popping noises and tissue vibration with the non-dominant hand.

The probe and handle assembly disclosed herein may be disposable after a single use. Alternatively, they may be separable parts where portions are reusable and other portions are disposable.

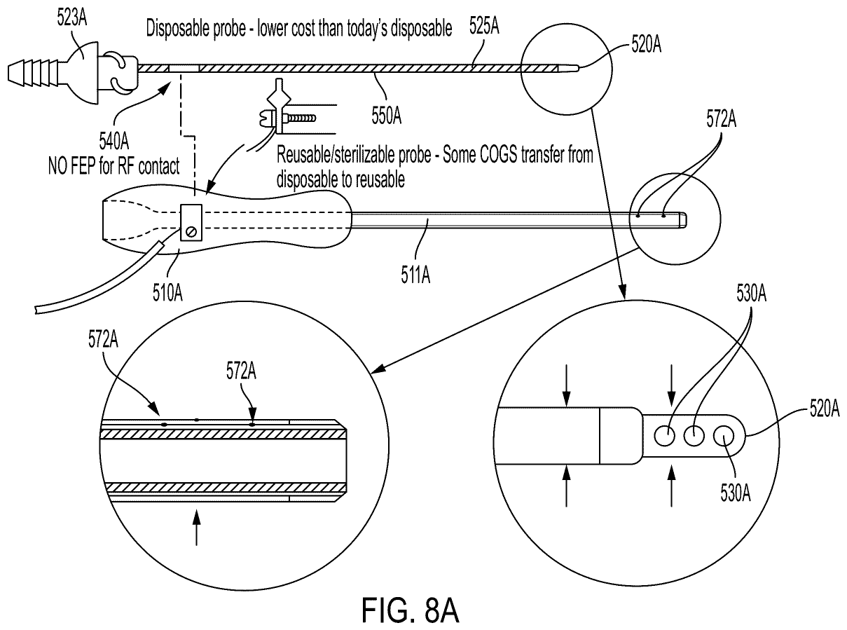

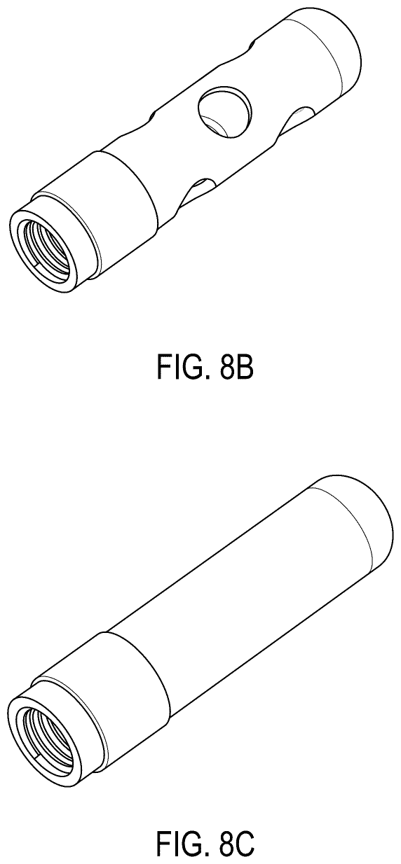

Referring now to FIG. 8A, the reusable handle 510A, shaft cover 511A and temperature monitoring portions 572A (e.g., thermocouples 572A) of the handpiece may be reusable, e.g., autoclavable/sterilizable for a number of times, e.g., autoclavable from 1 time to 20 times or autoclavable 10 times. A disposable cannula 525A (i.e., shaft 525A) may be partially insulated 550A and also include portions of exposed conductive material 540A (e.g., stainless steel) and the disposable cannula 525A will be used in concert with the reusable handle 510A and temperature monitoring portions 572A of this alternative assembly. Specifically, the disposable probe tip 520A having one or more apertures 530A is inserted into one end of the reusable handle 510A and the disposable probe cannula 525A fits within the inner diameter of the handle 510A and reusable handle shaft cover 511A and is removable attached to the handle 510A via the disposable probe connector 523A. Note, the apertures 530A of FIG. 8A can be of the size, type, and configuration described herein as aperture 530. Here, the reusability of the handle 510A, cannula cover 511A and temperature monitoring portions 572A of the handpiece will add to the outside diameter of the final design but offers another small insulating layer between the assemblies.

In another embodiment (not shown), at least a portion of the probe cannula is also reusable and autoclavable with the remainder of the reusable handle and thermocouple portions of the assembly, but such an embodiment features interchangeable disposable tips that perform the varied functions of heating and liposuction. Such a disposable tip may be PTFE or FEP coated and screwed onto the probe tip. After use the disposable coated tip is removed and disposed so that the probe cannula, handle and thermocouple may be easily cleaned and autoclaved before use with the next patient. A new disposable coated tip is employed with the next patient. In one embodiment, there are two separate disposable removable tips one shown in FIG. 8B is PTFE or FEP coated and features apertures such that it may be used for liposuction as described herein. The other removable disposable tip is shown in FIG. 8C is stainless steel and is used for heating reticular dermis for soft tissue coagulation. The disposable tips shown in FIGS. 8B and 8C both feature a threaded end that enables removable attachment to an end of a reusable cannula that is assembled together with a reusable handle and optionally reusable thermocouple(s).

### Continuous RF Power Clog Detection Signature

Detecting cannula clogs is an important part of any liposuction procedure. Normally, clogs happen when movement of fluid and fat within the transparent tubing becomes dormant. When the apertures are exposed to ambient pressure within the room by removing the probe from the body, the pressure differential might remove the clog and the hissing vacuum will become audible. In cases of larger clogs, the cannula may have to be flushed with saline, sucked out or pushed out to remove the clog and continue.

With the addition of continuous RF power, clogs are more important because if a clog occurs and the RF is being applied, that tissue may be further congealed or solidified into the tip of the cannula and become an irreversible adherent piece of tissue that may be difficult or impossible to fully remove/clean. When the clogs occur, it would be ideal to sense the change in flow within the cannula and/or tubing, but practically this is not possible. The flow between the body and the canister is the place to measure, but this is a messy conduit and difficult to get a good, reliable signal. Perhaps with an external ultrasound or optical flow meter such a measurement would be possible, but still difficult and expensive. The clean flow after the canister to the console is buffered by the volume of the canister and one loses the signal fidelity making it harder to detect changes in flow. For example, the vacuum pressure that is applied to the cannula is maintained by the large volume of the canister, so small changes in flow on the other side (direct to liposuction probe) are not easily detectable.

In one embodiment, impedance changes in the probes are measured to detect clogs during liposuction. For example, at the onset of the procedure, with recently tumesced tissue and a fresh probe, the measured impedance is expected to be at its lowest. As the procedure advances, the measured impedance will go up slightly and so the power will drop. Significant impedance changes and consequent power drops are likely the result of and are indicative of a clogged probe.

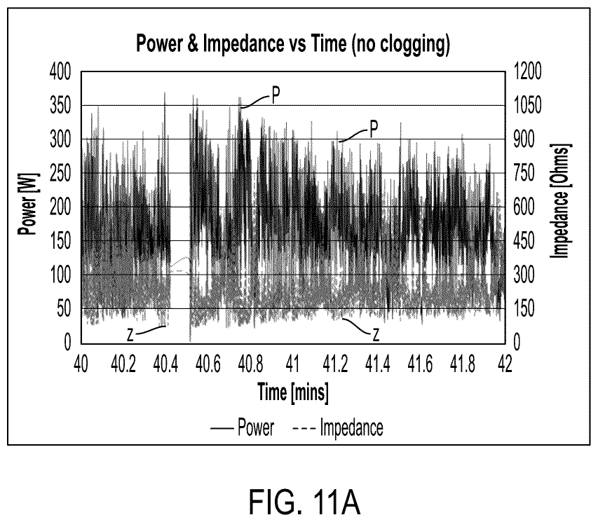

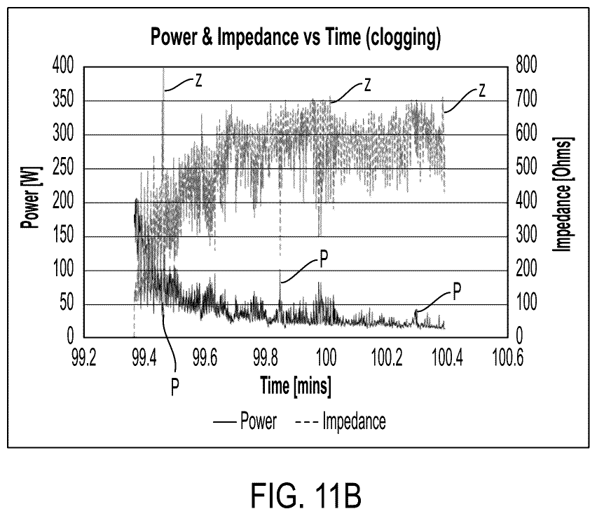

Typical power versus time and impedance versus time curves are shown in the following FIGS. 11A and 11B for two distinct cases. In the first case, FIG. 11A, the probe tip is not clogged. FIG. 11A shows Power and Impedance versus Time with no clogging. The upper portion of the plot shows power (P) and the lower portion of the plot shows impedance (Z) in FIG. 11A. In FIG. 11B, which shows clogging during continuous RF power delivery liposuction, the power data points (P) are on the lower portion of the plot and the impedance data points (Z) are on the upper portion of the plot. The power P is indicated as shown and the values are read from the left side Y-axis. The impedance Z is indicated as shown and the impedance values are read from the right-side Y-axis.

Variations in both power and impedance are visible and are to be expected as impedance conditions at the probe tip are continuously changing as the tip moves through the anatomy and as aspirated material moves through the tip. Intermittent spikes in impedance can be observed which may be explained by aspirated tissue becoming stuck intermittently (reversible clog) in an aperture or inside the tube. When this happens, fluid flow is reduced, and the local temperature will rise more rapidly. This may lead to the desiccation (drying) of the stuck tissue and/or the formation of vapor bubbles which may both account for the observed intermittent rise in impedance. When the tissue becomes unstuck, fluid flow is restored, and the impedance returns to the baseline value.

FIG. 11B shows Power and Impedance versus Time with clogging. Specifically, FIG. 11B shows characteristic curves for power and impedance when an irreversible clog has occurred. A possible explanation for the occurrence of irreversible clogs can be, for example, aspirated tissue becomes stuck in an aperture or in the tube, the suction force is insufficient to remove the clog, continued application of RF energy coupled with reduced fluid flow causes a more rapid rise in temperature leading to vapor bubble formation and desiccation of the tissue which in turn leads to a rise in impedance and an accompanying reduction in delivered power.

Where a clog remains in place, the rise in impedance and reduction in power persists. This behavior measured and shown in FIG. 11B may be used as a means to detect a clog. Specifically, the system may keep track of a rolling average impedance value and may store a system generated “baseline” rolling average value from the initial few minutes of the treatment where clogging may be less prevalent. If a rise in the rolling average is detected above a predefined level, or above the aforementioned system generated “baseline” level, the system may alert the user to check the probe tip and clean if necessary due to a suspected clog.

Similarly, a sustained period of time with an impedance level above a predefined level or the “baseline” level may trigger an alert to the user that a clog is suspected. Any algorithm for generating user alerts around clogging should not be triggered by the expected intermittent rises in impedance which are caused by intermittent clogging events such algorithms should be sensitive to clogs that are difficult to clear, lasting clogs, and/or irreversible clogs.

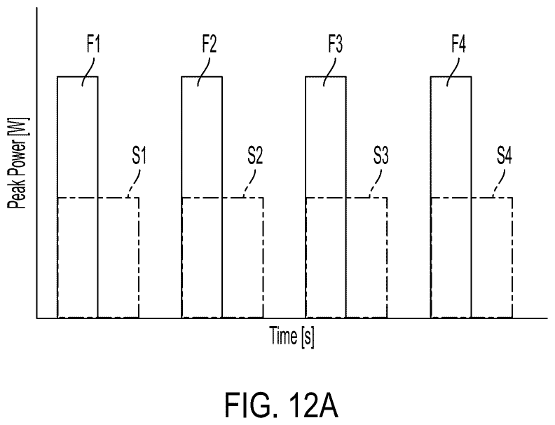

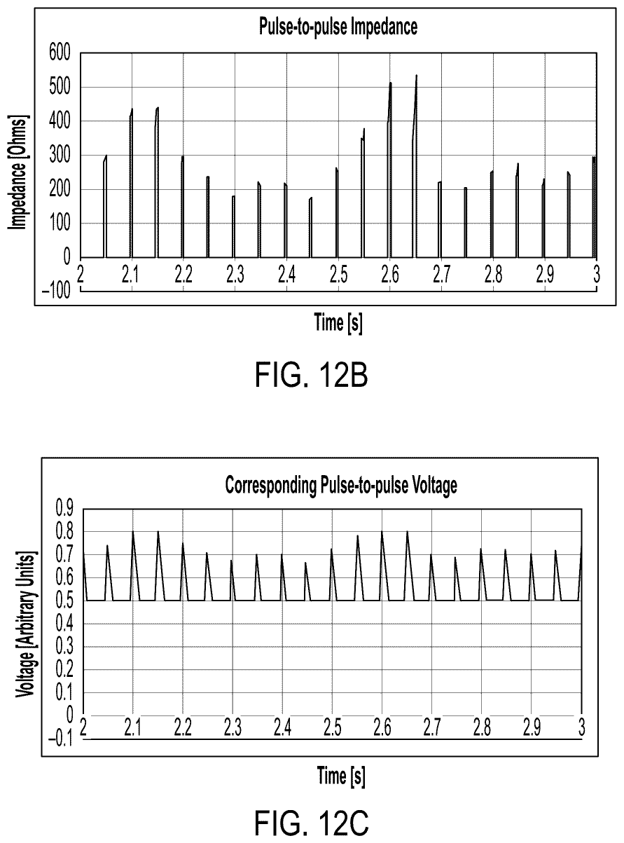

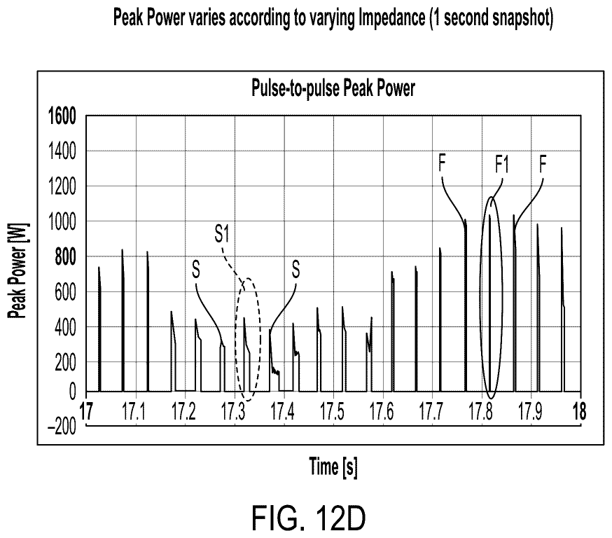

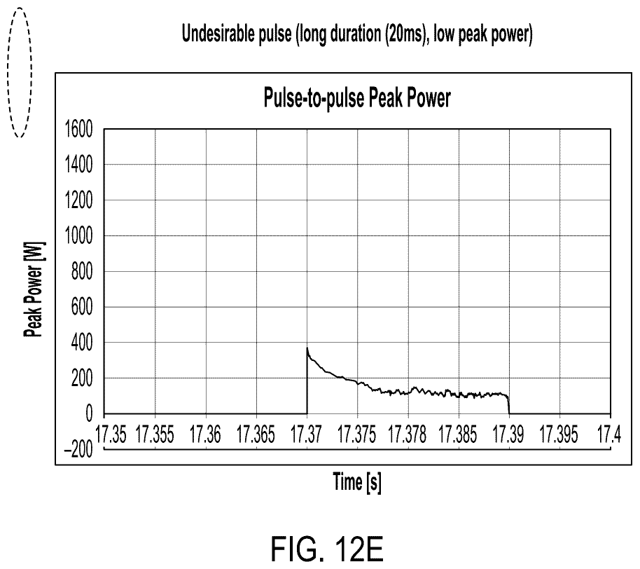

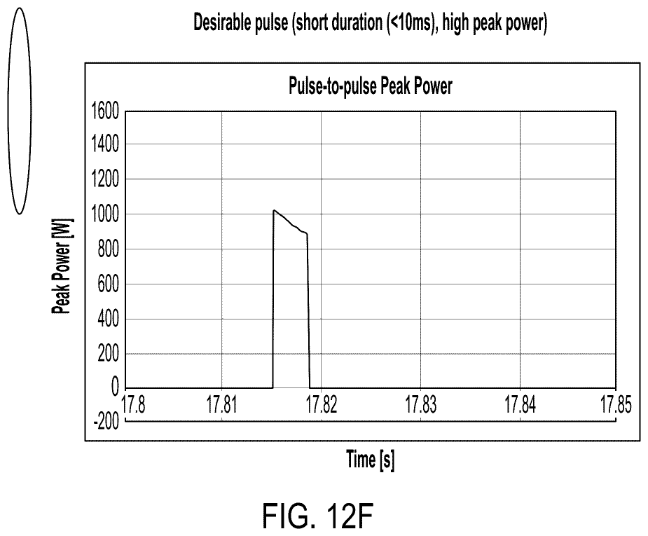

FIG. 12A provides a plot showing peak power in Watts as the y-axis versus time in seconds as the x-axis and two separate approaches to pulsed RF energy mode where the higher peak power shorter duration pulses (F1, F2, F3, and F4) shown in solid line is plotted in contrast to lower peak power longer duration pulses shown in dashed line (S1, S2, S3, and S4). As a shorthand, F refers to the faster/shorter duration pulses and S refers to the slower/longer duration pulses. The higher peak power shorter duration pulses F1, F2, F3, and F4 provide better thermal confinement than the lower peak power longer duration pulses S1, S2, S3, and S4. Of note in the FIG. 12A plot is that the higher peak power shorter duration pulses shown in solid line have the same average power as the lower peak power longer duration pulses shown in dashed line. The improvement in thermal confinement relates to the peak power rather than a comparison of average power. Further, relatively lower impedance tissue is more favorable to the higher peak power shorter duration pulses whereas relatively higher impedance tissue is favorable to the lower peak power longer duration pulses. In FIG. 12D, an exemplary less desirable S pulse is emphasized with a dotted vertical ellipse surrounding it. In contrast, in FIG. 12D, an exemplary more desirable F pulse is emphasized with a solid vertical ellipse surrounding it. Further as shown, in FIG. 12D, the exemplary undesirable pulse S1 is shown relative to other undesirable S pulses to its left and right. Similarly, in FIG. 12D, the exemplary desirable pulse F1 is shown relative to other desirable F pulses to its left and right,

A challenge of the cosmetic tissue treatment systems, devices, and methods disclosed herein is that tissue impedance is variable throughout a subject's body. Moreover, the subject's tissue impedance varies within a given treatment region targeted for treatment with the disclosed devices, systems, and methods. Because tissue impedance variability is unavoidable, uncontrolled, and to be expected, methods of managing varied tissue impedance have been built into the disclosed devices, systems and methods. For example, system voltage may be adjusted in response to the impedance measured in a treatment region during the course of a single treatment. In one embodiment, the applied voltage is automatically adjusted in real time based on locally instantaneously detected impedance measurement.

FIG. 12B shows a plot of pulse-to-pulse impedance with impedance measured in Ohms on the y-axis versus time measured in seconds on x-axis. FIG. 12C shows a plot of pulse-to-pulse voltage with voltage measured in arbitrary units on the y-axis versus time measured in seconds on the x-axis and these measurements correspond to the pulse to pulse impedance shown in FIG. 12B. FIGS. 12B and 12C show that voltage is adjusted within a range of 0.5 to 0.8 arbitrary units. The relationship between impedance and voltage in FIGS. 12B and 12C show that a higher impedance corresponds to a higher voltage and a lower impedance corresponds to a lower voltage. The disclosed approach to automatically adjust voltage based on local impedance measurement will enable the RF to output the maximum peak power within the limitations of the system. Further, it will enable the achievement of highest possible peak power at high impedance while reducing excess heating from long duration pulses. Power delivery will be more consistent. Patient to patient impedance variability and impedance variation within a single patient will be accommodated and factored into the duration of a treatment. In this way, voltage will be varied to maintain a peak power across a given impedance range. The expected voltage range and expected impedance will vary according to the probe utilized in a given treatment. Thus, where the examples provided in FIGS. 12B and 12C correspond to a probe cannula having four aperture holes such as is shown in FIGS. 5F-5I, the voltage ranges utilized in a given system would likely scale with the configuration of the probe utilized to treat a given tissue region. For example, the voltage range setpoint and impedance range of the system may be scaled up in a treatment system or method having eight aperture holes and/or in a system having four aperture slots that have a greater aperture surface area as compared to the four aperture holes depicted in FIGS. 5F-5I.

FIG. 12D is a plot of pulse-to-pulse peak power over one second of treatment with peak power measured in Watts on the y-axis versus time measured in seconds on x-axis. This shows that peak power varies according to varying impedance over this single one second snapshot of measured treatment. During this single second of treatment both an undesirable pulse S1 indicated by a dotted line and a desirable pulse F1 indicated by a solid line are present. The pulses shown as desirable and undesirable in FIG. 12D are also discussed relative to the generalization shown in FIG. 12A. FIG. 12E is a plot of pulse-to-pulse peak power over the course of the single undesirable pulse indicated by a dotted line and shown also in FIG. 12D with peak power measured in Watts on the y-axis versus time measured in seconds on x-axis. In other words, FIG. 12E depicts a magnified region of the pulse denoted by the dotted line of 12D. The undesirable pulse has a relatively long duration of 20 ms and a low peak power. FIG. 12F is a plot of pulse-to-pulse peak power over the course of the single desirable pulse indicated by a solid line and shown also in FIG. 12D with peak power measured in Watts on the y-axis versus time measured in seconds on x-axis. In other words, FIG. 12F depicts a magnified region of the pulse denoted by the solid line of 12D. The desirable pulse has a relatively short duration of less than (<) 10 ms and a high peak power.

FIGS. 12D-12F depict that pulse durations vary based on impedance conditions. Tissue impedance variability is unavoidable, uncontrolled, and to be expected. Because a longer pulse leads to a hotter treatment that is unwanted and not thermally confined, the system can set a pulse duration limit of less than 15 ms, less than 10 ms, less than 7.5 ms, or less than 5 ms, for example. Then when the pulse duration limit is met the system will discontinue treatment of a specific tissue area having undesirable conditions until it is moved away from the undesirable conditions.

Detection of the presence of high tissue impedance could be employed to signal to the system or to the practitioner that there is a presence of fibrous tissue such as scar tissue or cellulate, for example. If the system detects the presence of high impedance together with a drop in suction, then this might signal to the system or the practitioner the presence of a clog in the system.

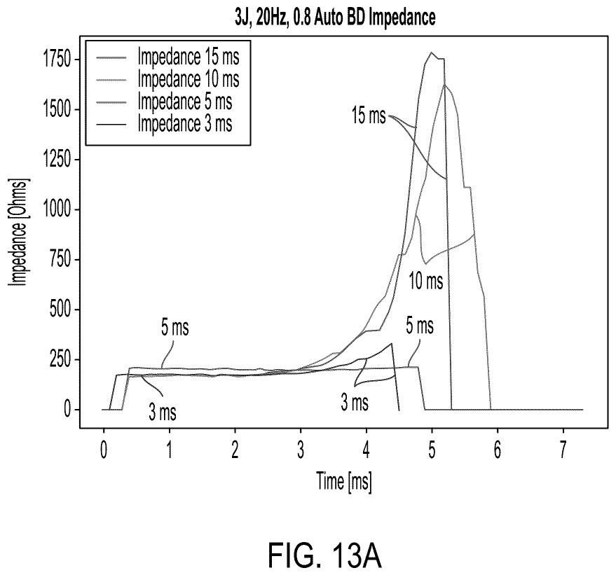

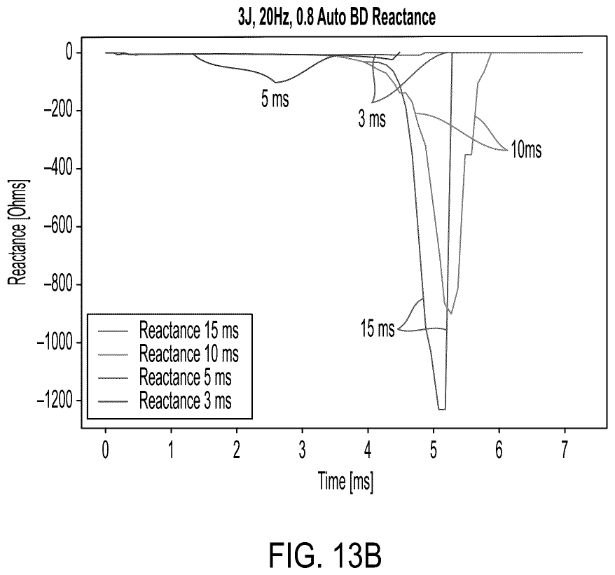

FIGS. 13A and 13B depict a proposed algorithm for improvement of plasma detection and suppression. Where excess sustained plasma forms at apertures of a probe cannula the coating disposed on the outside surface of the cannula may be damaged. The disclosed algorithms can be described with reference to FIG. 13A, which shows a plot of impedance measured in Ohms on the y-axis versus time measured in ms on the x-axis and FIG. 13B, which shows a plot of reactance measured in Ohms on the y-axis versus time measured in ms on the x-axis.

In a pulsed RF energy system plasma detection can be based upon power. If power drops rapidly with a great rate of change plasma may be present. In pulsed RF energy system plasma detection can be based upon impedance, for example, where impedance measured greater than 1000 Ohms and/or if impedance increases rapidly with a great rate of change. In pulsed RF energy system plasma detection can be based upon reactance, where reactance measured is less than about −200 Ohms and/or if reactance rate of change drops rapidly with a great rate of change.

Measuring reactance can offer a reliable indication about the presence of a plasma enabling plasma detection. Reactance results due to the formation of an electric field and formation of an electric field is indicative of formation of plasma. When plasma formation is indicated, power delivery may be cut off or stopped to terminate plasma formation. Avoiding the presence of plasma formation or the risk of plasma formation can be aided by creating a control to cut off power when the reactance hits the cut off measurement of −200 Ohms whereby the system cuts off when about −200 Ohms is detected. In another embodiment, plasma formation or the risk of plasma formation can be aided by creating a control to cut off power when the reactance hits the cut off measurement of −100 Ohms whereby the system cuts off when −100 Ohms is detected. In one embodiment, a reactance cut off is employed to determine that plasma risk is present and that there is a risk of damage to a given device if it is used below the reactance cut off.

In some embodiments, plasma is avoided such that a desirable pulse has no drop in power and no spike in impedance during the pulse. Referring again to FIGS. 13A and 13B, at 3 ms and 5 ms the impedance is relatively flat and lower than 300 Ohms and the reactance does not reach −50 Ohms, thus plasma is avoided and there is no appreciable spike in impedance during either the 3 ms or 5 ms pulse. Referring still to FIGS. 13A and 13B, at 10 ms there is an appreciable spike in impedance that is well over 1000 Ohms and peaks at about 1500 Ohms and here the reactance falls well below −200 Ohms with a low of being less than −800 Ohms here plasma is indicated. Finally, still referring to FIGS. 13A and 13B, at 15 ms there is an appreciable spike in impedance that is well over 1000 Ohms and peaks at about 1700 Ohms and here the reactance falls well below −200 Ohms with a low of being about −1200 Ohms, here plasma is indicated. Providing a reactance cut off at −200 Ohms, or −100 Ohms, lessens the risk of plasma formation during system operation thereby avoiding risk of damage to the probe cannula, cannula apertures, and/or coating(s) that plasma formation may risk.

In some embodiments of pulsed RF energy applications, the presence of plasma may be desirable. For example, in some embodiments, where the presence of plasma is desired one can determine the presence of plasma based upon a rapid drop in power, for example, with a great rate of change of power. The presence of plasma may be detected where impedance measured is greater than 1000 Ohms and/or where impedance increases rapidly with a great rate of change of impedance. In pulsed RF energy system plasma detection can be based upon reactance. The presence of plasma may also be detected where the reactance measured is less than −100 Ohms, or less than −200 Ohms, and/or if reactance rate of change drops rapidly with a great rate of change. In those embodiments where the presence of plasma may be desirable, this method of detection could allow for a degree of control over the duration of plasma exposure. For example, once plasma is detected, a time delay could be introduced such that the pulse would terminate after 1 ms, or after 2 ms, or after 3 ms. In this way, a degree of control over the time duration of the plasma could be realized.

In order to detect plasma one may monitor changes in impedance, changes in reactance, and/or changes in power. Further, plasma may be detected by monitoring one or more, or two or more of changes in impedance, changes in reactance, and changes in power. Bubble formation may be detected by monitoring one or more, or two or more of changes in impedance, changes in reactance, and changes in power. More specifically, bubble formation may be detected by monitoring one or more, or two or more rates of changes in impedance, rates of changes in reactance, and rates of changes in power. The behavior of these parameters provides an electrical signature related to the successful formation of bubbles. These electrical signatures can be employed to detect bubble formation as a closed loop to have the system react automatically in a way that keeps us in a “sweet spot” for bubble formation, i.e. the system would automatically adjust parameters to achieve and maintain bubble formation under changing anatomical conditions as identified by the electrical signature of the device.

### Pulsing RF Energy Delivery Features and Embodiments for Liposuction

For some embodiments, there may be advantages to using a pulsing RF energy delivery-based system instead of continuous wave RF delivery. In addition, in some embodiments a combination of pulsing RF and continuous wave RF delivery may be provided according to different delivery modes, tissue regions, and treatment types.

FIG. 6C includes schematic diagrams of Steps a, b, c, and d showing a perspective view of a cannula in various stages of the application of RF energy and associated fluid and bubble changes according to an exemplary embodiment of the disclosure. More specifically, FIG. 6C depicts a schematic diagram of the probe 500 being pulsed in adipose tissue. Step a shows an unenergized probe 500 in tissue without any energy being applied to the probe. The cannula of the probe 500 is placed into the adipose tissue, but RF is not applied. An aperture A having designation 530 is defined by a portion of the wall of the cannula as shown. The arrows extending outward from one end of the cannula indicates suction pressure which may be constant or selectively controlled. Step b shows the aperture A 530 on the probe 500 where pulsed RF energy is applied thereto. Upon application of pulsed RF energy, the aperture A on the probe has high instantaneous current density creating localized heat around the aperture, e.g., around the edge of the aperture EA shown by designation 540. As shown in step b, RF energy is applied to the edge of the aperture (EA) creating a high current density 540. This high current density 540 causes rapid heating and an expansion of the fluid creating a bubble (B) as shown by the four outwardly expanding arrows in Step c.

Step c shows bubble (B) formation resulting from rapid vaporization of one or more of water, fat tissue and/or tumescence present in the region of the aperture A. The formation and collapse of the bubble B contributes to the breakdown of fat and the fragments thereof (F) are aspirated into the aperture of the cannula as shown in Step d. In Step d, the five inwardly directed arrows show the movement of lysed fat fragments into the aperture in response to the collapse of the bubble and the ongoing suction. Step d shows that the expansion and collapse of the bubble in Step c causes a localized disruption that disrupts, breaks apart, or breaks down fat tissue, which is then sucked into the aperture A 530 and thereby removed via suction from the treatment region. In some embodiments, steps a, b, c, and d of FIG. 6C occur during FIG. 6B Step 2.

FIG. 6C includes schematic diagrams including Steps a, b, c, and d that show a perspective view of a probe 500 cannula in which an unenergized probe is inserted into tissue between pulses, upon introduction of pulsed RF energy an aperture with high instantaneous current density creates localized heat around the aperture, bubble formation results from rapid vaporization of water, fat and/or tumescence, and the expansion and collapse of bubbles disrupts fat and the fat is sucked away. The Steps a, b, c, and d of FIG. 6C occur during FIG. 6B Step 2. More specifically, FIG. 6C depicts a schematic diagram of the RF probe reducing the volume of a tissue area of unwanted adipose tissue via a debulking liposuction step described in association with FIG. 6B Step 2.

In most embodiments, a given cannula defines multiple apertures along its length. This process is repeated at a repetition rate from about 1 to about 1000 Hz, preferably at 20 Hz and the multi-aperture cannula is reciprocated through the tissue at a rate of 1-10 cm/sec many times to remove adipose tissue evenly and effectively. In some embodiments, a given probe or treatment device, such as a cannula-based device, may include one or more detectors. In some embodiments, a given probe or treatment device or liposuction system may include a temperature sensor and an acoustic/sound sensor. The various sensors may be disposed on or in a given treatment device or otherwise be positioned relative to a given treatment area. Various other sensors may be used such as motion sensors, accelerometers, pressure sensors, sound sensors, force sensors and others.

The disclosed pulsed RF energy delivery for liposuction may be used to initiate tissue changes in fatty tissue by introducing bubbles (e.g., steam bubbles) into the fatty tissue. Generating steam bubbles via pulsed RF energy requires a minimum threshold amount of energy density compared to methods that introduce RF energy via continuous wave energy density to heat fatty tissue. At a high level, with regard to the various steps and stages shown in FIG. 6C, in a pause or delay in which the probe is unenergized between pulses, localized heat is created around aperture(s) in response to high instantaneous current density of, around, or from the aperture, forming bubbles in response to one or more state changes such as rapid water/fat/tumescence vaporization, and expanding and collapsing of one or more bubbles that disrupt fat, which is then suctioned into the probe for disposal or other cosmetic uses.

The liposuction cannula provides pulsed RF energy delivery that generates bubbles in fatty tissue. Bubble expansion, collapse and associated pressure or shock waves break the cell membranes of some of the fat cells and this appears to make it easier for the disrupted fatty tissue to be extracted via the liposuction cannula. In contrast to continuous wave RF energy delivery, the generation of bubbles via pulsed RF energy delivery requires less energy per unit volume of tissue delivery and unlike the continuous wave approach does not require the whole volume of extracted fatty tissue to have been heated.

Benefits of pulsed RF energy delivery include that the generation of steam bubbles in fatty tissue appears to enable a higher volume of fat to be removed via cannula (e.g., more efficient removal of fat) than via a continuous wave (“CW”) approach that requires all of the fatty tissue being removed to be heated. As a result, there is potential for a shorter treatment time via pulsed RF than via CW RF power delivery for liposuction. Further, where pulsed RF is employed to generate steam bubbles, less energy density is applied to fatty tissue to achieve a given result, and so it is expected that unwanted heat related side effects can be lessened and/or avoided.

In some embodiments, bubbles may be generated via pulsed RF. Without being held to a particular theory or mechanism, in some embodiments, heat confinement in the area near the electrode where the bubbles are formed may initiate, cause, or support bubble generation.

For pulsed RF treatment, sufficient RF power is needed for energy delivery. As such, a pulsed RF device is more complex to build versus a CW RF power device, because of the required increase in peak RF power delivery during pulsed on-time. Specifically, for a CW RF treatment a device delivering between 50 watts and about 300 watts may be sufficient, whereas, for pulsed RF a device delivering between about 1000 watts and about 3000 watts, or about 1600 watts pulsed for from about 3 to about 60 milliseconds, from about 5 to about 30 milliseconds, from about 10 to about 50 milliseconds, from about 3 to about 20 milliseconds, or from about 20 to about 30 milliseconds may be employed.

In one embodiment, the practitioner may be presented with a GUI screen with energy delivered per pulse setting and the pulse may have variable energy from between about 1 and about 20 Joules, from about 2 to about 15 Joules, and from about 5 to about 10 Joules. The pulsed RF treatment approach may provide feedback to the practitioner regarding successful bubble formation due to pulsed RF the feedback being in the form of an audible sound of a bubble and/or a tactile feeling of a bubble felt from palpating the exterior skin of a patient or from a sound level detected by a piezoelectric transducer present on either the cannula or on the exterior surface of the skin of the subject. In some embodiments, the initiation of the sound of the bubble provides clinical set point information to the practitioner who after starting at relatively low energy delivery per pulse raises the energy delivery level until the bubble feedback is received.

More specifically, bubble formation may be detected by monitoring one or more, or two or more rates of changes in impedance, rates of changes in reactance, and rates of changes in power (as disclosed in FIGS. 12A-12F and 13A-13B). The behavior of these parameters provides an electrical signature related to the successful formation of bubbles. These electrical signatures can be employed to detect bubble formation as a closed loop to have the system react automatically in a way that keeps us in a “sweet spot” for bubble formation, i.e. the system would automatically adjust parameters to achieve and maintain bubble formation under changing anatomical conditions as identified by the electrical signature of the device.

In some embodiments, upon detection of bubble feedback, (e.g., energy per pulse that achieves the desired bubble formation) the practitioner can begin the treatment regime in a given region of the subject's body with the desired/appropriate energy delivery per pulse. In some embodiments, the sounds associated with a bubble forming or popping or otherwise associated with a change in sound with fat fragmentation or another sound of interest may be detected with a sound detector. In various embodiments, the sounds detected by the sound detector may be converted into a visual alert or used to generate other visual reports, automatic control signals, or other events of interest and correlated system actions. Spatial uniform energy density deposition via pulsed RF energy over the volume of the treatment area helps to avoid undesirable outcomes like uneven treatment for many embodiments.

In some embodiments, pulsing RF delivery may be suitable for generating steam bubbles in aqueous media, materials and tissue. A schematic showing the method of action of pulsed RF power applied to liposuction probe apertures is shown in FIG. 6C Steps a-d. In other applications, pulsing at energy levels that are below bubble formation thresholds would be capable of fractionally coagulating tissue as the probe is moved through tissue. Some pulsing-based RF delivery methods may support detection of a threshold or transitional event upon the occurrence thereof allows a user to be aware of or alerted to an RF-tissue interaction, such as through a popping sound or other noise or alert. A threshold energy level may be achieved during which a consistent amount of popping or other noises or alerts may be achieved. In various embodiments, the energy delivered may be increased incrementally or marginally such as about 10% to about 20% above the threshold level associated with the noise or other alert to reach a target endpoint or other target while not adding unnecessary heat to the subject.

Delivery of RF energy in aqueous media generally leads to heating of the media in the vicinity of the RF electrode(s). When RF power is sufficiently high, the heating of the aqueous media may lead to generation of steam bubbles and plasma. The process can be utilized for various applications in aesthetic medicine. The following considerations highlight important parameters of the process under a few simplifying assumptions.

Laser energy assisted lipolysis is a well-established technique. Laser assisted lipolysis is practiced both with repeatedly pulsed devices as well as with Continuous Wave (“CW”) devices (usually based on diode lasers). At sufficiently high pulsed peak power the pulsed laser devices can generate steam bubbles in the adipose tissue while delivering moderate average power, for example 5W to 50W. Existing RF assisted lipolysis devices available on the market deliver RF power at levels that heat the adipose tissue without any consideration for delivering sufficiently high peak power to create steam bubbles in the adipose tissue. The use of the wet infiltration technique in the adipose tissue utilizing infiltration with a solution containing 2% lidocaine with vasoconstrictor in warm saline solution is a commonly used local anesthesia technique that may be used with laser assisted lipolysis [See Badin, Ana, et al. “Laser lipolysis: flaccidity under control.” Aesthetic plastic surgery 26:335-339, (2002)].

The commonly used anesthesia by infiltration of the adipose tissue with a saline solution can provide a medium suitable to an approach disclosed herein however where an RF assisted lipolysis technique in which the RF power is repeatedly pulsed, but unexpectedly, leads to generation of steam bubbles in the aqueous media composed of the adipose tissue with saline solution, referred to as the intermixed media. The proposed technique employing formation of steam bubbles in the aqueous media will lead to generation and propagation of pressure waves and possibly shock waves originating from the steam bubbles. The pressure wave's propagation through intermixed media is affected by the value of each medium's acoustic absorption and impedance. The acoustic absorption is directly related to protein content, and acoustic absorption is substantially larger in fatty tissue vs water like saline solution [See Shankar, Hariharan, at al. “Potential adverse ultrasound-related biological effects: a critical review.” The Journal of the American Society of Anesthesiologists 115.5:1109-1124, (2011)]. Steam bubble formation, bubble expansion and pressure wave propagation in the adipose tissue is expected to lead to selective thermo-mechanical damage to fat cells and surrounding fatty, fibrous or other tissues while limiting the damage to vascular structures in the treated anatomical site.

The bulk dielectric properties of fatty tissue are documented over extended frequency range [See Gabriel, Sami, R. W. Lau, and Camelia Gabriel. “The dielectric properties of biological tissues: III. Parametric models for the dielectric spectrum of tissues.” Physics in medicine & biology 41.11:2271, (1996)]. On a microscopic scale fatty tissue is inhomogeneous with pockets of fat cells surrounded by fibrous tissue and blood vessels leading to spatially varying electrical impedance. The infiltration of the adipose tissue with a saline solution typically leads to inhomogeneous saline intermixing and increases the spatial variation of the electrical impedance.

Operator controlled movement of a RF delivery cannula with one or more annular electrodes through the fatty tissue will lead to varying electrical impedance in the electrical circuit delivering the RF power.

### Pulsing RF Energy Delivery Features and Embodiments Probe Heating/Tightening

FIG. 6D depicts the method of action to create thermally confined heated zones near the reticular dermis. As the cannula of the probe 500 is passed through the tissue in stroke direction 611 the aperture(s) 530 are energized by RF sufficiently (such as through an RF signal with one or more selected probe parameters) to heat and coagulate regions 645 of adjacent dermal tissue. The pulse frequency and cannula speed determine the density of the lesions 645. In one embodiment, using moderate RF pulsed power, e.g., less than 200 W, RF pulsing in energy control mode is expected to be able to produce multiple stripes or multiple toroidal like regions of coagulated tissue 645 at the dermis/fat junction while delivering relatively low average power over the treated region. The overall skin response to the described multiple coagulation injuries 645 and the general dermal heating is expected to lead to a healing response that may include the desired skin tightening effect similar to what may be achieved with a 1440 nm repetitively pulsed laser.

FIG. 6D includes schematic diagrams including Steps a, b, c, and d that show a perspective view of a probe 500 cannula creating a series of coagulation zones near the reticular dermis and/or at the dermis/fat junction. The Steps a, b, c, and d of FIG. 6D occur during FIG. 6B Step 3. More specifically, FIG. 6D depicts a schematic diagram of the RF probe heating/tightening dermal tissue subsequent to volume reduction via liposuction described in association with FIG. 6B Step 2 and FIG. 6C.

FIG. 6D step a shows a probe 500 having one or more apertures 530 in tissue without any energy being applied to the probe between pulses. Step b shows the one or more apertures 530 at the instant or the point in time when a pulse of RF energy is applied to the exposed stainless steel 540. The aperture 530 with the exposed stainless-steel edge 540 is energized by a pulse of RF energy. Upon application of pulsed RF energy, the aperture 530 on the probe has high instantaneous current density creating localized heat around the aperture, e.g., around the edge of the aperture EA 540. Step c shows that the high current density causes rapid heating that induces coagulation injuries 645 as the probe moves in the direction 611 of the stroke (e.g., forward). Step d shows a series of coagulation zones 645 also called coagulation injuries 645 formed in the reticular dermis tissue region. In various embodiments, the Steps a, b, c, and d of FIG. 6D occur during FIG. 6B Step 3. More specifically, FIG. 6D depicts a schematic diagram of the RF probe heating/tightening dermal tissue after volume reduction via liposuction described in association with FIG. 6B Step 2 and FIG. 6C.

### Energy Delivery Management

A typical generator of RF power controls the amplitude or the RMS value of the delivered voltage (V). It is less common to have RF generator that controls the amplitude or the RMS value of the delivered current (I). In adipose tissue with spatially variable impedance (R), the typical voltage control RF generator will deliver spatially variable power per unit volume P=V²/R (or for the current control generator, the spatially variable power per unit volume is P=I²R). The delivery of spatially variable power may lead to inconsistent levels of adipose tissue damage and inconsistent clinical effects.

One possible way to compensate for the spatially variable impedance of the infiltrated adipose tissue is to use a RF generator with real-time voltage or current control that adjust the voltage or current level so that it delivers substantially uniform spatially distributed power per unit volume for a range of tissue impedance values. The design of such uniform power per unit volume real-time voltage or current control RF generator is more difficult and would result in a more complicated, more expensive device.

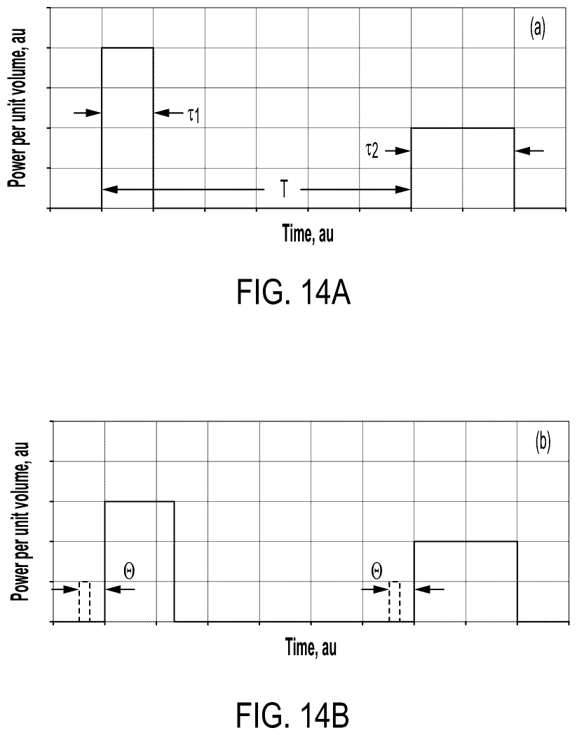

An alternative approach is proposed where the goal is to deliver substantially uniform spatially distributed energy per unit volume of adipose tissue, Q, by delivering the variable power per unit volume P over variable periods of on-time t. The control system of the example voltage control device varies in real time the RF pulsed on-time t so that delivered energy density per unit volume Q=(V²/R)τ is substantially uniform for a range of impedance values between 50 and 500052. The control system may deliver preset energy density per unit volume Q by delivering lower power per unit volume for longer on-times, τ, in regions with high impedance, R; and by delivering higher power per unit volume for shorter on-times, τ, in regions with low impedance, R. For example, FIG. 14A shows the delivery of a preset energy density per unit volume by delivering higher power per unit volume for shorter on-time, τ1, in region with lower impedance; and delivering lower power per unit volume for longer on-time, τ2, in region with higher impedance. The delivery of consistent energy density per unit volume Q, using variable power per unit volume over real-time adjustable variable periods of on-time τ can be repeated at a time interval T, with variable or fixed repetition rate, f=1/T, between about 1 and about 1000 Hz. The delivery repetition rate can be adjusted so that the average power per unit volume delivered in the treated anatomical region, Pav=Q f, is maintained within clinically safe and effective limits.

In some embodiments, the control system sets the output control parameter, for example the rms voltage V, to be approximately the same for each pulse during the treatment. In other embodiments, for each pulse during the treatment the control system delivers a lower voltage impedance-diagnostic short pre-pulse. Then the control system calculates the local impedance for the RF delivery site and sets the output voltage so that it optimized the RF power delivery during the treatment pulse for the measured local impedance. For example, FIG. 14B shows the impedance diagnostic short pre-pulses with a dashed line. Based on the measured local impedance, the delivered peak power density is decreased and in FIG. 14B example the first treatment pulse is showed with a decreased peak power density and increased on time vs the first pulse in FIG. 14A so that the delivered energy density, the area under the curve, is maintained the same. It is preferable that the pre-pulse time interval between the impedance-diagnostic short pre-pulse and the treatment pulse is kept relatively short, labeled as Θ in FIG. 14B.

In some embodiments, the practical range for the pre-pulse time interval is the speed of motion of the RF treatment cannula held in the operator's hand and the size of the annular RF electrodes. An example estimate can be based on the approximately 5 m/s hand speed reported in [See Elgendi, Mohamed, et al. “Arm movement speed assessment via a Kinect camera: a preliminary study in healthy subjects.” Biomedical engineering online 13.1:1-14, (2014)] and a 1 to 4 mm estimate for the annular electrode diameter. Then the estimated range of values for the pre-pulse time interval is between 0.2 and 0.8 ms. In further embodiments, the control system varies the output control parameter based on real-time impedance measurement while the treatment pulse is delivered.

In another example for a voltage control system, at the beginning of each on-time interval the voltage is set at a value that will deliver a safe amount of power per unit volume P=V²/R. Then the real time control system monitors the local tissue impedance and increases the control voltage, V, when it detects higher impedance R, on a time scale much shorter than the individual pulse on-time, so that the desired energy density per pulse Q=(V²/R)τ can be delivered in the desired pulse on-time, for example 1 ms to 10 ms. In a further example, in some device implementations where the available range of control voltages is limited and the delivered energy density per unit volume, Qᵥ, cannot exceed the threshold energy density for bubble formation, Qₜₕ, at the higher voltage setting, the power delivery can be terminated in order to limit the localized tissue heating without steam bubble formation, for example after about 3 ms to about 10 ms.

### RF Generation of Steam Bubbles

The generation of vapor cavities, or bubbles, near the RF electrodes in aqueous media and ionization of the vapor cavities is demonstrated to occur sequentially in time in [Palanker 2008]. The sequential nature of the process allows for separate analysis of the process of bubble formation.

A simplified model for bubble generation in aqueous media is outlined in [See Gerstman, Bernard S., et al. “Laser induced bubble formation in the retina.” Lasers in Surgery and Medicine 18.1:10-21, (1996)]. The simplified model assumes a spherical volume in aqueous media where energy is delivered in a small volume embedded in a larger volume of aqueous media without energy deposition. The model considers pulsed energy delivery with sufficiently short pulses for heat confinement and derives the constraint that bubble formation is a threshold limited process. In the adiabatic approximation, the threshold limited process for bubble formation is characterized by a threshold energy density per unit volume, Qth. Delivery of energy density below the threshold, Qth, would lead to a temperature rise in the region where energy is delivered with no steam bubble formation. Delivery of energy density, Q, above the threshold, Qth, would lead to formation of a steam bubble with volume proportional to the amount of excess energy density above the threshold, Q−Qth.

In a threshold limited process, like bubble formation, the energy utilization efficiency favors operation high above threshold. For delivered energy densities below the threshold, Qth, the energy utilization efficiency is zero—there is no generated bubble. For delivered energy densities above the threshold, Qth, the energy utilization efficiency can be defined as

$$η = \frac{{Q}_{v} - {Q}_{th}}{{Q}_{in}} .$$

Qv is the energy density that accumulates at the end of the pulse in the volume of water that may be converted into a steam bubble and Qin is the total delivered input energy density. Operation at short pulse durations that provide sufficient thermal confinement allows for the most efficient energy delivery, Qv≈Qin.

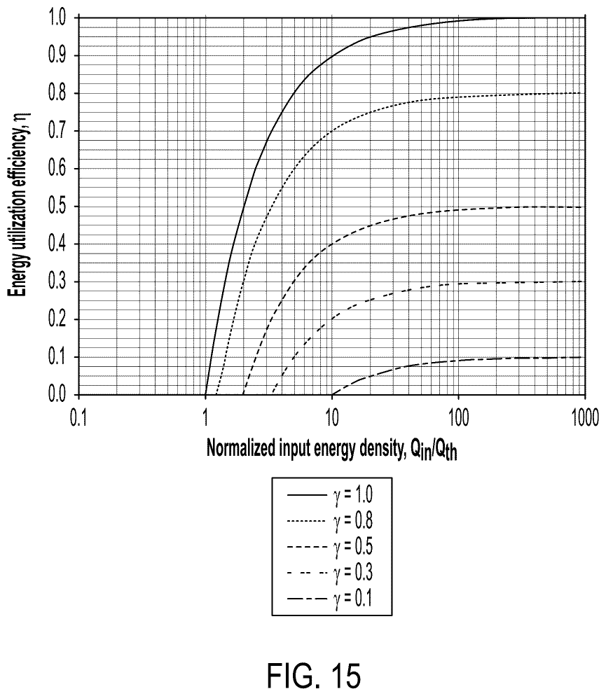

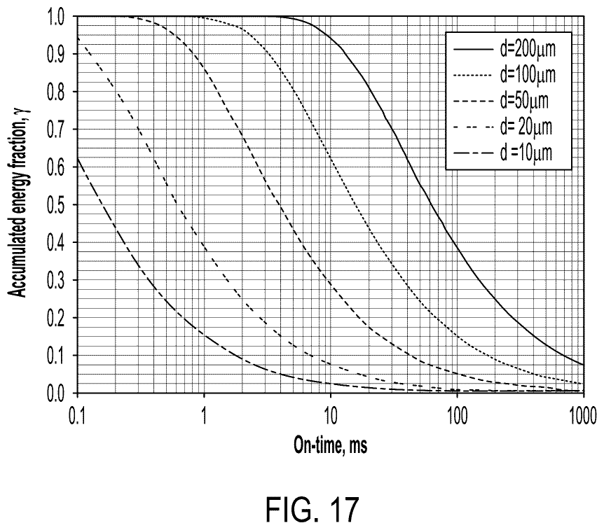

In some embodiments, operation at longer pulse durations that do not provide thermal confinement may decrease the fraction of input energy density, γ, that may accumulate at the end of the pulse in the volume of water that may be converted into a steam bubble, Qv=γQin. In the limit of very long pulses, approaching continuous operation, the fraction of input energy density, γ, may be the lowest. A low fraction of accumulated input energy density, γ, may lead to low energy utilization efficiency for fixed input energy density. Referring now to FIGS. 15 and 17, these figures both feature the accumulated energy fraction γ, and are accordingly interrelated.

The dependence of the energy utilization efficiency, η, as a function of the normalized input energy is plotted on FIG. 15 for a few fixed values of the accumulated energy fraction accumulated at the end of the pulse, γ. FIG. 15 demonstrates the advantages of modes of operation with high accumulated input energy density fraction at the end of the pulse, γ, and high input energy pulse depositing energy density high above the threshold energy density for bubble formation.

The determination of the exact geometrical shape of the volume of superheated water that may be converted into a steam bubble is not part of the present simplified analysis. In a simplified example, for an annular shaped RF electrode exposed to the aqueous media, the volume of water that may be converted into steam can be expected to have a toroidal like, or doughnut like, shape. The annular shaped RF electrode is expected to be in contact with the external surface of the toroid and possibly the toroid external surface may extend beyond the edge of the electrode. Limiting the area of the electrode in contact with the aqueous media may lead to a higher current density and more efficient generation of steam bubbles.

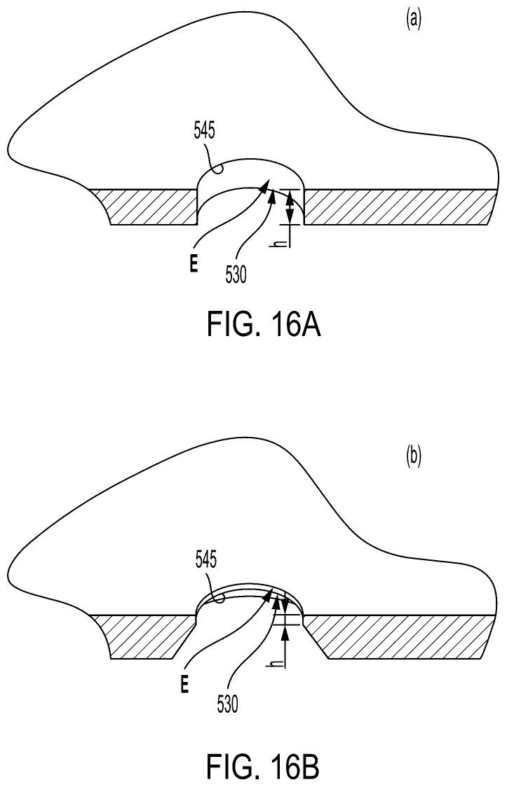

Examples of annular shaped RF electrodes or apertures 530 are shown in FIGS. 16A and 16B. When the annular shaped electrode is formed in relatively thin RF conducting material, the annular shaped electrode can be formed as a hole through the material as shown in FIG. 16A. An annular shaped electrode formed in relatively thicker RF conducting material may be formed as a tapered hole through the material as shown on FIG. 16B. In both examples the height of the annular shaped electrode, (e.g., aperture 530) shown as n on FIGS. 16A and 16B, is chosen to provide sufficiently high RF energy density deposition in the aqueous media (e.g., tissue intermixed with tumescence) in contact with the annular electrode surface shown as E in FIGS. 16A and 16B.

Multiple annular shaped electrodes may be formed in various patterns on the RF conducting material. All other surfaces of the RF conducting material that may come into contact with the aqueous media, except the annular electrode surface(s) labeled as E, should be modified to decrease their RF conductivity into the aqueous media for example by coating, plating, anodizing or any other suitable process leading to a substantial decrease in RF conductivity. Here substantial decrease in RF conductivity is defined as conductivity decrease by a factor larger than 2×, or more preferably larger than 5×, or most preferably larger than 10×.

In the present analysis the convoluted toroidal like shape of the volume of water that may be converted into a steam bubble may be approximated with a cylinder with variable diameter and length that matches the circumference of the annular shaped RF electrode. The diameter of the cylinder can be chosen so that its volume matches approximately the superheated water volume in the vicinity of the annular shaped electrode. In the approximate model the cylinders are considered to be heated by a source depositing uniform energy density in their volume.

The calculated accumulated energy fractions at the end of the pulse, γ, at the center of a uniformly heated cylindrical volumes of water with a range of diameters embedded in unheated water volume are plotted on FIG. 17. The plots are calculated for fixed deposited uniform energy density in the cylindrical volumes of water. As shown in the legend for the various curves in FIG. 17, a ranges of variable diameter cylinders with diameter between 10 and 200 μm were evaluated.

The calculated accumulated energy fractions at the end of the pulse, γ on FIG. 17, show a strong non-linear dependence vs the pulse on-time and the diameter of the heated region. The plot illustrates the advantages of short pulse on-times especially for smaller diameter of the heated region, provided the heating source has sufficient peak power to deliver the required energy density in a short on-time pulse. For very long on-times, about 1000 ms, the plot illustrates the disadvantages of continuous-like power delivery with very low accumulated energy fractions for the considered range of diameters of the heated region.

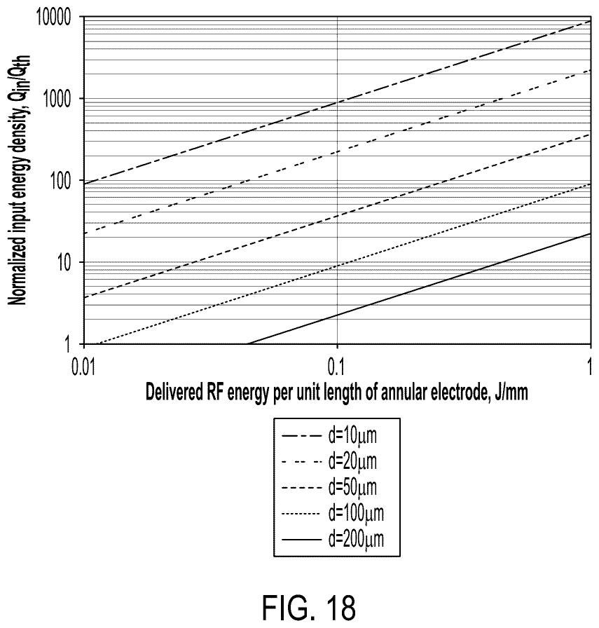

The finite amount of RF power that can be delivered over the time intervals corresponding to heat confinement in the range of considered diameters of heated region sets a practical limit for the circumferential length of the annular electrode(s). The deposited input energy density can be calculated as

$${Q}_{in} = \frac{{E}_{in}}{1 / 4 π {d}^{2} L} ,$$

where Ein is the delivered RF electrical energy and the denominator accounts for the considered cylindrical volume of heated water. In designs where multiple annular electrodes or apertures are desired, L is the total circumferential length of the annular electrode aperture(s). The value for the energy density threshold for steam bubble formation can be calculated for water as Qth=1.42 J/mm³, following the method outlined in [Gerstman 1996]. Instead of the deposited input energy density, Qin, it is more convenient to consider the normalized input energy density, Qin/Qth, used also in the energy utilization efficiency calculation. FIG. 18 is a plot showing normalized input energy density versus the delivered RF energy per unit length of annular electrode for a range of uniformly heated variable diameter cylinders embedded in unheated aqueous media. The normalized input energy density, Qin/Qth, for a range of delivered RF energy per unit length of annular electrode(s), Ein/L, is plotted on FIG. 18 for a range of diameters for the cylindrical volumes of water.

Following earlier discussion for the energy utilization efficiency, only values of the normalized input energy density above 1 may lead to generation of steam bubbles. Depending on the actual level of heat confinement and corresponding accumulated energy fraction at the end of the pulse, the normalized input energy density, Qin/Qth, that is expected to lead to steam bubble formation should be larger than about 2, or more preferably larger than about 5, or most preferably larger than 10, as plotted on FIG. 15. Larger values of the normalized input energy density, Qin/Qth, may lead to more efficient energy utilization for steam bubble formation.

In designs where electrically parallel multiple annular electrodes in isotonic saline like aqueous media are desirable, steam bubble formation at a subset of one or more electrodes facilitates more efficient bubble formation for the remaining electrodes due to the higher impedance of steam vs isotonic saline.

During the on-time of the pulsed energy delivery, the subset of one or more electrodes with early steam bubble(s) formation may receive decreased energy flow after the bubble formation while the remaining electrodes may receive increased energy flow that may increase the rate of steam bubble formation. In some embodiments, steam bubble formation in devices with electrically parallel multiple annular electrodes is a cooperative process-once steam bubble formation starts it is expected to lead to bubble formation at all annular electrodes, provided sufficient RF energy is delivered in a sufficiently short on-time. In some embodiments, preferred on-time ranges between about 0.1 ms-about 100 ms, or from about 0.5 ms-about 20 ms, or from about 0.5 ms-about 15 ms, or from about 0.5 ms-about 10 ms, or from about 0.5-about 7.5 ms.

In another example for real time control of the energy in each pulse, the detection of steam bubble formation with a piezo electric transducer can be used for termination of the power delivery in the pulse shortly after the steam bubble detection leading to delivering of real time adjustable energy per pulse. Such real time control mode of operation minimizes the pulse energy in each pulse while delivering consistent bubble formation in most pulses when the delivered peak power is sufficient for bubble formation. The minimized pulse energy in each pulse will lead to minimized average power delivered in the treated region while maintaining the treatment mode of operation based on steam bubble formation. The minimized average power delivered in the treated region will lead to improved treatment safety.

An additional feedback mechanism can be envisioned where steam bubble formation in the infiltrated adipose tissue is detected in real time and for every pulse RF power delivery is terminated at deposited energy levels about 10% or about 20% above the detected threshold for steam bubble formation. For example, steam bubble formation can be detected with a microphone or a piezo transducer connected to the delivery cannula or to the patient skin. A feedback mechanism for pulse energy control based on the formation of steam bubbles may allow treatment of adipose tissue with minimal spatially averaged energy delivery in a tissue region and improved treatment safety. In delivery devices where energy delivery continues after steam bubble formation, the delivered additional energy density might lead to ionization and plasma formation in one or more of the steam bubbles associated with the annular RF electrodes.

Adipose tissue infiltrated with a saline like solution typically has spatially varying RF electrical impedance. The delivery of substantially uniform spatially distributed energy per unit volume in the tissue with varying RF electrical impedance is facilitated by delivering the uncontrollable variable power per unit volume P over real-time controlled variable periods of on-time τ. The control system delivers preset energy density per unit volume Q=Pτ by delivering lower power per unit volume for longer on-times, τ, in regions with high impedance; and by delivering higher power per unit volume for shorter on-times, τ, in regions with low impedance.

Pulsed energy delivery of RF power in adipose tissue infiltrated with saline like solution may lead to generation of vapor cavities, or steam bubbles, for a range of deposited energy densities and on-times. Generation of vapor cavities, or steam bubbles, in aqueous media is a threshold limited process. The efficiency of generation of steam bubbles near RF energy delivery devices with multiple annular RF electrodes, each having one or more apertures, in fatty tissue infiltrated with saline like solution can be improved by delivering, through the one or more apertures, short-pulsed bursts of high peak power density that provide sufficient heat confinement and energy per unit length. Varying in real time, pulse to pulse, the on-time of the RF high peak power density bursts allows the delivery of consistent energy density per unit volume at every pulse when the RF impedance of the surrounding medium varies.

Decreasing the annular electrode size exposed to the isotonic saline like media may improve the efficiency of bubble formation by increasing the current density near the exposed electrode and decreasing the size and volume of the heated water to be converted into steam. The disclosed annular electrode may also be referred to as the perimeter of the aperture in the electrode and can have any geometric shape not limited to a shape featuring an ellipse. In a cannula like device with one or more annular electrode areas, electrical insulation on the external and internal surface of the cannula walls may be beneficial for limiting the energy deposition only to the vicinity of the annular electrode areas. When the pulsed mode of operation reliably generates steam bubbles near the annual electrode(s) in the aqueous media that is evacuated through the cannula, a high-volume density of steam bubbles may increase sufficiently the impedance of the evacuated aqueous media so that the energy loss through the uninsulated internal cannula may become less significant.

For some RF or other energy delivery embodiments, pulsed operation is employed in contrast to continuous operation. The two modes of operation differ by varying the temporal component and in the pulsed application the peak power. A minimum energy per unit time may be selected to affect the tissue, either thermally or through mechanical disruption via the bubble formation or both, and this is represented as power. There also exists an upper limit on the same application of that power. If the objective is to confine the heat to a small volume, as is the case with forming bubbles high peak powers may be used. For example, high peak powers can be used for a limited amount of time in a pulsed fashion, thus limiting the average power applied to the tissue. The RF generator may be designed to be operated at high powers for short sequential pulses and low duty cycles, but also use safe average powers.

### RF Pulsing and Localized Dermal Coagulation for Skin Tightening

Laser assisted lipolysis is practiced both with repetitively pulsed lasers as well as with CW devices (usually based on diode lasers). Early clinical and histology observations with repetitively pulsed lasers showed some skin tightening associated with superficial, subdermal deposition of laser energy using 1064 and/or 1320 nm repetitively pulsed lasers. Sec, Dibernardo, Barry et all. “Evaluation of tissue thermal effects from 1064/1320-nm laser-assisted lipolysis and its clinical implications.” Journal of Cosmetic and Laser Therapy 11, no. 2:62-69, (2009).

Subsequent introduction of the 1440 nm repetitively pulsed, side firing laser device demonstrated neck contouring and skin tightening in the neck area. See, FAAD, Deborah S. Sarnoff MD. “Evaluation of the safety and efficacy of a novel 1440 nm Nd: YAG laser for neck contouring and skin tightening without liposuction.” Journal of Drugs in Dermatology 12, no. 12: 1382-1388, (2013), and DiBernardo, Gabriella A., and Barry E. DiBernardo. “Prediction of treatment outcomes for neck rejuvenation utilizing a unique classification system of treatment approach using a 1440-nm side-firing laser.” Aesthetic Surgery Journal 38, no. suppl_2: S43-S51, (2018).

The increased absorption of 1440 nm in the dermis and in fatty tissue facilitates localized dermal tissue heating when the energy is delivered sub-dermally with a side-firing fiber delivering about 50% of the energy towards the dermis.

Existing RF assisted lipolysis devices deliver RF power at levels that heat the adipose tissue and may lead to skin tightening. Sec, Paul, M and Mulholland R S “A new approach for adipose tissue treatment and body contouring using radiofrequency-assisted liposuction.” Aesthetic plastic surgery 33:687-694, (2009). It is possible that clinical optimization of CW RF power level and temperature setting at the cannula thermistor will lead to some level of skin tightening, possibly approaching the effect of the 1064 and/or 1320 nm lasers. The 1064 and/or 1320 nm lasers deliver the optical energy coaxially with the fiber axis predominantly in the sub-dermal adipose layer. Direct laser energy delivery in the dermis is minimal, most dermal heating is by heat diffusion from the heated adipose layer. For the repetitively pulsed 1064 and/or 1320 nm lasers even though the energy delivery by the individual laser pulse is thermally confined in the adipose layer, the observed dermal temperature rise is the result of the combined effect of multiple pulses with the associated thermal diffusion during the pulse and the inter-pulse time intervals, resulting in unconfined heating of the treated dermal tissue volume. At 1440 nm the main chromophore in the lower dermis is water. The water absorption coefficient is 29.4 cm−1 at 1444 nm. The 1444 nm laser delivery fiber combines coaxial and side firing capabilities and can deliver about 50% of the pulse energy directed towards the dermis where the repetitively pulsed energy delivered in pulse widths of approximately 380 us is thermally confined.

Ex-vivo tests on excised human tissue from abdominoplasties as well in-vivo cases have led to various observations regarding the operation and performance of the devices disclosed herein. A summary of some of these observations is outlined as follows:
The larger the ID, the better the suction.
Insulation of the incision site is likely required either through an insulated incision port or an insulated cannula.
With respect to cannula apertures 530, holes (e.g., referring to FIGS. 5D and 5K) appear to perform better than slots (e.g., referring to FIGS. 5A and 5B). The larger slots may create aspirated pieces that are longer than the tube ID and can result in clogging.
The RFAL fat removal rate is sufficiently better than similarly sized internal diameters of PAL probes. In some embodiments, thermal insulation is added to the RFAL cannula and the OD increases. In some embodiments, a given design shows an advantage in resection rate.
Aperture configurations can vary, but where holes are employed, cross hole configurations allow for easier cleaning should a clog occur.
Dead space at the end of the cannula probably contributes to clogging. Apertures, e.g., holes, should be as distal as possible to avoid such dead space and lesson clogging frequency/likelihood.
End apertures, e.g., end holes, can contribute to reducing dead space and preventing clogging, however end holes present a risk of piercing tissue not present in probes having a rounded tip lacking in aperture holes.
Directionality can be offered by placing apertures (e.g., holes) on only one side of a probe, but this runs counter to the previous observation that distal dead-end flow should be avoided.—Higher removal speeds are possible with radial port designs.
Pulsed energy delivery that has sufficient power to create bubble formation about the apertures results in less clogging as compared to similar powers applied in a CW fashion.
FEP coatings are preferred over PVDF due to their higher melting temperature and robust integrity throughout necessary powers and procedure times.
In some probe embodiments, reducing RF input and staying below 45° C. appears to offer some performance benefits.

### RF Pulsing for Fractionated Coagulation and Generalized Heating

A pulsed RF device can be envisioned with pulse delivery parameters optimized for thermally confined energy delivery with the goal to approach or exceed the performance of the 1444 nm repetitively pulsed laser. The l/e penetration depth of the 1444 nm laser is approximately 1/29.4 cm⁻¹≈340 μm. An RF delivery cannula may be envisioned where the high volumetric energy density deposition volume surrounding the cannula surface will be thinner than 340 μm due to the very fast decrease of RF current density surrounding the outer surface of the cannula. In one embodiment, the high volumetric energy density deposition volume surrounding the cannula surface with range from about 1 μm to about 340 μm, from about 5 μm to about 100 μm, from about 10 μm to about 50 μm.

### Cylindrical Cannula Delivering RF Power Throughout its Surface

A probe having a cylindrically shaped RF delivery cannula positioned near the dermis/fat junction would contact the dermis/fat junction over a narrow stripe or strip on its external cylindrical surface. The rest of its external cylindrical surface will be in contact with adipose tissue infiltrated with saline like tumescent solution. The narrow stripe or strip of the cannula in contact with the dermis is expected to offer lower RF impedance vs the adipose tissue due to the lower dermis impedance. Pulsed RF energy deposition in the dermal stripe can result in thermally confined localized heating and possibly coagulation of all or a portion of the dermal layer in contact with stripe of the cannula in contact with the dermal tissue. When the practitioner moves the cannula to be in contact with another region of the dermis/fat junction and then still another region of the dermis/fat junction, multiple coagulated stripes on the dermis/fat junction can be created over multiple RF pulses delivered under pulsed energy control accommodating variable impedance. Optimization of the width, length and thickness of the coagulated stripes on the dermis/fat junction can be used to optimize the skin tightening effect of the pulsed RF treatment. It is expected that a stripe generated by a given cannula will be approximately the size and/or shape of the portion of cannula in contact with the tissue that it contacts. Due to the higher adipose tissue impedance, a lower volumetric density of RF pulsed energy will be delivered by each pulse in the surrounding tumesced adipose tissue. As a result, from the unconfined heating over multiple on- and off-time cycles little to no localized thermal damage is expected in the adipose tissue while its temperature is rising and providing generalized heating to the overlying dermis. Another advantage of the pulsed RF energy control mode of operation is that it provides consistent average power delivery and generalized heating energy dosing in the tissues near the dermal/fat junction with variable RF impedance. In this way, during liposuction the dermal tissue at the dermal/fat junction may be exposed to heat treatment.

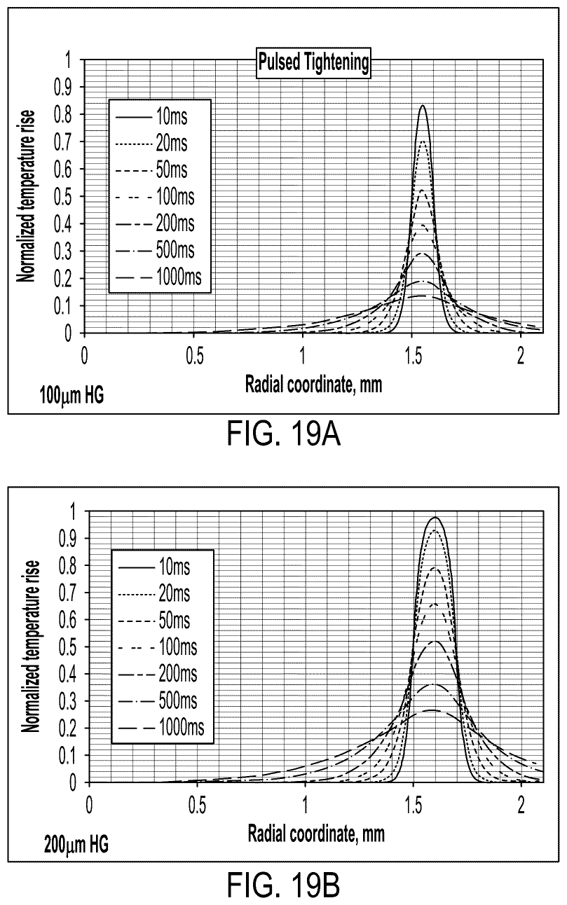

Referring now to FIGS. 19A and 19B and FIG. 5F, a mathematical model for the cylindrical volume of tissue with approximately uniform RF heat generation region surrounding the cylindrical cannula was built with the capability to vary the thickness of the RF heat generation region surrounding the cannula tip and referred to as HG. Consider the probe tip 520 in FIG. 5F as shown to be surrounded by a heat generation region HG that corresponds to the high current density region surrounding the working portion of the cannula 525 or probe tip 520.

Where the cannula 525 of the probe 500 has four apertures 530 (e.g., a first set of through holes in cross configuration with a second set of through holes) as is shown in FIGS. 5F and 5G the insulative coating 550 of PVDF is disposed over the stainless steel cannula and the portions of the PVDF coating 550 that are adjacent to the aperture(s) 530 leave at least a portion of the thickness of the stainless steel cannula surrounding the aperture void is exposed as a conductive material 540 such as stainless steel. In some embodiments, when RF energy is applied to the cannula, the heat leakage through the PVDF coating 550 heats the surrounding tissue (in the absence of suction).

In the model shown in FIG. 19A the thickness of the high current density region HG measures 100 μm and in FIG. 19B the thickness of high current density region HG measures 200 μm. With regard to FIG. 19A, the heat generation region is modeled with thickness of 100 μm and with RF pulse on-times between 10 ms and 1000 ms. With regard to FIG. 19B, the heat generation region is modeled with thickness of 200 μm and with RF pulse on-times between 10 ms and 1000 ms

To simplify the models in FIGS. 19A and 19B, the region HG that surrounds the outer cannula surface with RF heat generation is modeled as being surrounded by tissue with negligible RF heat generation. Accordingly, in some embodiments, the surrounding tissue is modeled as a cylindrical volume of unheated tumesced tissue. The inside volume of the cannula 525 was assumed to be filled saline like aqueous media with thermal diffusivity approximately equal to dermal tissue and without heat generation. The apertures 530 shown in FIG. 5F are modeled to not affect the RF high current density region. The calculated temperature rise at the end of a heating RF pulse in the heat generation region was normalized so that the temperature rise produced by a short pulse with complete heat confinement would be equal to 1. FIGS. 19A and 19A show examples of the calculated normalized temperature rise at the end of a heating RF pulse in an exemplary 3 mm diameter cannula (e.g., a probe cannula similar to that disclosed in relation to FIGS. 5F-5I) and the surrounding tissue for heat generation region HG thicknesses of 100 μm and 200 μm and RF pulse on-times between 10 and 1000 ms. In all considered cases the uniform energy density deposited by the RF pulse is the same.

The plots of FIGS. 19A and 19A demonstrate the benefits of heat confined pulsed RF delivery, specifically, pulsed tightening. Short pulses with on-times 10 to 20 ms deliver the highest temperature rises that are localized in the heat generation region of tissue surrounding the cannula. Longer pulses deliver progressively lower peak temperatures in enlarged surrounding regions. The thickness of the highest temperature rise region needs to be sufficient to lead to clinically significant fractional thermal coagulation of the dermis in the tissue stripe in contact with the cannula. The overall skin response to the multiple striped coagulation injuries and the general dermal heating is expected to lead to a healing response that may include the desired skin tightening effect. In addition, the thickness of the individual coagulated stripe injury and the tissue volume fraction of the multiple stripes should be sufficiently small not to interfere in the normal skin healing process. The thickness and depth of each coagulated stripe injury will be determined in part by the dimension of the cannula of the probe tip with a cannula having a relatively larger area of active RF energy providing a relatively thicker coagulated stripe injury versus the same diameter cannula with a smaller area of active RF energy and/or a smaller amount of active RF energy. Similarly, a relatively smaller diameter cannula with likely have a smaller active RF energy area and thus impart a relatively thinner coagulated stripe injury versus a relatively larger diameter cannula.

Comparison of FIGS. 19A and 19B shows that the thicker 200 μm heat generation region (designated as HG) provides better heat confinement vs the thinner 100 μm region for the considered RF pulse on-times, leading to higher peak temperature rise for identical uniform heat density deposition. Thus, a thicker coagulated stripe of injury should be expected from the heat generation model of FIG. 19B compared to the expected thinner coagulated strip of injury from the heat generation model of FIG. 19A.

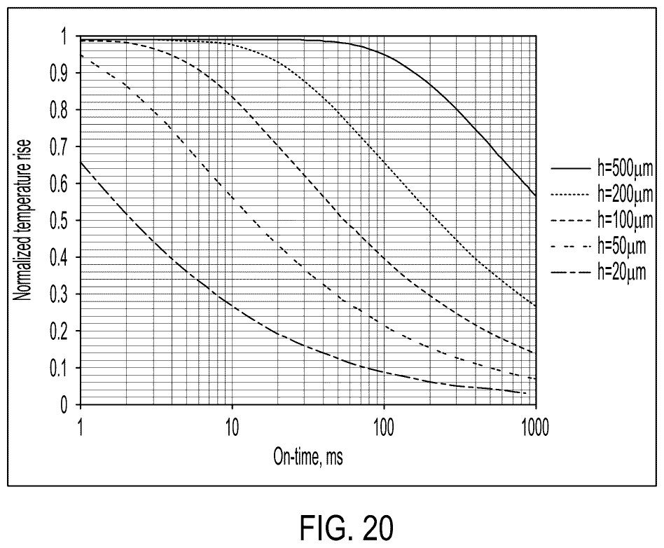

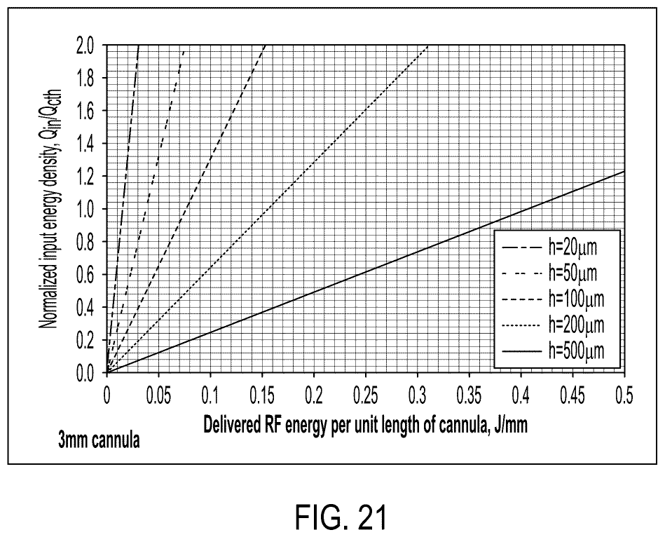

Referring now to FIGS. 20 and 21, the legend provides a range of thickness of high current density region labeled h that measures 20, 50, 100, 200, and 500 μm. The dimension labeled h in FIGS. 20 and 21 is the same as the dimension HG in FIGS. 19A and 19B. In FIG. 20, the calculated peak normalized temperature rises in the RF heated region at the end of the pulse for a range of heat generation region thickness are plotted. The plots are calculated for fixed deposited uniform energy density in the heat generation regions.

The plots on FIG. 20 illustrate the advantages of short pulse on-times especially for smaller thickness of the heat generation region, provided the heating source has sufficient peak power to deliver the required energy density in a short on-time pulse. For example, the plots indicate that for a desired heated and coagulated region thickness between about 100 μm and about 200 μm the preferred pulse duration range should be below about 10 ms to about 20 ms. With regard to FIG. 20, the plots are calculated for fixed deposited uniform energy density in the heat generation regions.

The finite amount of RF power that can be delivered over the time intervals corresponding to heat confinement in the range of considered thicknesses of heat generation region sets a practical limit for the cannula length and diameter. The deposited input energy density can be calculated as

$${Q}_{in} = \frac{{E}_{in}}{1 / 4 π [ {( D + h )}^{2} - {D}^{2} ] L} ,$$

where D and L are the cannula diameter and length, Eᵢₙ is the delivered RF electrical energy and the denominator accounts for the considered volume of the heat generation region.

The threshold temperature for thermal coagulation in skin exposed to a 10 to 20 ms heating pulse is calculated to be about 70° C. See, Welch, A. J., and Martin J C van Gemert. In Optical-Thermal Response of Laser-Irradiated Tissue, Boston, MA: Springer US, 1995. That temperature corresponds to a temperature rise of about ΔT=35° C. from normal skin temperature. The corresponding threshold value for the deposited energy density that leads to thermal coagulation in 10 to 20 ms heating pulse can be calculated as Q_{cth}=ρcΔT=0.16 J/mm³, where ρ=1.2 g/cm³ is the skin density and c=3.8 J/g° K is the skin heat capacity.

Instead of the deposited input energy density, Qᵢₙ, it is more convenient to consider the normalized input energy density, Qᵢₙ/Q_{cth}, values of normalized input energy density above 1 correspond to a necessary condition for forming coagulated stripes on the dermis/fat junction in contact with the RF delivery cannula. The normalized input energy density, Qᵢₙ/Q_{cth}, for a range of delivered RF energy per unit cannula length, Eᵢₙ/L, is plotted on FIG. 21 for a range of thicknesses of the RF heat generation region, h.

The plot in FIG. 21 can be used to estimate the required RF energy per pulse for matching and exceeding the threshold energy density for coagulation. The calculated input energies for 100 and 200 μm thick heat generation regions using a cannula length L=12 mm are approximately Eᵢₙ≈1 J for 100 μm region and Eᵢₙ≈2 J for 200 μm region. The approximate RF pulse powers that will deliver these energies in approximately 10 ms in energy control mode are between 100 and 200 W.

Similar estimates can be made for a 1 mm diameter cannula without suction capabilities that can be used for neck tightening in applications similar to the 1440 nm laser. For a 1 mm cannula, the approximate RF pulse powers that will deliver coagulation threshold energies in approximately 10 ms in energy control mode are estimated between 30 W and 70 W.

### Cylindrical Cannula Delivering RF Power Through Annular Electrodes

Referring now to FIGS. 16A-16B and FIGS. 22 and 23, an alternative design RF delivery cannula can be envisioned with cylindrically shaped electrical insulator coated surface and RF energy delivery predominantly through one or more annular electrodes (e.g., apertures 530) formed by drilling one or more holes though its surface. In a simplified analysis, for an annular shaped RF electrode (e.g., aperture 530 holes) exposed to the aqueous media (e.g., tumesced adipose tissue), the volume of water that will have efficient heat generation can be expected to have a toroidal like shape, or doughnut like shape that compliments the structure of the aperture 530 in the shape of a hole or circle. In an embodiment where the aperture 530 is in the shape of a slot an oblong toroidal shape will be formed. Thus, the heat generation created by the RF energized aperture or electrode will complement the shape of the edges of the aperture where heat generation occurs.

The annular shaped RF electrode (e.g., the aperture(s) 530 having the shape of a hole or circle) when the aperture(s) 530 are energized via pulsed RF energy the most efficient heat generation occurs in the adjacent aqueous media near the edge of the annular shaped RF electrode also referred to as the aperture 530. This heat generation region has a diameter 545 determined by the gradient of the current density which will vary in accordance with the thickness of the conductive region of the aperture, see, e.g., FIGS. 16A and 16B where FIG. 16A shows a diameter 545 that has a relatively larger thickness than the diameter 545 shown in FIG. 16B. Thus, the edge of the annular shaped RF electrode is expected to be in contact with the external surface of the toroid. When the RF delivery cannula is positioned near the dermis/fat junction, the toroidal like heat generation volume of aqueous media generated within the apertures may come sufficiently close to the dermis/fat junction and produce through heat transfer toroidal like volumes of thermally confined localized heating and possibly coagulation of tissue in the dermis and/or the dermis/fat junction. Referring now to FIG. 6D, the heat generation diameter 545 created by the apertures 530 produce coagulated volumes 645 with each coagulated volume 645 having a coagulation region diameter 655.

When the practitioner moves the cannula near the dermis/fat junction, multiple coagulated toroidal like volumes 645 on the dermis/fat junction can be created over multiple RF pulses delivered under pulsed energy control accommodating variable impedance. Due to the relatively higher impedance in adipose tissue, a lower volumetric density of RF pulsed energy will be delivered by each pulse through the annular electrodes exposed to tumesced adipose tissue. In addition, there could be some RF power leakage by capacitive coupling through the electrical insulator coated cannula surface. The unconfined heating over multiple on- and off-time cycles is expected to lead to temperature rise in the adipose tissue with minimal to no localized thermal damage. The temperature rise in the adipose tissue will provide generalized heating to the overlying dermis. Another advantage of the pulsed RF energy control mode of operation is that it provides consistent average power delivery and generalized heating energy dosing in the tissues near the dermal/fat junction with variable RF impedance.

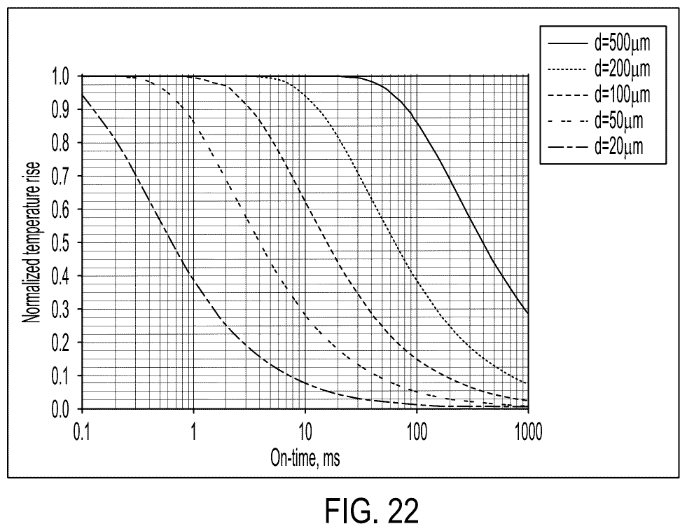

In the present analysis the toroidal-like shape of the volume of water that is heated by each annular electrode will be approximated with a cylinder with variable diameter 545 and length that matches the circumference of the annular shaped RF electrode. The diameter of the cylinder can be chosen so that its volume matches approximately the heat generation water volume in the vicinity of the annular shaped electrode. In the approximate model the cylinders are considered to be heated by a source depositing uniform energy density in their volume. The calculated peak normalized temperature rises in the heated toroidal like regions near the annular RF electrodes at the end of the pulse for a range of heat generation region diameters, d, (e.g., the diameters of the toroidal are plotted on FIG. 22 and are depicted as heat generation region diameter 545 in FIGS. 16A, 16B, and 6D. The plots are calculated for fixed deposited uniform energy density in the heat generation regions.

The plots on FIG. 22 illustrate the advantages of short pulse on-times especially for smaller diameter of the heat generation region, provided the heating source has sufficient peak power to deliver the required energy density in a short on-time pulse. For example, the plots indicate that for desired heat confinement in a heat generation region diameter between 100 and 200 μm the preferred pulse duration range is below 3 to 15 ms. With regard to FIG. 22, the plots are calculated for fixed deposited uniform energy density in the heat generation regions.

The finite amount of RF power that can be delivered over the time intervals corresponding to heat confinement in the range of considered diameters of heat generation region sets a practical limit for the number of annular electrodes on the cannula and their diameter. Assuming heat confinement, the deposited input energy density can be calculated as

$${Q}_{in} = \frac{{E}_{in}}{1 / 4 π {d}^{2}} \text{?} ,$$

$$\text{?} \text{indicates text missing or illegible when filed}$$

where Eᵢₙ is the delivered RF electrical energy and the denominator accounts for the considered cylindrical volume of heated water. In designs where multiple annular electrodes or apertures are desired, S is the total circumferential length of the annular electrode aperture(s).

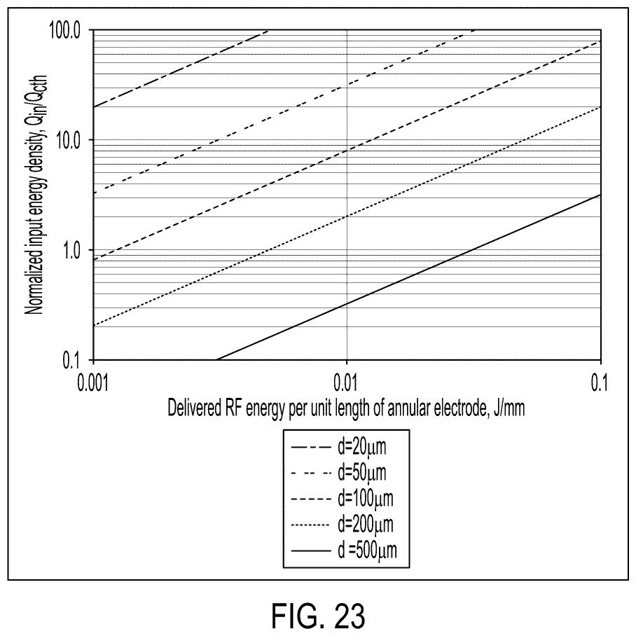

In the preceding section the threshold value for the deposited energy density that leads to thermal coagulation by a heating pulse delivered under the conditions for heat confinement was calculated as Q_{cth}=ρcΔT=0.16 J/mm³. The normalized input energy density, Qᵢₙ/Q_{cth}, for a range of delivered RF energy per unit length of annular electrode(s), Eᵢₙ/S, is plotted on FIG. 23 for a range of diameters of the RF heat generation cylindrical volumes of water, d.

The plot in FIG. 23 can be used to estimate the required RF energy per pulse for exceeding the threshold energy density for coagulation. For input energy per unit length of electrode 0.01 J/mm both 100 and 200 μm diameter heat generation regions d and the diameter heat generation regions 545 are also shown in FIGS. 16A, 16B, and 6D and will be heated above the coagulation threshold. For example, a cannula design with four annular electrodes or stated differently four apertures (see, e.g., 5F-5I), with 3 mm diameter each, will have a total electrode circumference length of 4*π*3 mm≈38 mm, corresponding to RF energy of 0.38 to 0.76 J per pulse for operation 2 to 4 times above the coagulation threshold. Under assumption for heat confinement, the approximate RF pulse powers that will deliver these energies in approximately 3 ms to 15 ms in energy control mode are between 25 and 250 W.

It is remarkable that the calculated RF pulse energy per pulse of 0.38 to 0.76 J/pulse for localized coagulation with a typical cannula with four annular electrodes is substantially smaller than the calculated energy per pulse of 1 to 2 J/pulse for a typical cannula delivering RF power throughout its surface. The four annular electrodes are expected to deliver more localized tissue coagulation and a substantially slower generalized heating and temperature rise in the skin overlaying the treated region provided the two styles of cannulas are used at the same repetition rate of pulsed energy delivery. One possible way of increasing the rate of generalized heating with the four annular electrodes cannula is to increase the repetition rate of pulsed energy delivery to 40 or 50 Hz. These higher repetition rates might be feasible for desired on-times between 3 and 10 ms with the four annular electrodes cannula.

### Further Continuous Mode Embodiments

In one embodiment, a cannula has nine apertures total, specifically, the cannula has three rows containing apertures and each row has three apertures. Each of the set of three apertures in the three rows is separated by 120 degrees from the adjacent aperture in the next row about the circumference of the cannula. In one embodiment, continuous RF power is applied to the cannula with an inner diameter of 2.1 mm and having nine apertures and the cannula was employed to remove tumesced adipose tissue from a patient. The nine apertures pulled in too much tumesced adipose tissue to cause clogging in the cannula. The practitioner attempted to remove the clog by reverse suction and/or manual removal (e.g., via toothbrush or via pick). However, the clog became charred and stuck within the cannula and could not be recovered from such that the cannula could no longer be used for liposuction and needed to be replaced.

### Methods and Devices to Avoid Unwanted Heating of Probe Insertion Region

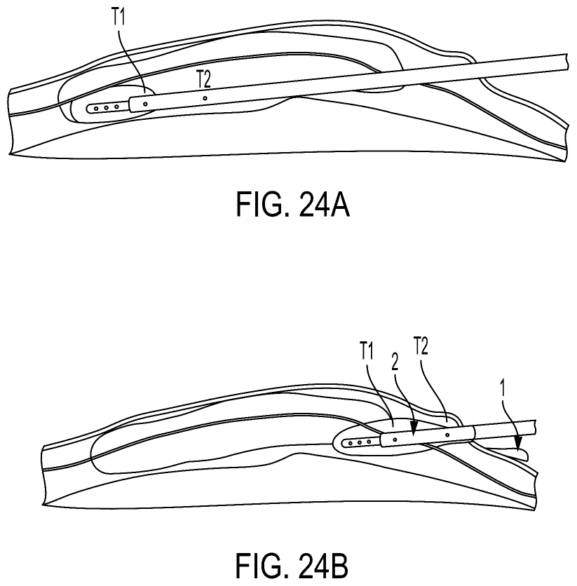

The FIGS. 24A (top) and 24B (bottom) illustrate an exemplary RF dual liposuction and heating probe, as disclosed herein, inserted into a desired patient tissue plane after passing through an incision point. The fat tissue lies beneath the skin bounded beneath by the muscle layer. The temperature at the tip is elevated from the body temp as a result of application of RF energy. Ideally, the heating is confined to that probe tip area, but practically, tissue previously heated via RF energy application becomes a heated aspirate that flows about the outside of the cannula to the incision site during the retraction stroke of the probe illustrated in FIG. 24B. Even with proper insulation at the probe wall, a portion of the heated aspirate from the treatment site may sometimes leak out of the body via the incision site (1). Further, even with proper insulation at the probe wall, the hot probe tip can thermally conduct down the length of the probe thereby heating the external surface of the insulation layer (2).

The leaking of heated aspiration fluid at the treatment site and/or thermal conduction down the external surface of the insulated probe wall can cause unwanted thermal damage to the incision site. In procedures where the heating level of the aspirate and/or probe is relatively high for the area being treated additional measures may be taken to avoid unwanted damage at the incision site. For example, to address and mitigate the risk of unwanted heating at the incision site, a skin port may be employed. The skin port can be a simple plastic insulative bushing that protects the skin incision from friction of probe oscillations and/or thermal events that may cause a burn, effect healing of the incision site, or cause unwanted heating of the incision site. A commercially available skin port can be used to protect the incision site.

Alternatively, one or more skin ports may be employed that are tailored to the RF liposuction probe disclosed herein. For example, in one embodiment, referring to FIGS. 25A-25H, the skin port is thermally insulated such that where the thermally insulated skin port is employed, thermal insulation of the probe is no longer necessary or is less necessary, thus reducing the complexity of the probe and/or reducing complexity of the approach to probe insulation. Several skin port ideas are disclosed here and are depicted in FIGS. 25A-25H.

While commercially available skin ports may penetrate the skin by approximately 1 cm, extensions of the portion that penetrates the skin may offer additional protection. This is significant since the heat buildup of the surrounding tissue may be greater near the fulcrum point of insertion; a deeper skin port could better protect the deeper tissue about the insertion region from unintended heating. A simple solution to this is shown in FIG. 25B where the portion of the port that inserts into the tissue is extended, in this case by 1 cm longer than FIG. 25A.

Suitable skin ports have a tubular protrusion that enters into the skin and is buried to a depth of greater than 5 mm and less than 50 mm. In embodiments disclosed herein, the protrusion buried to a depth in the body of the subject offers thermal protection to the skin by serving as a barrier between an RF probe that is hot and the surrounding skin. The thermal insulation of the skin port tubular protrusion can be achieved through bulk plastic, aerated plastic, tube-in-tube stainless steel configurations and vacuum sealed stainless-steel cannulas. The thermal insulation can be either on the probe or the introducer described here. A commercially available skin port is shown in, for example, FIGS. 25E and 25G.

The introducer design is configured to allow for oscillatory movement of the probe within the tubular introducer as well as freedom to change the angle of approach relative to the skin's surface. Generally, the skin port is anchored to the skin's surface with sutures, staples or may simply be held by the elasticity of the skin. The rings that are shown in FIGS. 25F and 25G-25H represent a way to fix the introducer to the skin with sufficient surface area to overcome the forces imposed when the probe angle shifts inside the subject's body during a treatment procedure.

One method of allowing the angular freedom relative to the skin's surface is shown in FIG. 25D using a ball and socket mechanical arrangement. In this manifestation, the ball and tubular portion of the introducer are integrated and move within a socket formed at the skin surface by two plates that are affixed to the skin. The freedom of the ball and socket can be achieved by a flexible membrane (silicone rubber) that spans between the ring affixed to the skin and the tubular portion of the introducer.

Alternatively, the flexible membrane may be fashioned from a harder plastic but made thin and possibly having cutouts to allow more flexibility. An exemplary design is shown in FIGS. 25F-25H.

Referring now to FIGS. 25G-25H, the following example utilizes a vacuum sealed stainless steel introducer for maximum thermal insulation. This is designed as two pieces: one ring-like piece with the flexible membrane described above and a locking receptacle for a second introducer piece can be sutured or stapled to the skin and the second introducer piece can be locked to the first piece or unlocked to decouple from the surgical incision site. If the protruding portion of the cannula restricts the angular swing of the cannula in tissue, one can unlock the tubular part, pull it out, reinsert it at a new angle and lock it in for a new approach.

Systems and methods utilizing energy to cut, remove, and/or treat adipose tissue and other tissue using an integrated system and one or more probes are described herein. In various aspects, the present teachings can provide a cooled (or uncooled) treatment to achieve one or more of body sculpting (lipolysis), skin tightening (laxity improvement), cellulite treatment apparatus, tissue rejuvenation, and treatment of other conditions, by way of non-limiting examples. In some embodiments, RF-based probes may be used that operate in a pulsed mode or a continuous mode. In many embodiments, one or more probes and the system are configured for radio frequency assisted liposuction and/or RF-based probes that generate bubbles that enhance treatment or removal upon the collapse of such bubbles.

It will be appreciated that for clarity, this disclosure explicates various aspects of embodiments of the applicant's teachings, while omitting certain specific details wherever convenient or appropriate to do so. For example, discussion of like or analogous features in alternative embodiments may be somewhat abbreviated. Well-known ideas or concepts may also for brevity not be discussed in any great detail. The skilled person will recognize that some embodiments of the applicant's teachings may not require certain of the specifically described details in every implementation, which are set forth herein only to provide a thorough understanding of the embodiments. Similarly, it will be apparent that the described embodiments may be susceptible to alteration or variation according to common general knowledge without departing from the scope of the disclosure. The following detailed description of embodiments is not to be regarded as limiting the scope of the applicant's teachings in any manner.

The terms “about” and “substantially and “approximately” as used herein, refer to variations in a numerical quantity that can occur, for example, through measuring or handling procedures in the real world; through inadvertent error in these procedures; through differences/faults in the manufacture of electrical elements; through electrical losses; as well as variations that would be recognized by one skilled in the art as being equivalent so long as such variations do not encompass known values practiced by the prior art. Typically, the term “about” means greater or lesser than the value or range of values stated by 1/10 of the stated value, e.g., ±10%. For instance, applying a voltage of about +3V DC to an element can mean a voltage between +2.7V DC and +3.3V DC. Likewise, wherein values are said to be “substantially identical,” the values may differ by up to 5%. Whether or not modified by the term “about” or “substantially” identical, quantitative values recited in the claims include equivalents to the recited values, e.g., variations in the numerical quantity of such values that can occur, but would be recognized to be equivalents by a person skilled in the art.

Moreover, nothing disclosed herein is intended to be dedicated to the public regardless of whether such disclosure is explicitly recited in the claims. To aid the Patent Office and any readers of any patent issued on this application in interpreting the claims appended hereto or otherwise presented throughout prosecution of this or any continuing patent application, applicants wish to note that they do not intend any claimed feature to be construed under or otherwise to invoke the provisions of 35 USC 112 (f), unless the phrase “means for” or “step for” is explicitly used in the particular claim.

All of the drawings submitted herewith include one or more ornamental features and views, each of which include solid lines any of which also incorporate and correspond to and provide support for dotted lines and alternatively, each of which include dotted lines any of which also incorporate and correspond to and provide support for solid lines.

The use of the terms “include,” “includes,” “including,” “have,” “has,” or “having” should be generally understood as open-ended and non-limiting unless specifically stated otherwise.

The use of the singular herein includes the plural (and vice versa) unless specifically stated otherwise. Moreover, the singular forms “a,” “an,” and “the” include plural forms unless the context clearly dictates otherwise. In addition, where the use of the term “about” is before a quantitative value, the present teachings also include the specific quantitative value itself, unless specifically stated otherwise.

It should be understood that the order of steps or order for performing certain actions is immaterial so long as the present teachings remain operable. Moreover, two or more steps or actions may be conducted simultaneously.

Where a range or list of values is provided, each intervening value between the upper and lower limits of that range or list of values is individually contemplated and is encompassed within the disclosure as if each value were specifically enumerated herein. In addition, smaller ranges between and including the upper and lower limits of a given range are contemplated and encompassed within the disclosure. The listing of exemplary values or ranges is not a disclaimer of other values or ranges between and including the upper and lower limits of a given range.

It should be appreciated that numerous changes can be made to the disclosed embodiments without departing from the scope of the present teachings. While the foregoing figures and examples refer to specific elements, this is intended to be by way of example and illustration only and not by way of limitation. It should be appreciated by the person skilled in the art that various changes can be made in form and details to the disclosed embodiments without departing from the scope of the teachings encompassed by the appended claims.

1. A cosmetic tissue treatment probe, comprising:
a cannula having a length, the cannula comprising
a conductive material,
an inner surface,
an outer surface,
a cannula wall disposed between the inner surface and the outer surface,
a proximal end,
a distal end,
a tip,
the cannula wall having a cannula wall thickness and a cannula length,
wherein at least a portion of the tip is defined by the distal end, the proximal end attachable to a handle; and
one or more apertures defined by the cannula wall,
wherein the conductive material is configured for electrical communication with an RF generator, the conductive material configured to generate a heat effect in response to a RF signal received from the RF generator, wherein the heat effect is generated relative to a medium or tissue in fluid communication with the one or more apertures.

2. The probe of claim 1, wherein the RF signal has one or more probe parameters, the one or more probe parameters selected to adjust or achieve the heat effect or a heat effect parameter.

3. The probe of claim 2, wherein the one or more probe parameters of the RF signal that are selected include at least two of pulsed RF, continuous wave RF, peak power of the RF signal, average power of RF signal, a repetition rate of RF signal, on-time of the RF signal and a RF frequency.

4. The probe of claim 2, wherein the heat effect parameter is selected from one or more of the following: volume of heating, duration of heating, gradient of heating, amount of heat localization, heating efficiency for localized change of state or steam formation, heating efficiency for localized tissue effects, and RF efficiency for localized change of state or plasma formation, and heating efficiency for localized change of state or plasma formation.

5. The probe of claim 4, wherein a heating efficiency for localized tissue effects include one or more of more of localized tissue coagulation, localized tissue ablation or vaporization, localized mechanical disruption, localized thermo-mechanical tissue disruption, localized pressure disruption, or localized shockwave tissue disruption.

6. The probe of claim 2, wherein the one or more probe parameters are peak power, average power, an energy per pulse and a repetition rate, wherein the pulsed RF signal has a peak power having a range of about 200 W to about 3 kW, an average power having a range of about 5 W to about 100 W, an energy per pulse range of about 1 J to about 5 J per pulse and a repetition rate range of about 5 Hz to about 100 Hz.

7. The probe of claim 2 wherein the one or more probe parameters are peak power, average power, an energy per pulse, wherein the pulsed RF energy signal has a peak power having a range of about 200 W to about 3 KW, an average power having a range of about 0.1 W to about 10 W, an energy per pulse range of about 1 J to about 5 J per pulse and a repetition rate of about 0.5 Hz to about 10 Hz.

8. The probe of claim 2 wherein the one or more probe parameters are peak power, average power, an energy per pulse, wherein the pulsed RF signal has a peak power having a range of about 200 W to about 3 kW, an average power having a range of about 1 W to about 60 W, an energy per pulse range of about 0.5 J to about 3 J per pulse and a repetition rate of about 1 Hz to about 60 Hz.

9. (canceled)

10. The probe of claim 1, wherein the one or more apertures are slots defined by the inner surface and the exterior surface of the cannula, wherein each slot defines a channel disposed through the cannula wall.

11. The probe of claim 1, wherein the one or more apertures comprises a first aperture having a first perimeter and a second aperture having a second perimeter, wherein a shape of the first perimeter is substantially the same as a shape of the second perimeter, wherein the cannula has a longitudinal axis, wherein a first point within the first perimeter and a second point within the second perimeter are colinear along a line segment substantially perpendicular to the longitudinal axis.

12. (canceled)

13. The probe of claim 2 further comprising an insulative layer disposed on at least a portion of a surface or edge of the cannula.

14. The probe of claim 1 wherein the cannula further comprises a port for communication with a first pump or a second pump, wherein the first pump is a suction pump, the suction pump configured suction pump for removal of unwanted tissue through the cannula.

15. (canceled)

16. The probe of claim 1 wherein application of the RF signal is selected based on one or more parameters of the RF signal to promote heat generation regions in an aqueous medium surrounding the cannula.

17. The probe of claim 1 further comprising a control system, the control system in electrical communication with one or more electrical contacts of the probe, wherein the control system is programmed to terminate delivery of an RF signal during treatment when a measured reactance value is received or generated by the control system that is indicative of plasma formation.

18. The cosmetic tissue treatment probe of claim 1 wherein application of the pulsed RF energy signal initiates, promotes or causes formation of one or more heat bubbles in an aqueous medium surrounding the cannula and in fluid communication with the one or more apertures.

19. (canceled)

20. (canceled)

21. (canceled)

22. (canceled)

23. The cosmetic tissue treatment probe of claim 13 wherein the insulative layer is disposed on the exterior surface of the cannula but leaves conductive material of the cannula wall thickness and/or the exterior surface exposed at the one or more apertures.

24. The cosmetic tissue treatment probe of claim 13 wherein the insulative layer is disposed on the surface of the cannula but leaves conductive material exposed about the one or more apertures on the exterior surface of the cannula.

25. (canceled)

26. The cosmetic tissue treatment probe of claim 13 wherein the insulative layer is heat shrunk about the exterior surface of the cannula.

27. The cosmetic tissue treatment probe of claim 13 wherein the insulative layer is a coating disposed about the exterior surface of the cannula.

28. A cosmetic tissue treatment system comprising:
a housing;
a first pump disposed in the housing;
a RF generator disposed in the housing; the RF generator configured to output treatment energy signals;
a power supply disposed in the housing, the power supply in electrical communication with the first pump and the RF generator; a
a control system disposed in the housing, the control system in electrical communication with the RF generator and/or the first pump, the control system is configured to operate the RF generator in one or more of a pulsed RF energy mode and a continuous RF power mode;
one or more probe connectors; and
a tissue treatment probe having a length, the probe connected to one or more probe connectors, the probe comprising a cannula comprising a conductive material, the cannula having a wall thickness, the cannula comprising a cannula wall, an inner surface, an outer surface, a proximal end, a length, a distal end, and a tip disposed is in a region of the distal end, the proximal end attachable to a handle, one or more apertures defined by the cannula wall, wherein application of an RF energy signal to an electrical contact of the probe generates a heat effect, wherein the heat effect is generated relative to a medium or tissue in fluid communication with the one or more apertures.

29.-31. (canceled)

32. The cosmetic tissue treatment system of claim 1, wherein the one or more probe parameters of the RF energy signal that are selected include at least one of pulsed RF, continuous wave RF, peak power of the RF energy signal, average power of RF energy signal, a repetition rate of RF signal, on-time of the RF signal and a RF frequency.

33.-68. (canceled)

69. A cosmetic tissue treatment method, comprising:
providing a tissue treatment probe comprising a cannula comprising a conductive material, the cannula having a cannula wall thickness, and having a proximal end, a length, and a distal end, a tip is in the region of the distal end, the proximal end is coupled to a handle, one or more apertures are disposed through the wall thickness;
inserting the distal end of the probe into a region of tissue targeted for cosmetic treatment; and
applying an RF energy signal to the cannula to generate a heat effect on a medium or tissue in fluid communication or adjacent the one or more apertures, wherein the RF energy signal that is applied has one or probe more parameters, the one or more probe parameters selected to adjust or achieve the heat effect or a heat effect parameter.

70. The cosmetic tissue treatment method of claim 69, wherein the one or more probe parameters of the RF energy signal that are selected include at least one of pulsed RF, continuous wave RF, peak power of the RF energy signal, average power of RF energy signal, a repetition rate of RF signal, on-time of the RF signal and a RF frequency.

71. The cosmetic tissue treatment method of claim 69, wherein the heat effect parameter is selected from one or more of the following: volume of heating, duration of heating, gradient of heating, amount of heat localization, heating efficiency for localized change of state or steam formation, heating efficiency for localized tissue effects, RF efficiency for localized change of state or plasma formation, and heating efficiency for localized change of state or plasma formation.

72. The cosmetic tissue treatment method of claim 71, wherein a heating efficiency for localized tissue effects include one or more of more of localized tissue coagulation, localized tissue ablation or vaporization, localized mechanical disruption, localized thermo-mechanical tissue disruption, localized pressure disruption, or localized shockwave tissue disruption.

73. (canceled)

74. (canceled)

75. The cosmetic tissue treatment method of claim 73, further comprising a first pump, wherein the first pump is a suction pump for removal of at least a portion of an aqueous medium comprising tumescence and unwanted adipose tissue from the region of tissue targeted for cosmetic treatment through the cannula.

76. (canceled)

77. The cosmetic tissue treatment method of claim 69, wherein application of the pulsed RF energy signal causes heat confinement in the aqueous medium surrounding the cannula to cause rapid vaporization of a portion of the aqueous medium present in the region of the aperture thereby forming one or more bubbles.

78. The cosmetic tissue treatment method of claim 77, wherein forming the bubbles is followed by collapse of one or more of the formed bubbles thereby contributing to breakdown of the adipose tissue in the aqueous medium.

79. (canceled)

80. The cosmetic tissue treatment method of claim 7779, wherein the pulsed RF energy signal applies pulsed RF energy that ranges from about 1 Joule per pulse to about 5 Joules per pulse and delivered at a pulse frequency that ranges from about 5 Hz to about 100 Hz.

81. The cosmetic tissue treatment method of claim 77, wherein the cannula is connected to a first pump and the first pump is a suction pump for removal of at least a portion of the aqueous medium from the region of tissue targeted for cosmetic treatment through the cannula, wherein the bubbles disrupt all or a portion of any blockage in the one or more apertures thereby reducing or avoiding interruption of aqueous medium removal through the cannula.

82. The cosmetic tissue treatment method of claim 81, wherein the pulsed RF energy signal applies pulsed RF energy that ranges from about 1 Joule per pulse to about 5 Joules per pulse and delivered at a pulse frequency that ranges from about 0.5 Hz to about 10 Hz.

83. The method of claim 69 further comprising measuring impedance on a real-time basis relative to a localized treatment region; and automatically adjust applied voltage of RF energy signal, using a control system, in response to a change in an impedance value measured with regard to the localized treatment region, wherein the applied voltage of the RF energy signal is adjusted in response to the change in impedance value such that the RF energy signal is delivered with a maximum peak power level.

84. The method of claim 69 further comprising compensating for impedance variability during a cosmetic treatment by setting a pulse duration limit, and discontinuing treatment when the pulse duration limit is met.

85. The method of claim 69 further comprising measuring reactance and terminating the delivery of an RF energy signal during treatment when a reactance value is measured that is indicative of plasma formation.

86. The method of claim 69 further comprising measuring reactance, allowing plasma formation for a time period when a reactance value is measured that is indicative of plasma formation and terminating the delivery of an RF energy signal upon the expiration of the time period.

87. The method of claim 69 further comprising initiating, promoting or causing formation of one or more heat bubbles in an aqueous medium surrounding the cannula and in fluid communication with the one or more apertures in response to application of the RF energy signal.

88. The method of claim 69 further comprising generating bubbles in response to delivery of RF energy to tissue or an aqueous medium in contact with one or more apertures of the cannula; and providing audible or tactile feedback indicative of reaching a target per pulse energy delivery suitable for performing a cosmetic procedure.

89. The method of claim 69, wherein the RF energy signal is pulsed.

90.-99. (canceled)

100. The cosmetic tissue treatment system of claim 29, wherein the RF energy signal has one or more probe parameters, the one or more probe parameters selected to adjust or achieve the heat effect or a heat effect parameter, wherein the one or more probe parameters of the RF energy signal that are selected include at least one of pulsed RF, continuous wave RF, peak power of the RF energy signal, average power of RF energy signal, a repetition rate of RF signal, on-time of the RF signal and a RF frequency.
